(19) United States
(12) Patent Application Publication
(10) Pub. No.: US 2026/0124200 A1
(43) Pub. Date: May 7, 2026

(54) METHOD OF TREATING NEURODEGENERATIVE DISEASES

(71) Applicants: The Board of Trustees of the University of Illinois, Urbana, IL (US); The Regents of the University of Michigan, Ann Arbor, MI (US)

(72) Inventors: Martin D. Burke, Champaign, IL (US); Stella Ekaputri, Urbana, IL (US); Samantha Taylor Brown, Columbus, OH (US); Nathan Eric Schroeder, St. Joseph, IL (US); Young Ah Seo, Ann Arbor, MI (US)

(21) Appl. No.: 19/116,224

(22) PCT Filed: Oct. 2, 2023
(86) PCT No.: PCT/US23/34290

§ 371 Date: Mar. 27, 2025

Related U.S. Application Data
(60) Provisional application No. 63/411,902, filed on Sep. 30, 2022.

(51) Int. Cl. A61K 31/505; A61K 9/00; A61K 31/122; A61K 31/337; A61K 31/341; A61K 31/343; A61K 31/351; A61K 31/381; A61K 31/4196; A61K 31/421; A61K 31/426; A61K 31/4412; A61K 31/4418; A61K 31/4436; A61K 31/4465; A61P 25/28

(52) U.S. Cl. CPC A61K 31/505; A61K 9/0019; A61K 31/122; A61K 31/337; A61K 31/341; A61K 31/343; A61K 31/351; A61K 31/381; A61K 31/4196; A61K 31/421; A61K 31/426; A61K 31/4412; A61K 31/4418; A61K 31/4436; A61K 31/4465; A61P 25/28

(57) ABSTRACT

Disclosed is a method of treating a disease or condition characterized by neurodegeneration via administering to a subject in need thereof a therapeutically effective amount of a compound selected from the group consisting of hinokitiol, hinokitiol derivatives, and iron-transporting tropolones.

### RELATED APPLICATIONS

This application claims the benefit of priority to U.S. Provisional Application No. 63/411,902, filed Sep. 30, 2022.

### STATEMENT OF GOVERNMENT SUPPORT

This invention was made with government support under 1R01HL140526 awarded by the National Institutes of Health. The government has certain rights in the invention.

### BACKGROUND

Iron misdistribution underlies many neurodegenerative diseases. For example, the most prevalent neurodegenerative diseases, such as Alzheimer's disease and Parkinson's disease, are associated with brain iron accumulation that progressively increases with age. Less prevalent diseases, such as Friedreich's ataxia and Huntington's disease, are also characterized by abnormal iron accumulation in the brain. Specifically, it has been reported that Friedreich's ataxia patients accumulate iron inside the mitochondria, which suggests that cellular iron misdistribution also contributes to the pathology. See Michael Li-Hsuan Huang, Darius J. R. Lane, and Des R. Richardson; “Mitochondrial Mayhem: The Mitochondrion as a Modulator of Iron Metabolism and Its Role in Disease”; Antioxidants & Redox Signaling, 2011, 15:12, 3003-3019. Significant correlation between brain iron overload and neurodegeneration is also observed in the disease known as neurodegeneration with brain iron accumulation (NBIA), a group of inherited neurological disorders marked by neurological deterioration in early adulthood along with progressive dystonia, Parkinsonism, cognitive decline, and seizures.

The innate ability of iron to act as both an electron acceptor and an electron donor makes it essential for most living organisms. The iron demand is particularly high in the brain, the most metabolically active organ in the body, as iron is needed for oxidative metabolism, mitochondrial energy generation, synaptic plasticity, myelination, and the synthesis of neurotransmitters. However, excess iron, especially in Fe(II) form, is neurotoxic because of its ability to generate deleterious reactive oxygen species (ROS) via the Fenton reaction of the Haber-Weiss cycle, resulting in oxidative injury that can lead to cell death. Therefore, brain iron overload is a critical yet underappreciated factor that contributes to neurodegenerative diseases.

The effective reversal of brain iron overload will require compounds that enable site-specific transmembrane iron mobilization in a safe manner by maintaining bound iron as Fe(III). Ideally, these molecules would also functionally interface with endogenous iron-binding small molecules and proteins. Currently, iron chelators, such as deferasirox (DFX) and deferiprone (DFP), are promising drug candidates for protection against iron-associated neurodegeneration. However, DFP can cause serious side effects, such as agranulocytosis, arthropathy, gastrointestinal bleeding, ophthalmic/auditory toxicity, the loss of essential nutrients (zinc and copper), and neurological complications. Moreover, DFP and other clinically approved iron chelators operate primarily by removing iron that has already been released from cells; i.e., they cannot directly transport iron across lipid bilayers. Thus, their potential for removing iron from neurons and/or their supporting cells is limited.

Accordingly, there exists a need for a new method of treatment for neurodegenerative diseases that is highly specific, well-tolerated, and can serve as a useful therapy.

### SUMMARY

In certain embodiments, the present disclosure is to a method of treating a disease or condition selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA, comprising administering to a subject in need thereof a therapeutically effective amount of a compound selected from the group consisting of hinokitiol, hinokitiol derivatives, and iron-transporting tropolones.

In certain embodiments, the present disclosure provides a method of treating a disease or condition a disease or condition selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA, comprising administering to a subject in need thereof a therapeutically effective amount of a compound represented by any one of Formulas (I), (Ia), (Ib), (Ic), (Id), (IIa), (IIb), and (IIc):

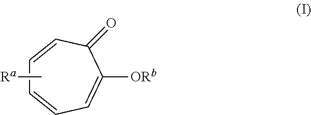

R^{a} is C₁₋₂₀-alkyl, C₂₋₂₀-alkenyl, C₂₋₂₀-alkynyl, C₃₋₉-cycloalkyl, aryl, or heteroaryl, each of which is unsubstituted or substituted with a substituent selected from the group consisting of halo, NO₂, CN, C₁₋₆-alkyl, C₁₋₆-haloalkyl, and C₁₋₆-alkoxy; and
R^{b} is hydrogen or methyl; provided the compound is not hinokitiol;

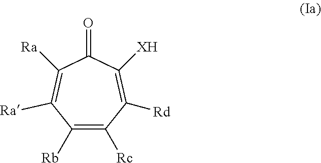

X represents oxygen or sulfur;
Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl; and
Rₐ′ represents hydrogen, halo, alkyl, or substituted alkyl; and R_{b}, R_{c}, and R_{d} are independently selected from the group consisting of hydrogen, halo, alkyl, substituted alkyl, heteroalkyl, alkylcycloalkyl, alkylheterocycloalkyl, alkoxy, substituted alkoxy, alkoxyalkyl, substituted alkoxyalkyl, cycloalkyloxy, substituted cycloalkyloxy, heterocycloalkyloxy, substituted heterocycloalkyloxy, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl; provided that Rₐ, R_{b}, R_{c}, and R_{d} are not all hydrogen;

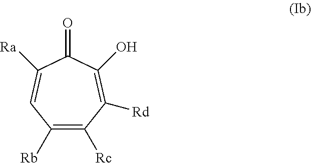

Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl; and
R_{b}, R_{c}, and R_{d} are independently selected from the group consisting of hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl; provided that Rₐ, R_{b}, R_{c}, and R_{d} are not all hydrogen;

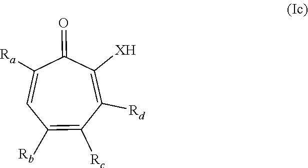

X represents sulfur or oxygen;
Rₐ, R_{b}, R_{c}, and R_{d} independently represent hydrogen, halo, alkyl, substituted alkyl, heteroalkyl, alkoxy, substituted alkoxy, alkoxyalkyl, substituted alkoxyalkyl, aryloxy, substituted aryloxy, heteroaryloxy, substituted heteroaryloxy, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, heteroalkynyl, aryl, substituted aryl, heteroaryl, or substituted heteroaryl; and
   at least one of Rₐ, R_{b}, R_{c}, and R_{d} is aryloxy, substituted aryloxy, heteroaryloxy, substituted heteroaryloxy, aryl, substituted aryl, heteroaryl, substituted heteroaryl; provided that Rₐ, R_{b}, R_{c}, and R_{d} are not all hydrogen;

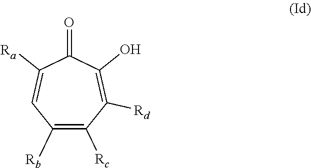

Rₐ, R_{b}, R_{c}, and R_{d} independently represent hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, heteroalkynyl, aryl, substituted aryl, heteroaryl, substituted heteroaryl;
   at least one of R_{b}, R_{c}, and R_{d} is aryl, substituted aryl, heteroaryl, or substituted heteroaryl; and provided that Rₐ, R_{b}, R_{c}, and R_{d} are not all hydrogen; and

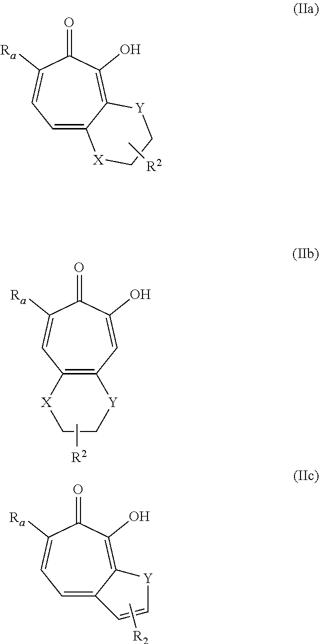

Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
   X and Y independently represent O, S, NH, or CR₅R₆;
   R₂ represents —F, alkyl, haloalkyl, or alkoxy; and
   R₅ and R₆ represent independently for each occurrence H, (C1-C15) alkyl, or substituted (C1-C15)alkyl;
   provided the compound is not

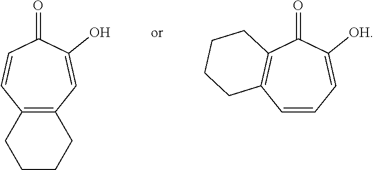

In certain embodiments, the compounds used in treating a disease or condition characterized by neurodegeneration include pharmaceutically acceptable salts, tautomers, and isomers. The compounds can be administered as a single compound or a combination of the compounds or in combination with one or more chelators (examples include deferasirox (DFX) and deferiprone (DFP)). The compounds may be combined with a pharmaceutically acceptable carrier or excipient administered systemically, orally or intravenously to a mammal.

In certain embodiments the disease or condition is selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA.

In certain embodiments the disease or condition is selected from the group consisting of a vascular dementia, a tauopathies, a progressive supranuclear palsy, a corticobasal degeneration, a subacute sclerosing panencephalitic parkinsonism, a postencephalitic parkinsonism, a guam parkinsonism-dementia complex, a Pick's disease, and a frontotemporal dementia.

### BRIEF DESCRIPTION OF THE DRAWINGS

FIG. 1A depicts the reactivity of various Fe(III)-compound complexes to be reduced to Fe(II) measured using Ferrozine assay in reducing conditions.

FIG. 1B depicts the generation of ROS in a mixture of Fe(II) and various compounds measured using hydroxy radical probe 2,7-dichlorofluorescin (DCF) dye.

FIG. 1C depicts EPR measurement of Fe(II) alone.

FIG. 1D depicts EPR measurement of Fe(II) and hinokitiol.

FIG. 2A depicts the structure of FeM-1269.

FIG. 2B depicts particle size measurement using DLS as an indicator of Fe-compound aggregation.

FIG. 2C depicts measurement of iron efflux from liposomes.

FIG. 2D depicts measurement of iron efflux from FPN1-KD Caco-2 cells using hinokitiol and FeM-1269.

FIG. 3A-FIG. 3H depict treatment of fpn-1.2KO C. elegans with various compounds for neurodegeneration scoring and iron level measurement.

FIG. 3A depicts treatment scheme of fpn-1.2KO; Pdat::GFP worms treated with various concentrations of DFP, Hino, and AMB-1269. Dopaminergic neurodegeneration was scored blinded by phenotypic analyses.

FIG. 3B depicts dopaminergic neurodegeneration scoring of fpn-1.2KO worms treated with DFP.

FIG. 3C depicts dopaminergic neurodegeneration scoring of fpn-1.2KO worms treated with Hino.

FIG. 3D depicts dopaminergic neurodegeneration scoring of fpn-1.2KO worms treated with FeM-1269.

FIG. 3E depicts treatment scheme of fpn-1.2KO; Pftn-1::GFP worms treated with various concentrations of DFP, Hino, and FeM-1269.

FIG. 3F depicts ASI neurons (below) which expresses GFP-tagged ferritin levels, Fluorescence levels of fpn-1.2KO worms treated with DFP. * P<0.05, ** P<0.01. ***

FIG. 3G depicts ASI neurons (below) which expresses GFP-tagged ferritin levels, Fluorescence levels of fpn-1.2KO worms treated with Hino. * P<0.05, ** P<0.01, ***

FIG. 3H depicts ASI neurons (below) which expresses GFP-tagged ferritin levels, Fluorescence levels of fpn-1.2KO worms treated with FeM-1269. * P<0.05, ** P<0.01. ***

FIG. 4A-FIG. 4E depicts flatiron mice which display increased anxiety and reduced exploratory activity. WT and flatiron mice were subjected to elevated plus maze task for assessment of anxiety-like behavior.

FIG. 4A depicts total distance traveled in whole maze and average velocity.

FIG. 4B depicts time spent in open arms and center zone.

FIG. 4C depicts rearing frequency and duration. * P<0.05 by Student's t-test.

FIG. 4D depicts ICP-MS measurement of brain iron level in flatiron mice after acute treatment of Hino through intraperitoneal (IP) injection.

FIG. 4E depicts ICP-MS measurement of brain iron level in flatiron mice after chronic treatment of Hino through intraperitoneal (IP) injection. ** P<0.01, *** P<0.001 by one-way ANOVA.

FIG. 5A depicts a schematic of a wild type cellular ceruloplasmin phenotype.

FIG. 5B depicts a schematic of a cellular aceruloplasminemia phenotype.

FIG. 5C depicts a schematic of hinokitiol oxidizing iron and restoring iron homeostasis to a cell of an aceruloplasminemia phenotype.

FIG. 6A depicts a cyclic voltammogram of Fe(Hino)₃.

FIG. 6B depicts cyclic voltammograms of various Fe:Hino ratios.

FIG. 7A depicts EPR measurements of hinokitiol-promoted oxidation of Fe(II).

FIG. 7B depicts EPR measurements of FeM-1269-promoted oxidation of Fe(II).

FIG. 8A depicts a schematic of iron handoff between Fe(Hino)₃ and transferrin.

FIG. 8B depicts a western blot of transferrin in the presence of increasing concentrations of Fe(Hino)₃.

FIG. 8C depicts kinetics of iron handoff between Fe(Hino)₃ and transferrin.

FIG. 9 depicts a colorimetric assay determining the percent conversion from Fe(III) to Fe(II) in the presence in the presence of a strong reducing agent.

FIG. 10 depicts a fluorometric assay determining the quantity of reactive oxygen species (ROS) produced in the Fe(II)-catalyzed Fenton reaction.

FIG. 11A depicts a measurement of dopamine oxidation in vitro in the presence of hinokitiol and Fe(II) or Deferiprone (DFP) and Fe(II).

FIG. 11B depicts a measurement of dopamine oxidation in day five fpn1.2KO C. elegans post treatment of hinokitiol or deferiprone (DFP).

FIG. 12A depicts the quantity of dopamine remaining following Fe(II)-catalyzed oxidation in the presence of hinokitiol and FeM-1269.

FIG. 12B depicts the quantity of dopaminochrome (DAC) produced following Fe(II)-catalyzed oxidation in the presence of hinokitiol and FeM-1269.

FIG. 13 depicts an absorbance vs. concentration plot for DAC vs. peak area following quenching with glutathione.

FIG. 14 depicts the percent conversion to Fe(II) vs. time for Fe(III) precomplexed with hinokitiol or dopamine.

### DETAILED DESCRIPTION OF THE INVENTION

### Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or claims, such terms are intended to be inclusive in a manner similar to the term “comprising”.

The term “about” or “approximately” as used herein means within an acceptable error range for the particular value as determined by one of ordinary skill in the art, which will depend in part on how the value is measured or determined, i.e. the limitations or the measurement system. For example, “about” can mean a range of up to 20%, up to 15%, up to 10%, up to 5%, or up to 1% of a given value.

The term “acyl” is a term as used herein refers to any group or radical of the form RCO— where R is any organic group, e.g., alkyl, aryl, heteroaryl, aralkyl, and heteroaralkyl. Representative acyl groups include acetyl, benzoyl, and malonyl.

The term administration (administering) as used herein refers to the compounds may be provided orally, by intralesional, intraperitoneal, intramuscular or intravenous injection; infusion; liposome-mediated delivery; topical, nasal, anal, vaginal, sublingual, urethral, transdermal, intrathecal, ocular or otic delivery. In order to obtain consistency in providing the compound of this invention it is preferred that a compound of the invention is in the form of a unit dose. Suitable unit dose forms include tablets, capsules and powders in sachets or vials. Such unit dose forms may contain from 0.1 to 300 mg of a compound of the invention and preferably from 2 to 100 mg. Still further preferred unit dosage forms contain 5 to 50 mg of a compound of the present invention. The compounds of the present invention can be administered orally at a dose range of about 0.01 to 100 mg/kg or preferably at a dose range of 0.1 to 10 mg/kg. Such compounds may be administered from 1 to 6 times a day, more usually from 1 to 4 times a day. The effective amount will be known to one of skill in the art; it will also be dependent upon the form of the compound. One of skill in the art could routinely perform empirical activity tests to determine the bioactivity of the compound in bioassays and thus determine what dosage to administer. The compound of may be delivered locally via a capsule that allows a sustained release of the compound over a period of time. Controlled or sustained release compositions include formulation in lipophilic depots (for example: fatty acids, waxes, oils).

The term “alkenyl” or “alkenyl group” means a group formed by removing a hydrogen from a carbon of an alkene, where an alkene is an acyclic or cyclic compound consisting entirely of hydrogen atoms and carbon atoms, and including at least one carbon-carbon double bond. An alkenyl group may include one or more substituent groups.

The term “alkoxy” or “alkoxy group” as used herein means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy. The terms “alkyenyloxy”, “alkynyloxy”, “carbocyclyloxy”, and “heterocyclyloxy” are likewise defined.

The term “alkyl” as used herein is a term of art and refers to saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight-chain or branched-chain alkyl has about 30 or fewer carbon atoms in its backbone (e.g., C1-C30 for straight chain, C3-C30 for branched chain), and alternatively, about 20 or fewer. In one embodiment, the term “alkyl” refers to a C1-C10 straight-chain alkyl group. In one embodiment, the term “alkyl” refers to a C1-C6 straight-chain alkyl group. In one embodiment, the term “alkyl” refers to a C3-C12 branched-chain alkyl group. In one embodiment, the term “alkyl” refers to a C3-C8, branched-chain alkyl group. Cycloalkyls have from about 3 to about 10 carbon atoms in their ring structure, and alternatively about 5, 6, or 7 carbons in the ring structure.

The term “alkylene” is art-recognized, and as used herein pertains to a diradical obtained by removing two hydrogen atoms of an alkyl group, as defined above. In one embodiment an alkylene refers to a disubstituted alkane, i.e., an alkane substituted at two positions with substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. That is, in one embodiment, a “substituted alkyl” is an “alkylene”.

The term “alkylthio” as used herein refers to alkyl-S—.

The term “alkynyl” as used herein means a straight or branched chain hydrocarbon radical containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “amino” is a term of art and as used herein refers to both unsubstituted and substituted amines, e.g., a moiety that may be represented by the general formulas:

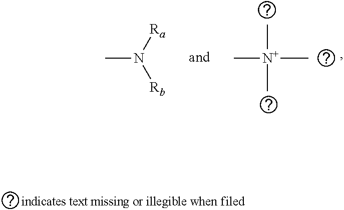

wherein Rₐ, R_{b}, and R_{c} each independently represent a hydrogen, an alkyl, an alkenyl, —(CH₂)ₓ—Rₐ, or Rₐ and R_{b}, taken together with the N atom to which they are attached complete a heterocycle having from 4 to 8 atoms in the ring structure; Rd represents an aryl, a cycloalkyl, a cycloalkenyl, a heterocyclyl or a polycyclyl; and x is zero or an integer in the range of 1 to 8. In certain embodiments, only one of Rₐ or R_{b} may be a carbonyl, e.g., Rₐ, R_{b}, and the nitrogen together do not form an imide. In other embodiments, Rₐ and R_{b} ach independently represent a hydrogen, an alkyl, an alkenyl, or —(CH₂)ₓ—R_{d}. In one embodiment, the term “amino” refers to —NH₂.

The term “aminoacyl” is a term of art and as used herein refers to an acyl group substituted with one or more amino groups.

The term “aminoalkyl” as used herein refers to an alkyl group substituted with one or more one amino groups. In one embodiment, the term “aminoalkyl” refers to an aminomethyl group.

The term “aminothionyl” as used herein refers to an analog of an aminoacyl in which the O of RC(O)— has been replaced by sulfur, hence is of the form RC(S)—.

The term “aralkyl” or “arylalkyl” is a term of art and as used herein refers to an alkyl group substituted with an aryl group.

The term “aryl” is a term of art and as used herein refers to includes monocyclic, bicyclic and polycyclic aromatic hydrocarbon groups, for example, benzene, naphthalene, anthracene, and pyrene. The aromatic ring may be substituted at one or more ring positions with one or more substituents, such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “aryl” also includes polycyclic ring systems having two or more cyclic rings in which two or more carbons are common to two adjoining rings (the rings are “fused rings”) wherein at least one of the rings is an aromatic hydrocarbon, e.g., the other cyclic rings may be cycloalkyls, cycloalkenyls, cycloalkynyls, aryls, heteroaryls, and/or heterocyclyls. In one embodiment, the term “aryl” refers to a phenyl group.

The term “aryloxy” as used herein means an aryl group, as defined herein, appended to the parent molecular moiety through an oxygen atom.

The term “carbocyclyl” as used herein means a monocyclic or multicyclic (e.g., bicyclic, tricyclic, etc.) hydrocarbon radical containing from 3 to 12 carbon atoms that is completely saturated or has one or more unsaturated bonds, and for the avoidance of doubt, the degree of unsaturation does not result in an aromatic ring system (e.g., phenyl). Examples of carbocyclyl groups include 1-cyclopropyl, 1-cyclobutyl, 2-cyclopentyl, 1-cyclopentenyl, 3-cyclohexyl, 1-cyclohexenyl and 2-cyclopentenylmethyl.

The term “carbonyl” as used herein refers to —C(O)—.

The terms “carrier” and “pharmaceutically acceptable carrier” as used herein refer to a diluent, adjuvant, excipient, or vehicle with which a compound is administered or formulated for administration. Non-limiting examples of such pharmaceutically acceptable carriers include liquids, such as water, saline, and oils; and solids, such as gum acacia, gelatin, starch paste, talc, keratin, colloidal silica, urea, and the like. In addition, auxiliary, stabilizing, thickening, lubricating, flavoring, and coloring agents may be used. Other examples of suitable pharmaceutical carriers are described in Remington's Pharmaceutical Sciences by E. W. Martin, herein incorporated by reference in its entirety.

The term “chelator” as used herein means small molecules that bind very tightly to metal ions.

The term “cyano” is a term of art and as used herein refers to —CN.

The term “cycloalkylalkyl” as used herein refers to an alkyl group substituted with one or more cycloalkyl groups.

The term “effective amount” as used herein refers to an amount that is sufficient to bring about a desired biological effect.

The term “ferroportin” (FPN1) as used herein refers to the only known cellular iron exporter. It facilitates the export of iron (Ferrous) from storage cells and absorptive cells to the blood including hepatocytes, macrophages in the liver and spleen, and enterocytes.

The term “fluoroalkyl” as used herein refers to an alkyl group, as defined herein, wherein some or all of the hydrogens are replaced with fluorines.

The term “halo” is a term of art and as used herein refers to —F, —Cl, —Br, or —I.

The term “heteroalkyl group” means a group formed by removing a hydrogen from a carbon of a heteroalkane, where a heteroalkane is an acyclic or cyclic compound consisting entirely of hydrogen atoms, saturated carbon atoms, and one or more heteroatoms. A heteroalkyl group may include one or more substituent groups.

The term “heteroaralkyl” or “heteroarylalkyl” is a term of art and as used herein refers to an alkyl group substituted with a heteroaryl group.

The term “heteroaryl” is a term of art and as used herein refers to a monocyclic, bicyclic, and polycyclic aromatic group having one or more heteroatoms in the ring structure, for example, pyrrole, furan, thiophene, imidazole, oxazole, thiazole, triazole, pyrazole, pyridine, pyrazine, pyridazine and pyrimidine, and the like. The “heteroaryl” may be substituted at one or more ring positions with one or more substituents such as halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, fluoroalkyl (such as trifluromethyl), cyano, or the like. The term “heteroaryl” also includes polycyclic ring systems having two or more cyclic rings in which two or more carbons are common to two adjoining rings (the rings are “fused rings”) wherein at least one of the rings is an aromatic group having one or more heteroatoms in the ring structure, e.g., the other cyclic rings may be cycloalkyls, cycloalkenyls, cycloalkynyls, aryls, heteroaryls, and/or heterocyclyls.

The term “heteroaryloxy” as used herein means a heteroaryl group, as defined herein, appended to the parent molecular moiety through an oxygen atom.

The term “heteroatom” is art-recognized, and includes an atom of any element other than carbon or hydrogen. Illustrative heteroatoms include boron, nitrogen, oxygen, phosphorus, sulfur and selenium, and alternatively oxygen, nitrogen or sulfur.

The term “heterocyclyl” as used herein refers to a radical of a non-aromatic ring system, including, but not limited to, monocyclic, bicyclic, and tricyclic rings, which can be completely saturated or which can contain one or more units of unsaturation, for the avoidance of doubt, the degree of unsaturation does not result in an aromatic ring system, and having 3 to 12 atoms including at least one heteroatom, such as nitrogen, oxygen, or sulfur. For purposes of exemplification, which should not be construed as limiting the scope of this disclosure, the following are examples of heterocyclic rings: aziridinyl, azirinyl, oxiranyl, thiiranyl, thiirenyl, dioxiranyl, diazirinyl, azetyl, oxetanyl, oxetyl, thietanyl, thietyl, diazetidinyl, dioxetanyl, dioxetenyl, dithietanyl, dithietyl, furyl, dioxalanyl, pyrrolyl, oxazolyl, thiazolyl, imidazolyl, oxadiazolyl, thiadiazolyl, triazolyl, triazinyl, isothiazolyl, isoxazolyl, thiophenyl, pyrazolyl, tetrazolyl, pyridyl, pyridazinyl, pyrimidinyl, pyrazinyl, triazinyl, tetrazinyl, quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl, naphthyridinyl, azepines, azetidinyl, morpholinyl, oxopiperidinyl, oxopyrrolidinyl, piperazinyl, piperidinyl, pyrrolidinyl, quinicludinyl, thiomorpholinyl, tetrahydropyranyl and tetrahydrofuranyl.

The term “heterocycloalkylalkyl” as used herein refers to an alkyl group substituted with one or more heterocycloalkyl (i.e., heterocyclyl) groups.

The term “hydroxy” is a term of art and as used herein refers to —OH.

The term “inhibit” as used herein means decrease by an objectively measurable amount or extent. In various embodiments, “inhibit” means decrease by at least 5, 10, 20, 30, 40, 50, 60, 70, 80, 90, or 95 percent compared to relevant control. In one embodiment, “inhibit” means decrease 100 percent, i.e., halt or eliminate.

As used herein the expression “neurodegeneration with brain iron accumulation” or “NBIA” has its general meaning in the art and refers to a group of rare, genetic neurological disorders characterized by abnormal accumulation of iron in the basal ganglia. The hallmark clinical manifestations of NBIA relate to the body's muscle function and feature a progressive movement disorder, including dystonia, choreoathetosis, stiffness in the arms and legs and Parkinsonism. Most forms of NBIA involve eye disease. The most common problems are degeneration of the retina and optic atrophy. A general loss of brain cells and tissue also are frequently observed, conditions called cerebral atrophy and cerebellar atrophy. Onset of NBIA ranges from infancy to adulthood. Progression can be rapid or slow with long periods of stability. In some embodiments, the NBIA results from a disease gene selected from PANK2, PLA2G6, COASY, FA2H, ATP13A2, C2orf37, WDR45, C19ORFf12, CP, FTL, GTPBP2, CRAT and REPS1. In some embodiments, the NBIA is a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA. A sub-type of NBIA are “beta-propeller protein associated neurodegeneration” and Pantothenate Kinase-associated Neurodegeneration (PKAN).

The term “pharmaceutically acceptable salt” as used herein includes salts derived from inorganic or organic acids including, for example, hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2-sulfonic, and other acids. Pharmaceutically acceptable salt forms can include forms wherein the ratio of molecules comprising the salt is not 1:1. For example, the salt may comprise more than one inorganic or organic acid molecule per molecule of base, such as two hydrochloric acid molecules per molecule of compound of Formula Ia or Ib. As another example, the salt may comprise less than one inorganic or organic acid molecule per molecule of base, such as two molecules of compound of Formula Ia or Ib per molecule of tartaric acid.

The term “subject” as used herein refers to a mammal. In various embodiments, a subject is a mammal and includes a mouse, rat, rabbit, cat, dog, pig, sheep, horse, cow, or non-human primate. In one embodiment, a subject is a human.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, fragmentation, decomposition, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents may be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. This disclosure is not intended to be limited in any manner by the permissible substituents of organic compounds.

The term “therapeutically effective amount” as used herein refers to an amount that is sufficient to bring about a desired therapeutic effect.

The term “thiocarbonyl” as used herein refers to —C(S)—.

The term “treat” as used herein means prevent, halt or slow the progression of, or eliminate a disease or condition in a subject. In one embodiment, “treat” means halt or slow the progression of, or eliminate a disease or condition in a subject. In one embodiment, “treat” means reduce at least one objective manifestation of a disease or condition in a subject.

Certain compounds contained in compositions of the present disclosure may exist in particular geometric or stereoisomeric forms. In addition, compounds of the present disclosure may also be optically active. The present disclosure contemplates all such compounds, including cis- and trans-isomers, (R)- and (S)-enantiomers, diastereoisomers, (D)-isomers, (L)-isomers, the racemic mixtures thereof, and other mixtures thereof, as falling within the scope of the present disclosure. Additional asymmetric carbon atoms may be present in a substituent such as an alkyl group. All such isomers, as well as mixtures thereof, are intended to be included in this disclosure.

If, for instance, a particular enantiomer of compound of the present disclosure is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

For purposes of the present disclosure, the chemical elements are identified in accordance with the Periodic Table of the Elements, CAS version, Handbook of Chemistry and Physics, 67th Ed., 1986-87, inside cover.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference). Unless otherwise defined, all technical and scientific terms used herein have the same meaning as commonly understood by one of ordinary skill in the art to which this disclosure pertains.

### Compounds

In some aspects, the present disclosure provides a compound or a tautomer thereof, or a pharmaceutically acceptable salt of either, represented by Formula (I), (Ia), (Ib), (Ic), (Id), (IIa), (IIb), or (IIc):

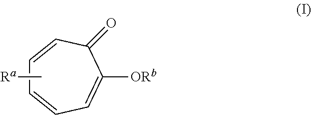

R^{a} is C₁₋₂₀-alkyl, C₂₋₂₀-alkenyl, C₂₋₂₀-alkynyl, C₃₋₉-cycloalkyl, aryl, or heteroaryl, each of which is unsubstituted or substituted with a substituent selected from the group consisting of halo, NO₂, CN, C₁₋₆-alkyl, C₁₋₆-haloalkyl, and C₁₋₆-alkoxy; and
R^{b} is hydrogen or methyl; provided the compound is not hinokitiol;

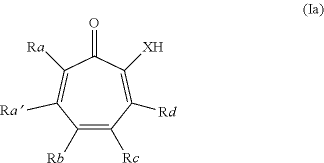

X represents oxygen or sulfur;
Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
Rₐ′ represents hydrogen, halo, alkyl, or substituted alkyl; and R_{b}, R_{c}, and R_{d} are independently selected from the group consisting of hydrogen, halo, alkyl, substituted alkyl, heteroalkyl, alkylcycloalkyl, alkylheterocycloalkyl, alkoxy, substituted alkoxy, alkoxyalkyl, substituted alkoxyalkyl, cycloalkyloxy, substituted cycloalkyloxy, heterocycloalkyloxy, substituted heterocycloalkyloxy, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl; provided that Rₐ, R_{b}, R_{c}, and R_{d} are not all hydrogen; Rₐ′ is hydrogen, Rₐ′ is halo, or Rₐ′ is alkyl, or substituted alkyl;
   at least one of Rₐ, R_{b}, R_{c}, and R_{d} is selected from the group consisting of halo, alkyl, substituted alkyl, heteroalkyl, alkylcycloalkyl, alkylheterocycloalkyl, alkoxy, substituted alkoxy, alkoxyalkyl, substituted alkoxyalkyl, cycloalkyloxy, substituted cycloalkyloxy, heterocycloalkyloxy, substituted heterocycloalkyloxy, cycloalkyl, substituted cycloalkyl, heterocycloalkyl, and substituted heterocycloalkyl; and
   at least one of Rₐ, Rₐ′, R_{b}, R_{c}, and R_{d} is selected from the group consisting of methyl, ethyl, n-propyl, and isopropyl;

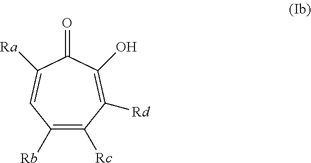

Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;

R_{b}, R_{c}, and R_{d} are independently selected from the group consisting of hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl; provided that Rₐ, R_{b}, R_{c}, and R_{d} are not all hydrogen;

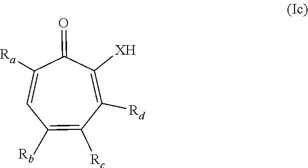

X represents sulfur or oxygen;
Rₐ, R_{b}, R_{c}, and R_{d} independently represent hydrogen, halo, alkyl, substituted alkyl, heteroalkyl, alkoxy, substituted alkoxy, alkoxyalkyl, substituted alkoxyalkyl, aryloxy, substituted aryloxy, heteroaryloxy, substituted heteroaryloxy, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, heteroalkynyl, aryl, substituted aryl, heteroaryl, or substituted heteroaryl; and
   at least one of Rₐ, R_{b}, R_{c}, and R_{d} is aryloxy, substituted aryloxy, heteroaryloxy, substituted heteroaryloxy, aryl, substituted aryl, heteroaryl, substituted heteroaryl; provided that Rₐ, R_{b}, R_{c}, and R_{d} are not all hydrogen;

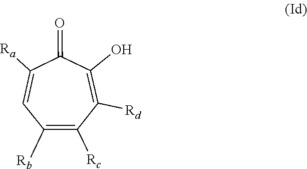

Rₐ, R_{b}, R_{c}, and R_{d} independently represent hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, heteroalkynyl, aryl, substituted aryl, heteroaryl, substituted heteroaryl;
   at least one of R_{b}, R_{c}, and R_{d} is aryl, substituted aryl, heteroaryl, or substituted heteroaryl; and provided that Rₐ, R_{b}, R_{c}, and R_{d} are not all hydrogen; or

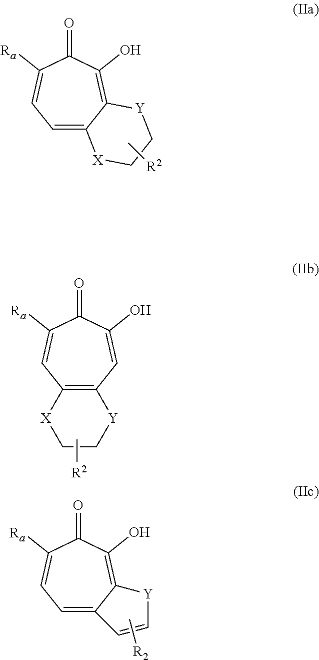

Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
   X and Y independently represent O, S, NH, or CR₅R₆;
   R₂ represents —F, alkyl, haloalkyl, or alkoxy; and
   R₅ and R₆ represent independently for each occurrence H, (C1-C15) alkyl, or substituted (C1-C15) alkyl;
   provided the compound is not

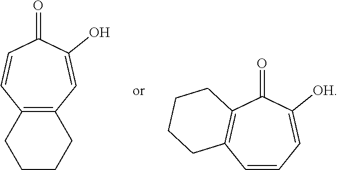

In an embodiment, the compound is selected from:

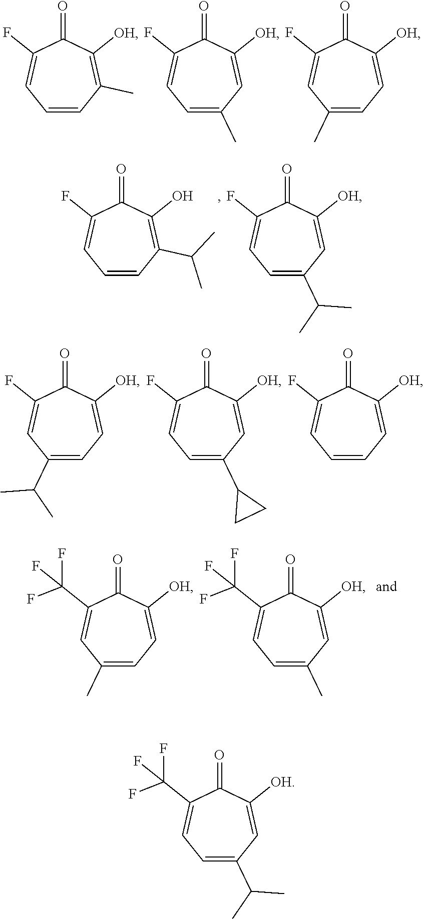

In an embodiment, the compound is selected from:

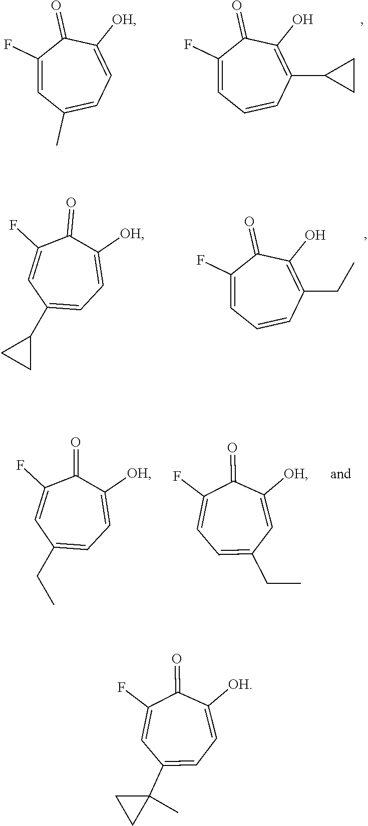

In an embodiment, the compound is selected from:

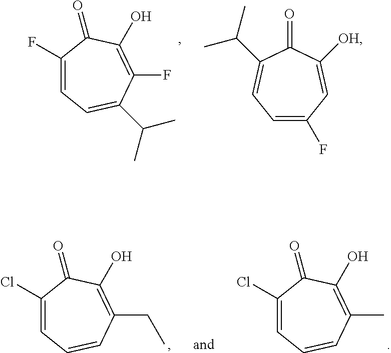

In an embodiment, the compound is selected from:

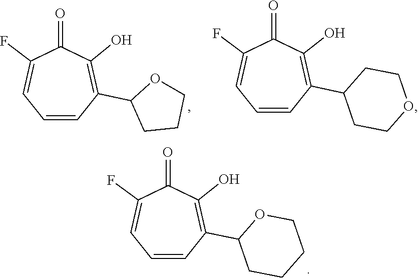

In an embodiment, the compound is selected from:

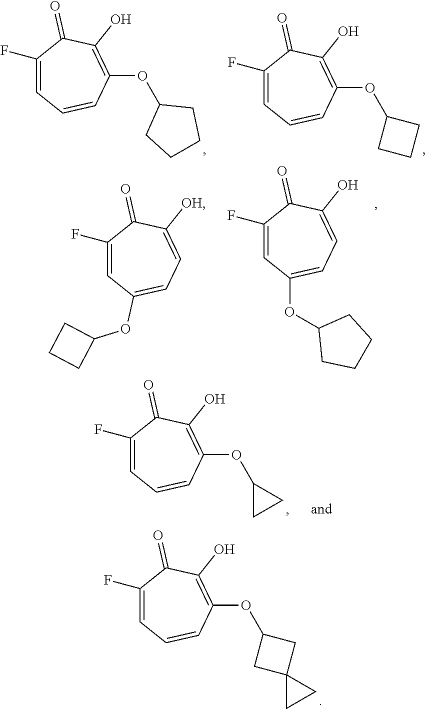

In an embodiment, the compound is selected from:

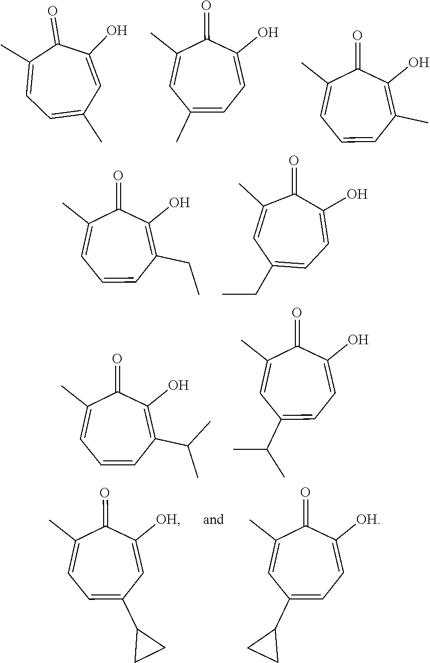

In an embodiment, the compound is selected from:

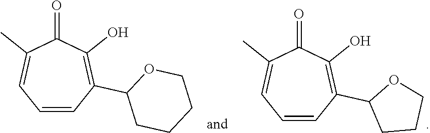 [SMILES: Cc1cccc(C2CCCCO2)c(O)c1=O ; Cc1cccc(C2CCCO2)c(O)c1=O]

In an embodiment, the compound is selected from:

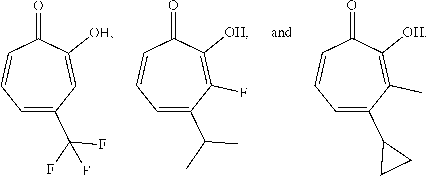

In an embodiment, the compound is selected from:

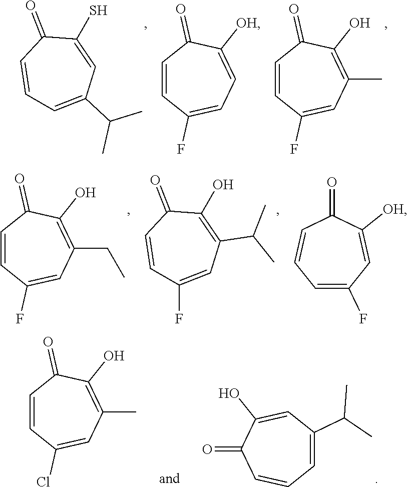

In an embodiment, the compound is selected from:

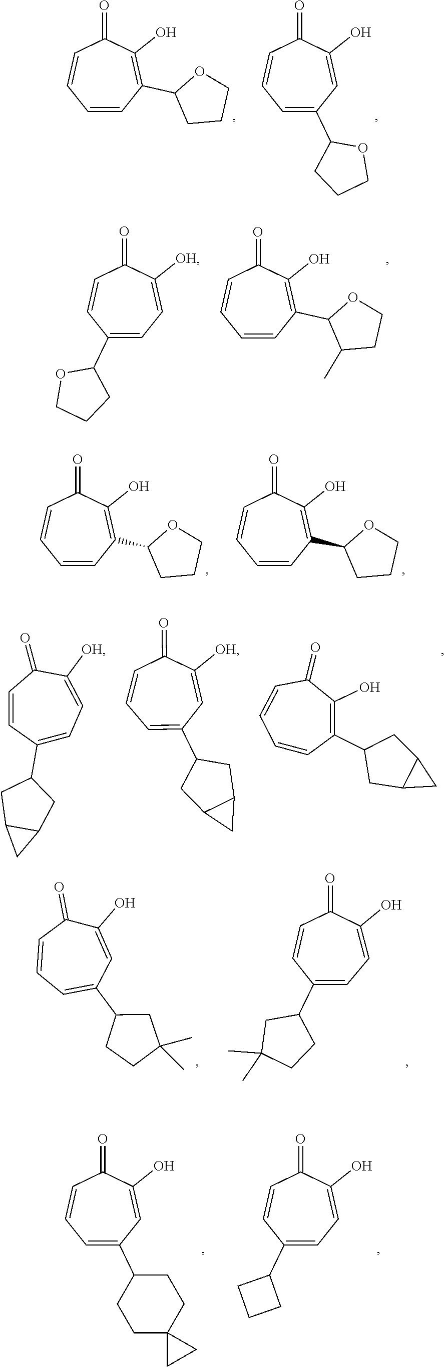

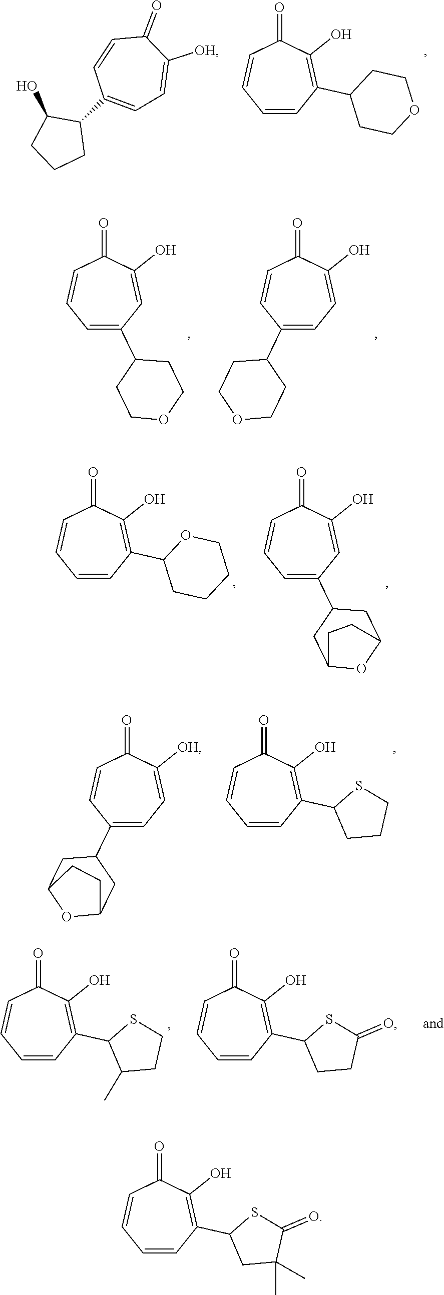

In an embodiment, the compound is selected from:

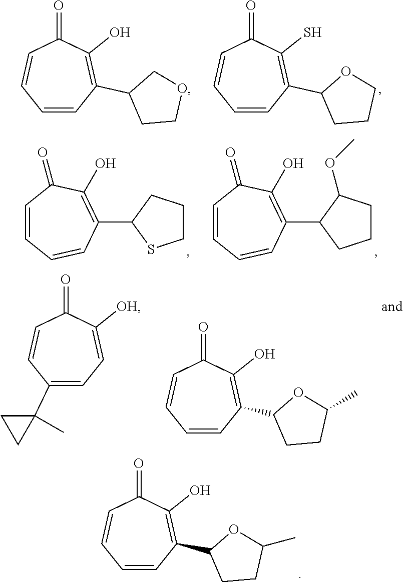

In an embodiment, the compound is selected from:

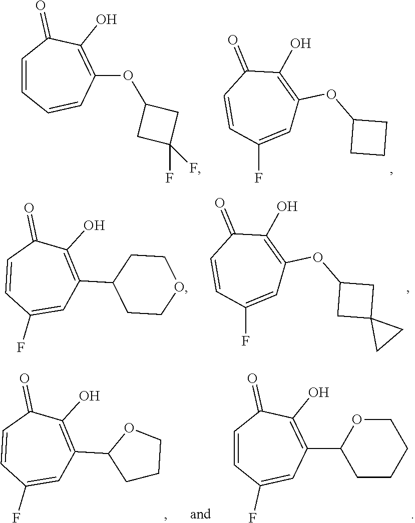

In an embodiment, the compound is selected from:

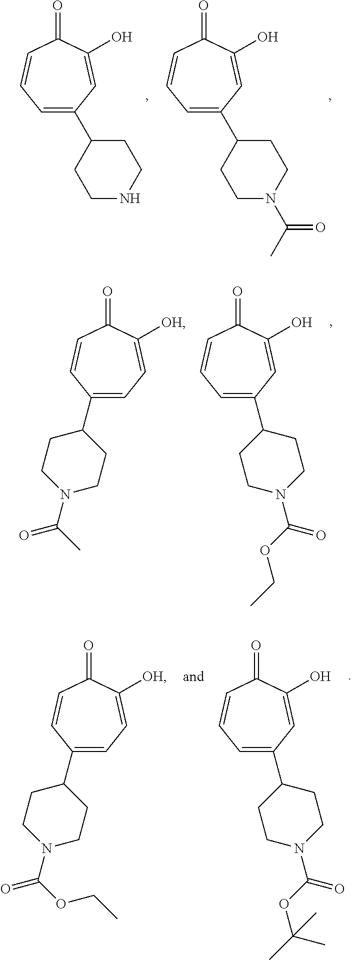

In an embodiment, the compound is selected from:

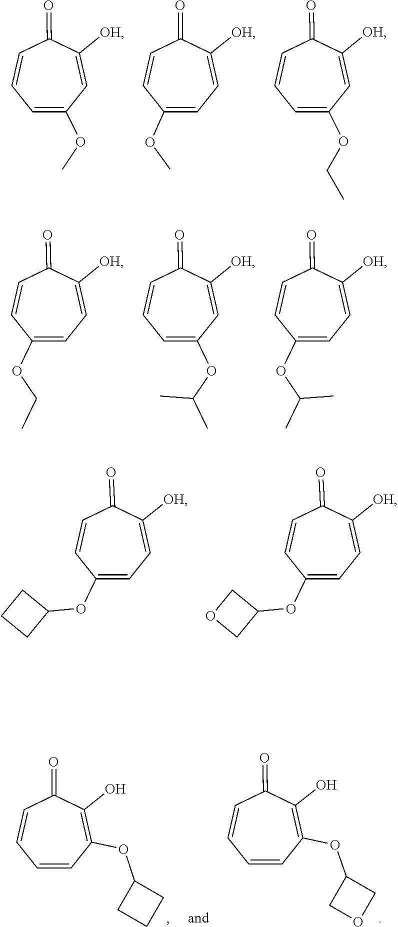

In an embodiment, the compound is selected from:

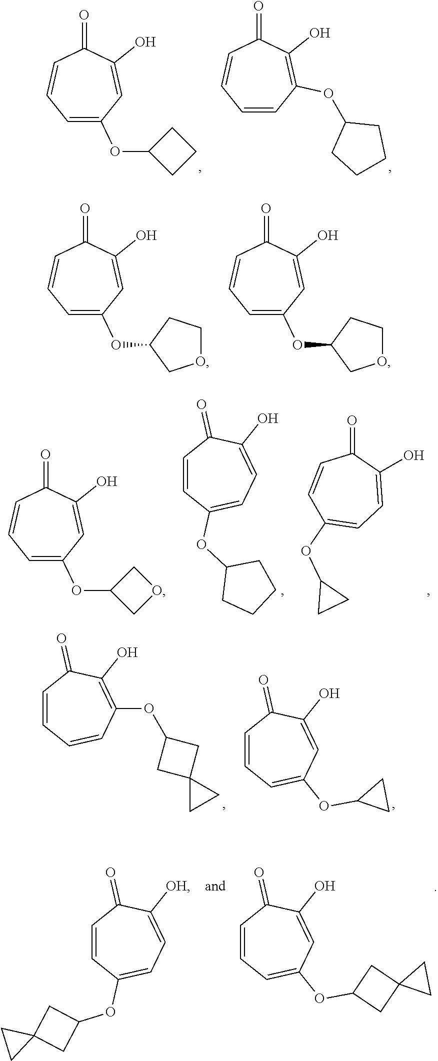

In an embodiment, the compound is selected from:

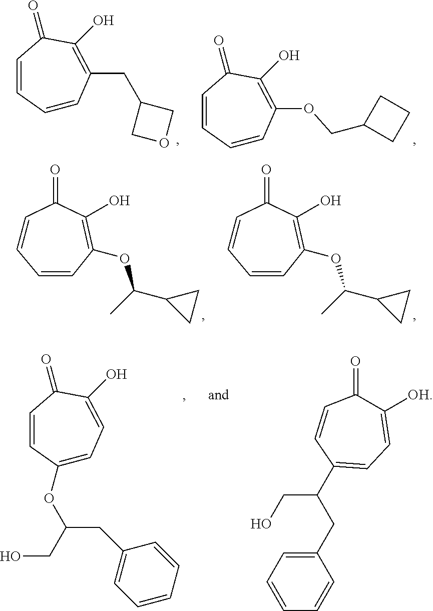

In an embodiment, the compound is selected from:

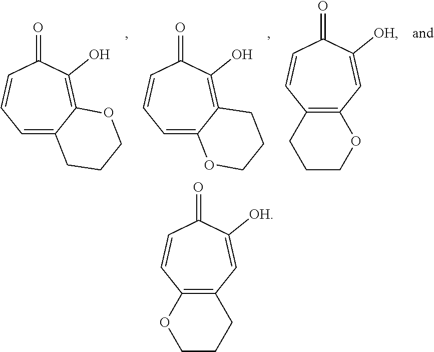

In an embodiment, the compound is selected from:

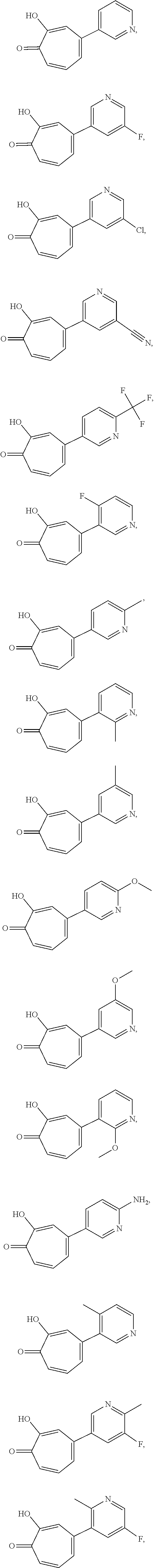

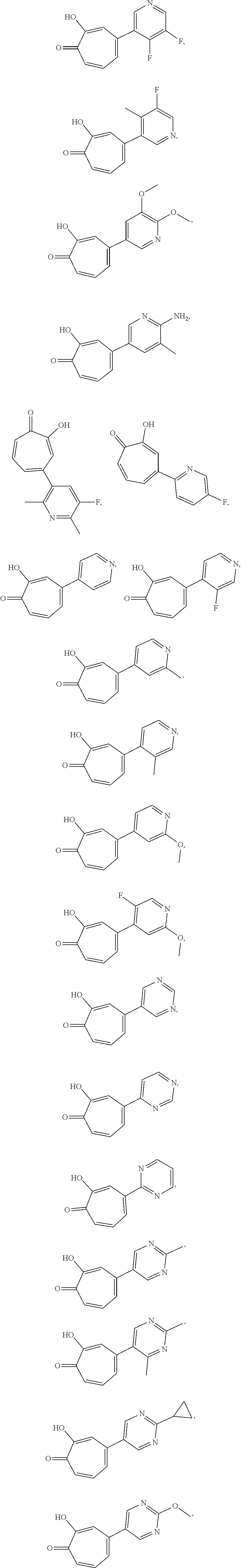

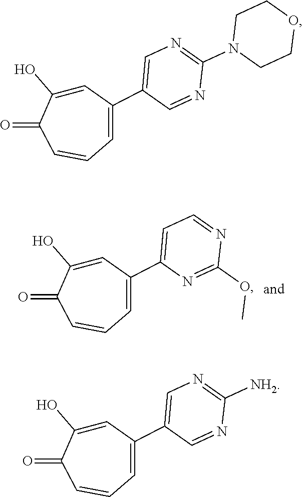 [SMILES: O=c1cccc(-c2cnc(N3CCOCC3)nc2)cc1O ; COc1nccc(-c2cccc(=O)c(O)c2)n1 ; Nc1ncc(-c2cccc(=O)c(O)c2)cn1]

In an embodiment, the compound is selected from:

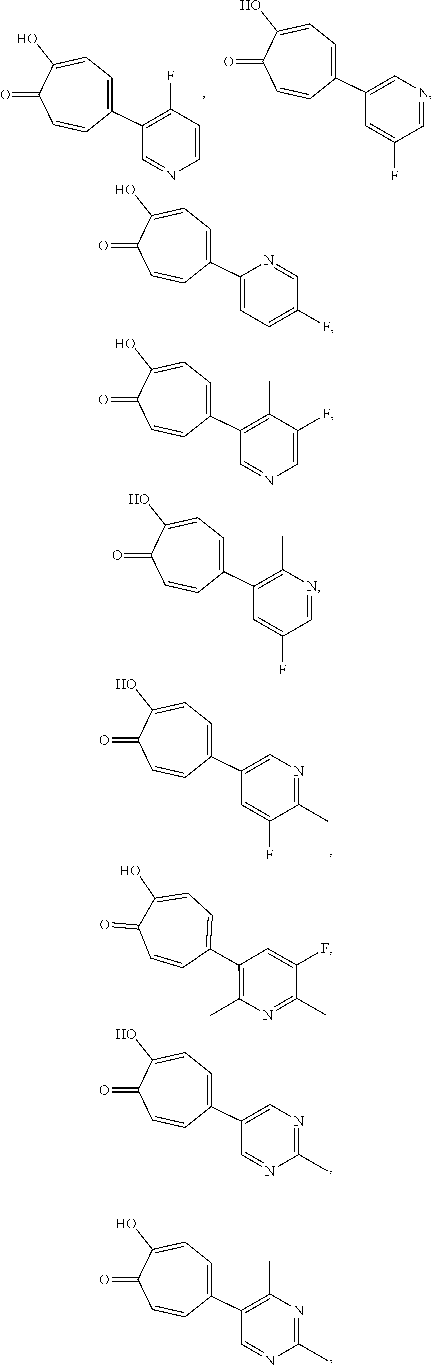

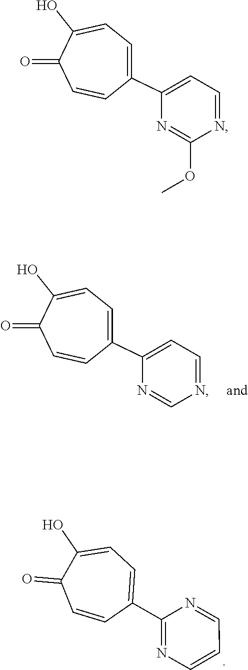 [SMILES: COc1nccc(-c2ccc(O)c(=O)cc2)n1 ; O=c1ccc(-c2ccncn2)ccc1O ; O=c1ccc(-c2ncccn2)ccc1O]

In an embodiment, the compound is selected from:

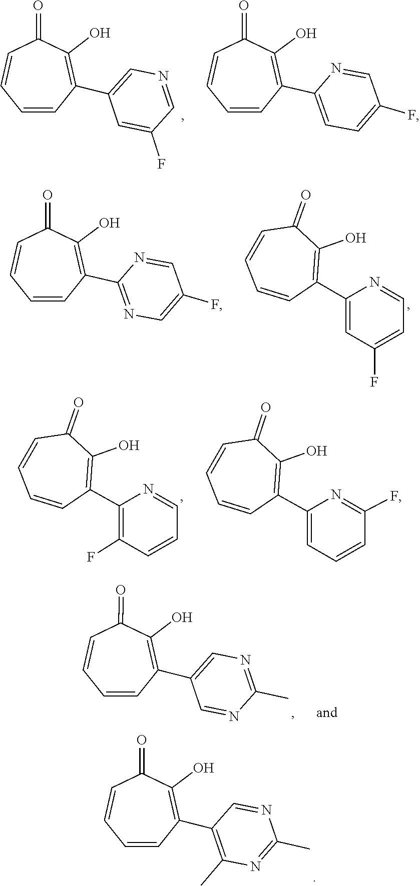

In an embodiment, the compound is selected from:

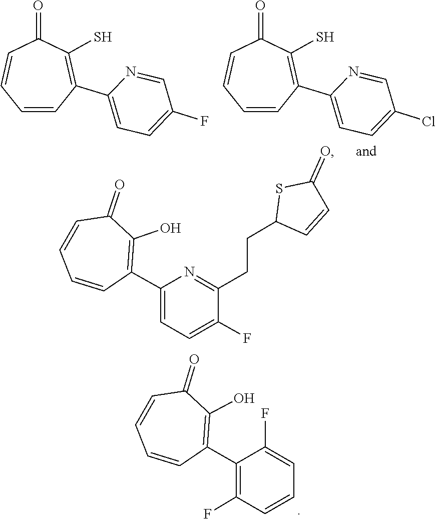

In an embodiment, the compound is selected from:

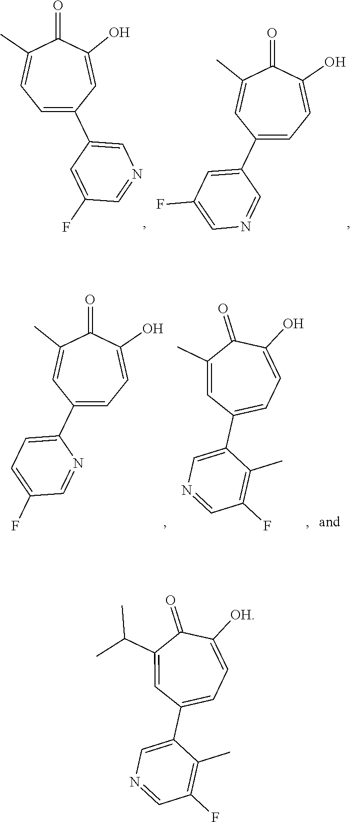

In an embodiment, the compound is selected from:

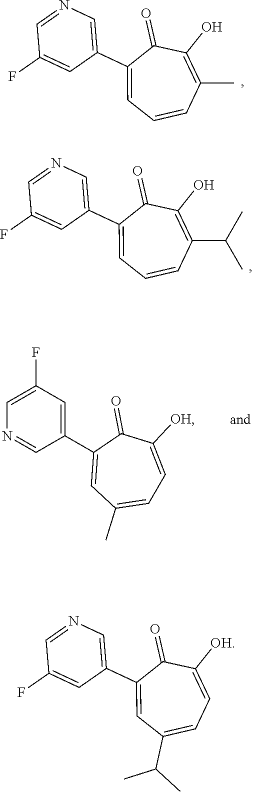 [SMILES: Cc1cccc(-c2cncc(F)c2)c(=O)c1O ; CC(C)c1cccc(-c2cncc(F)c2)c(=O)c1O ; Cc1ccc(O)c(=O)c(-c2cncc(F)c2)c1 ; "and" ; CC(C)c1ccc(O)c(=O)c(-c2cncc(F)c2)c1]

In an embodiment, the compound is selected from:

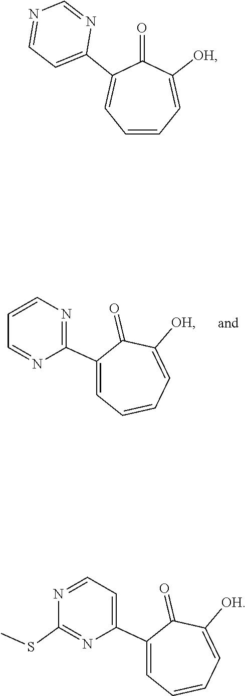 [SMILES: O=c1c(O)ccccc1-c1ccncn1 ; O=c1c(O)ccccc1-c1ncccn1 ; "and" ; CSc1nccc(-c2ccccc(O)c2=O)n1]

In an embodiment, the compound is selected from:

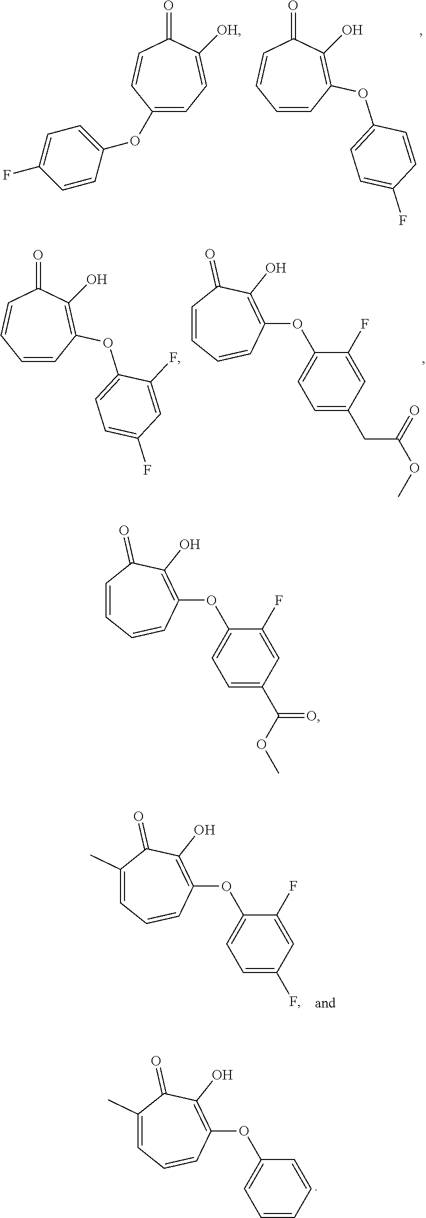

In an embodiment, the compound is selected from:

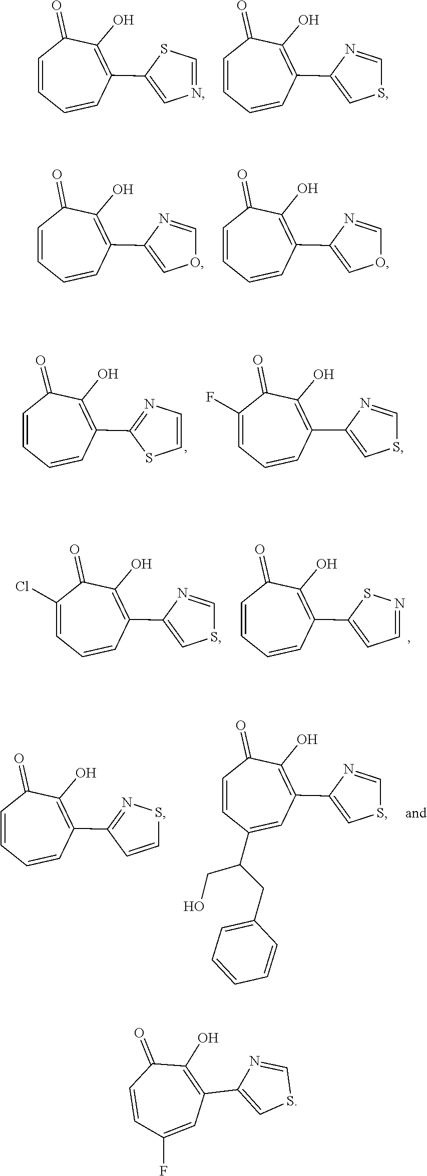

In an embodiment, the compound is selected from:

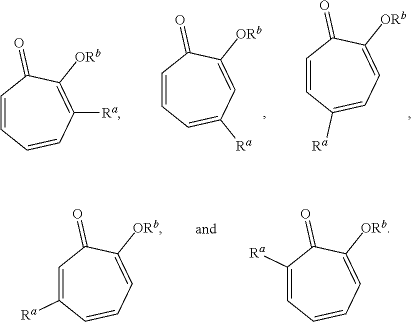

In an embodiment, the compound is selected from:

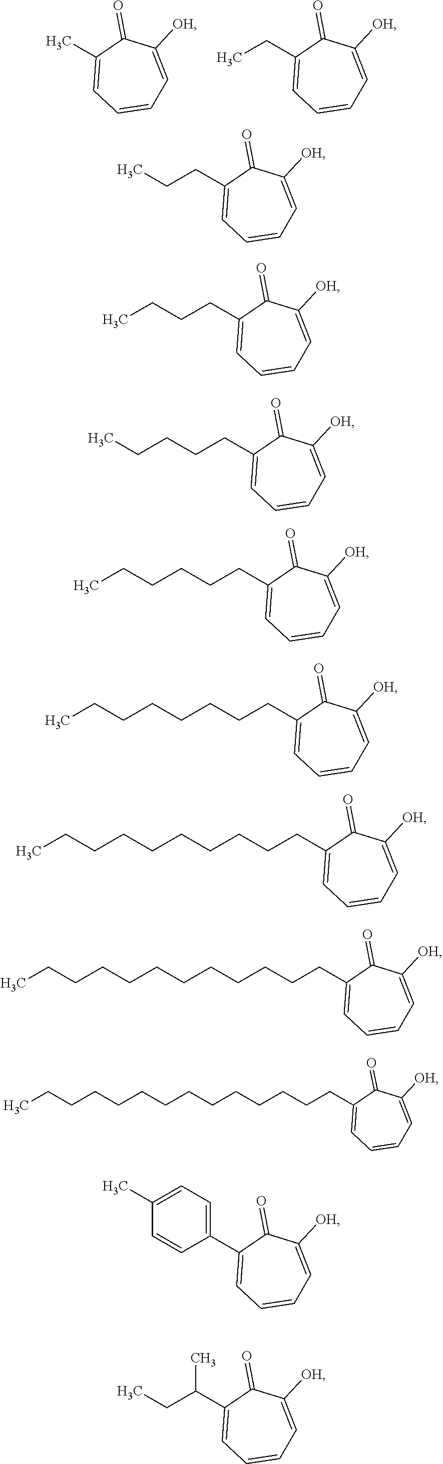

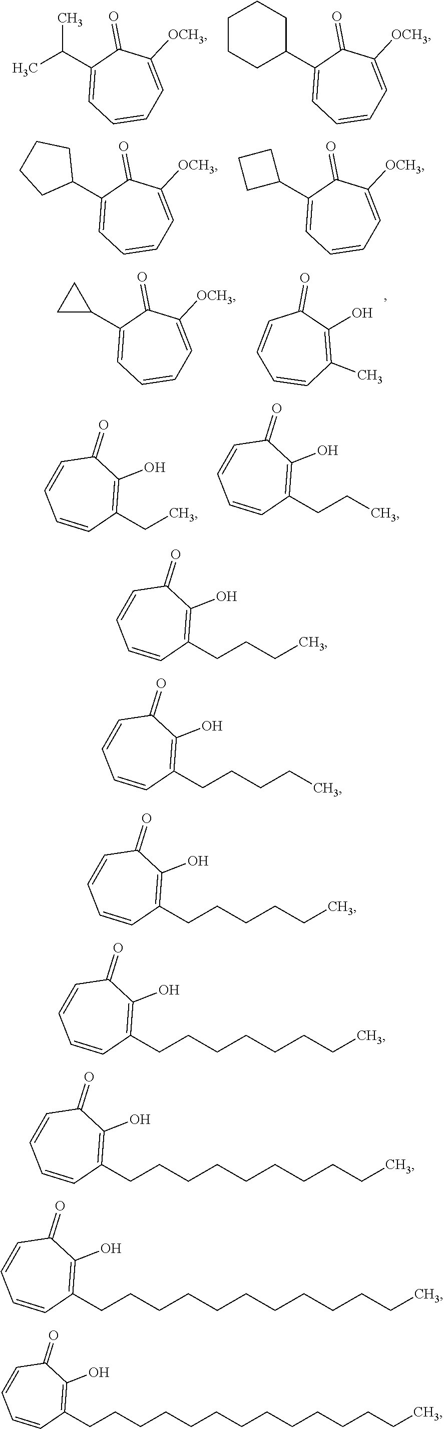

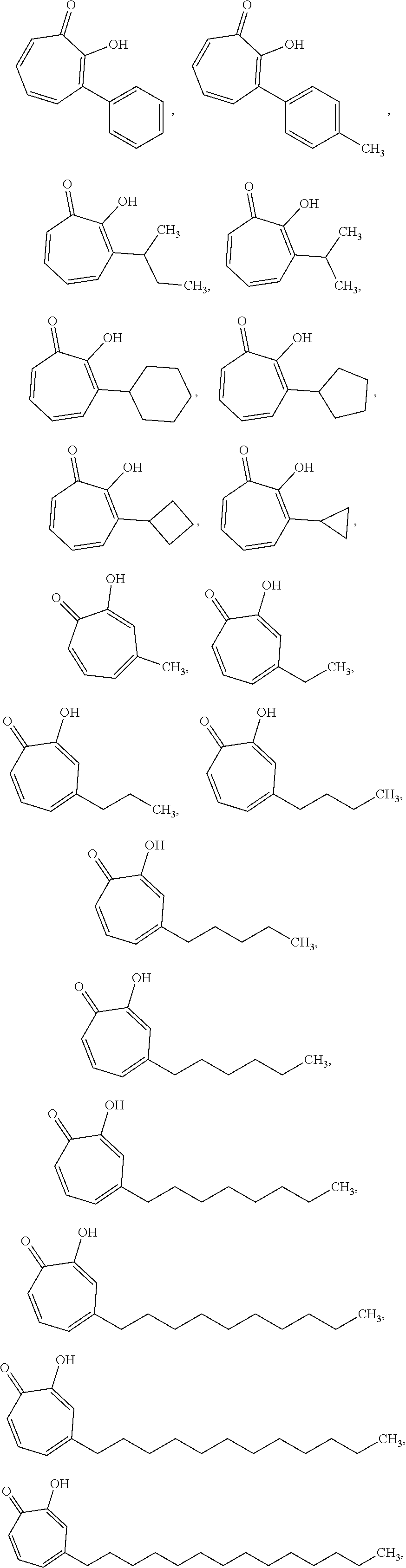

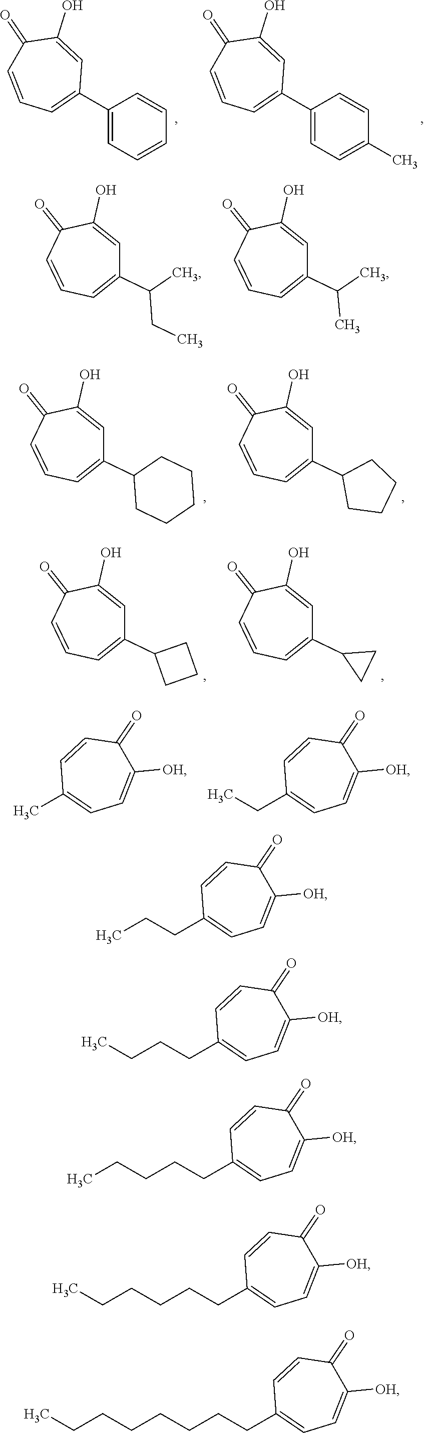

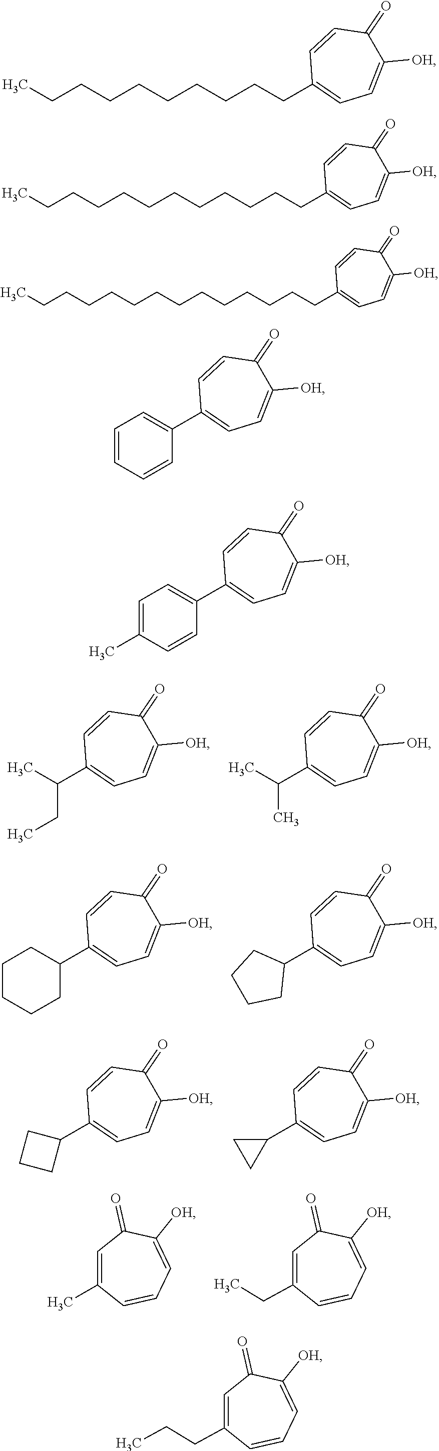

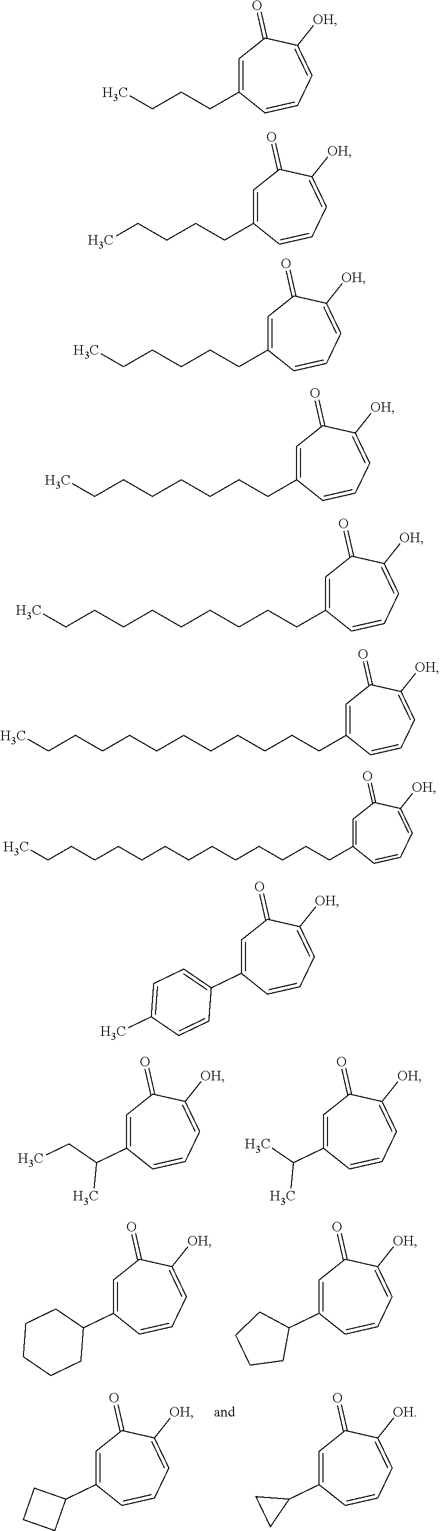

In an embodiment, the compound is selected from:

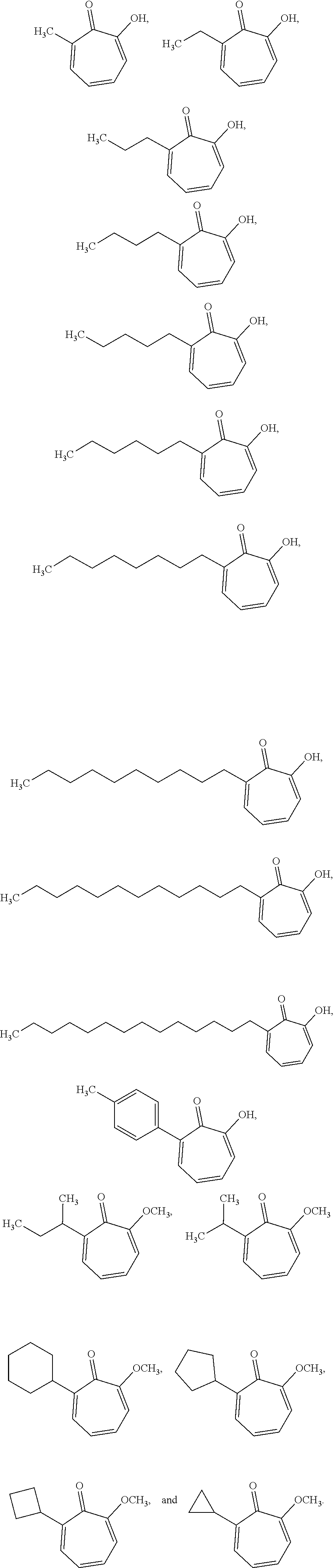

In an embodiment, the compound is selected from:

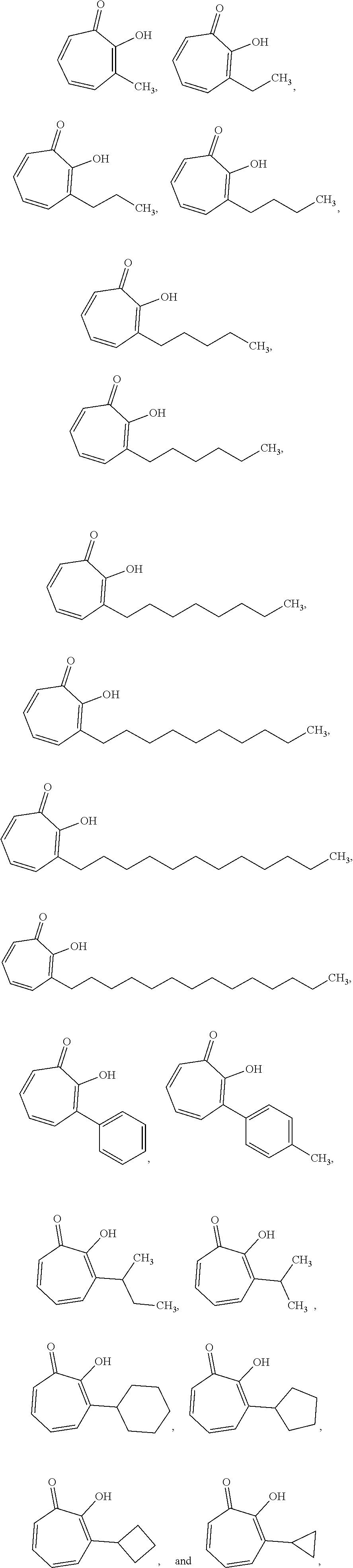

In an embodiment, the compound is selected from:

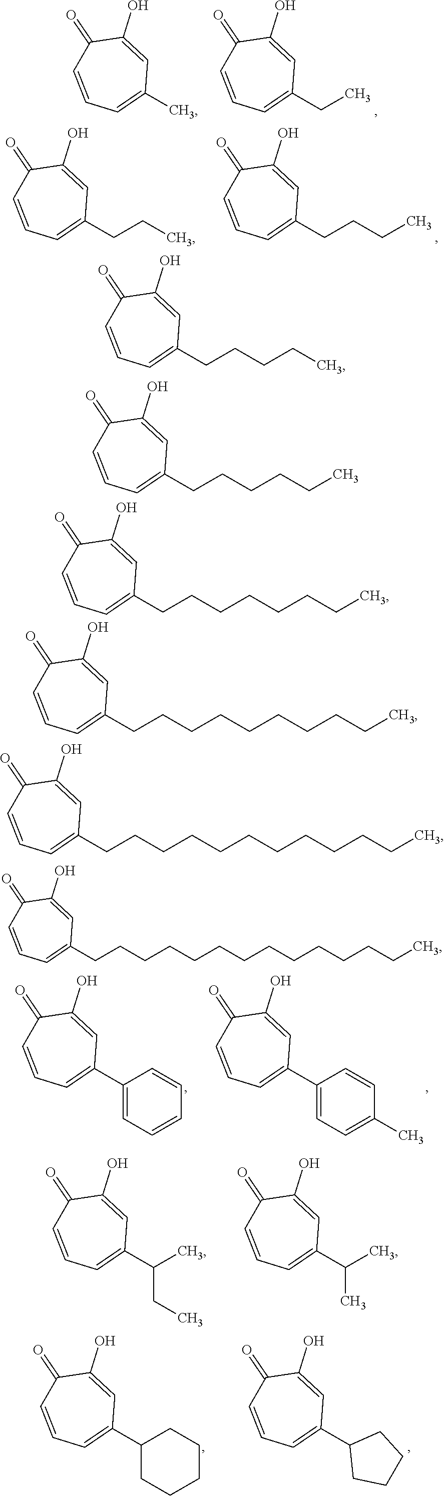

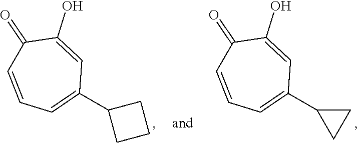 [SMILES: O=c1cccc(C2CCC2)cc1O ; O=c1cccc(C2CC2)cc1O]

In an embodiment, the compound is selected from:

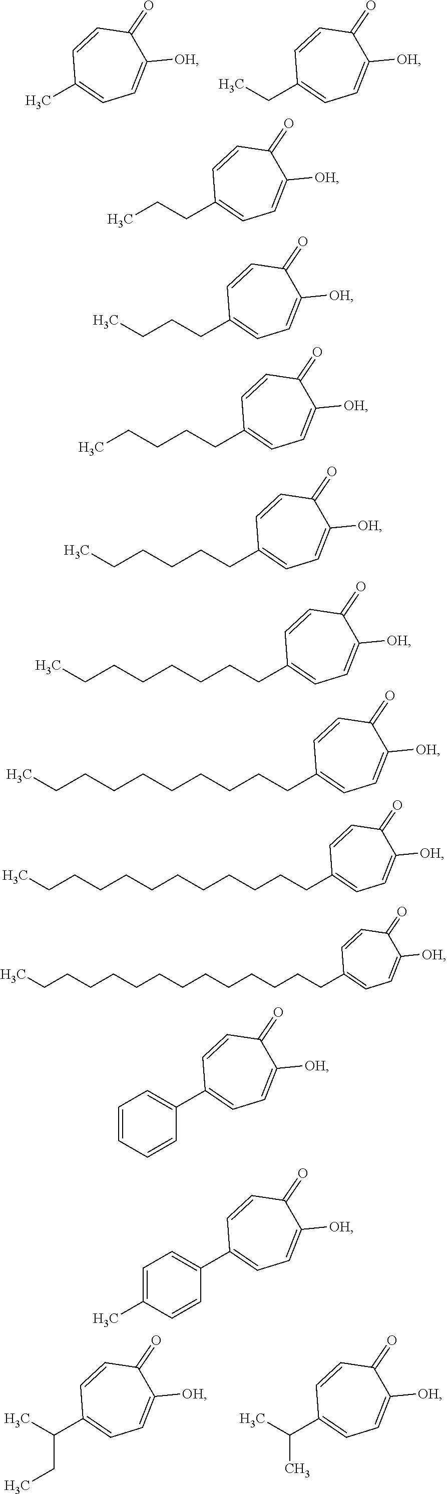

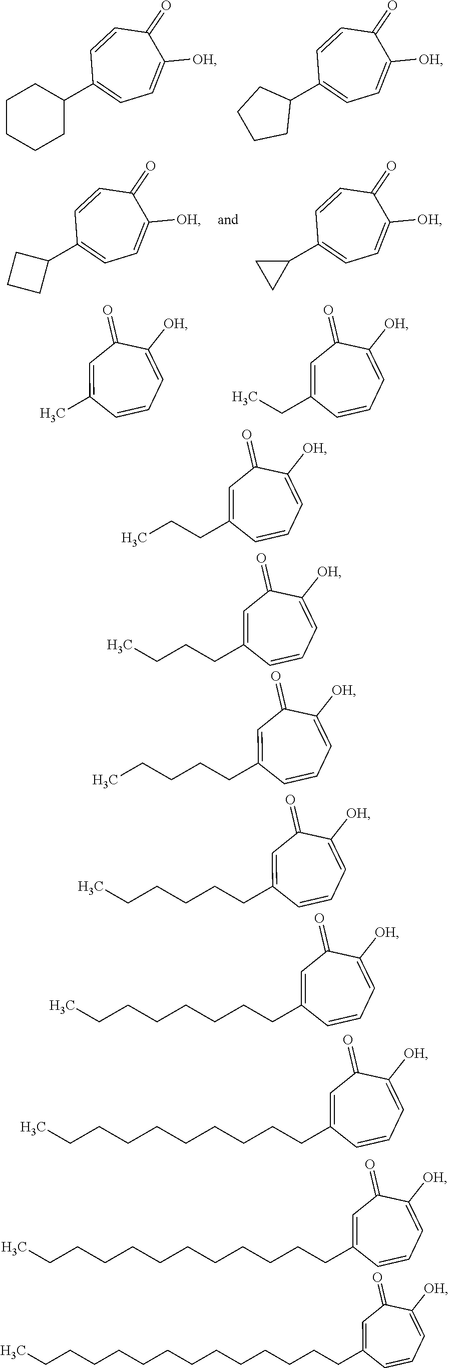

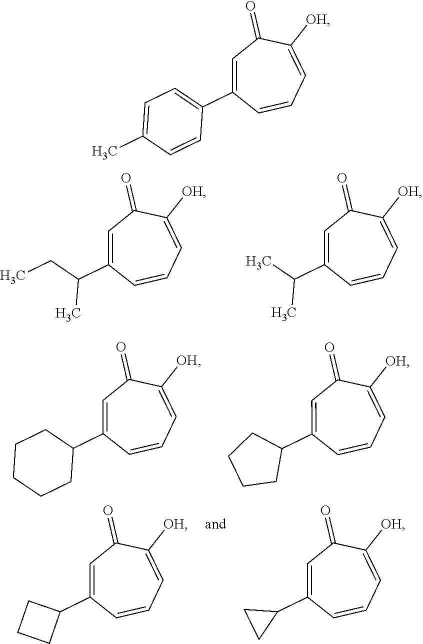

In an embodiment, the compound is selected from:

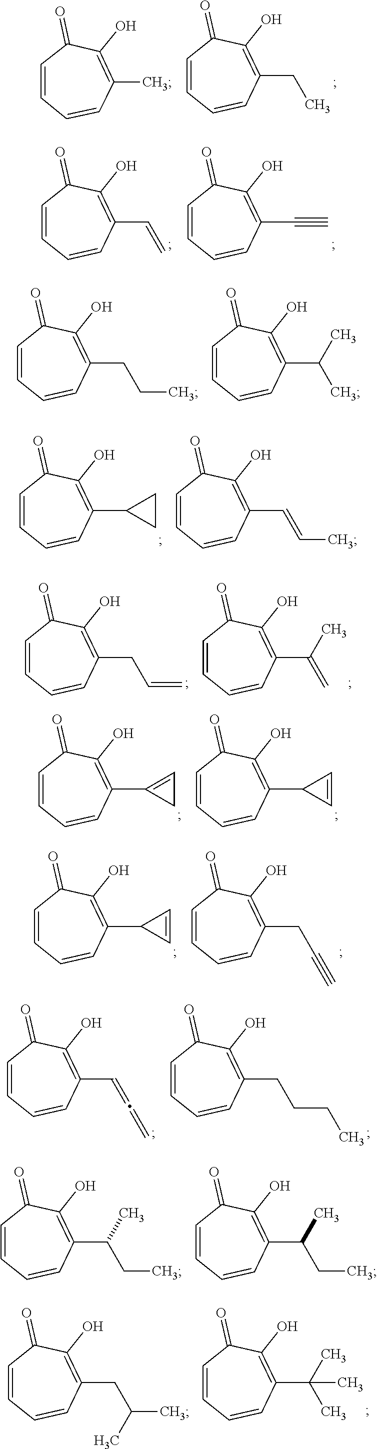

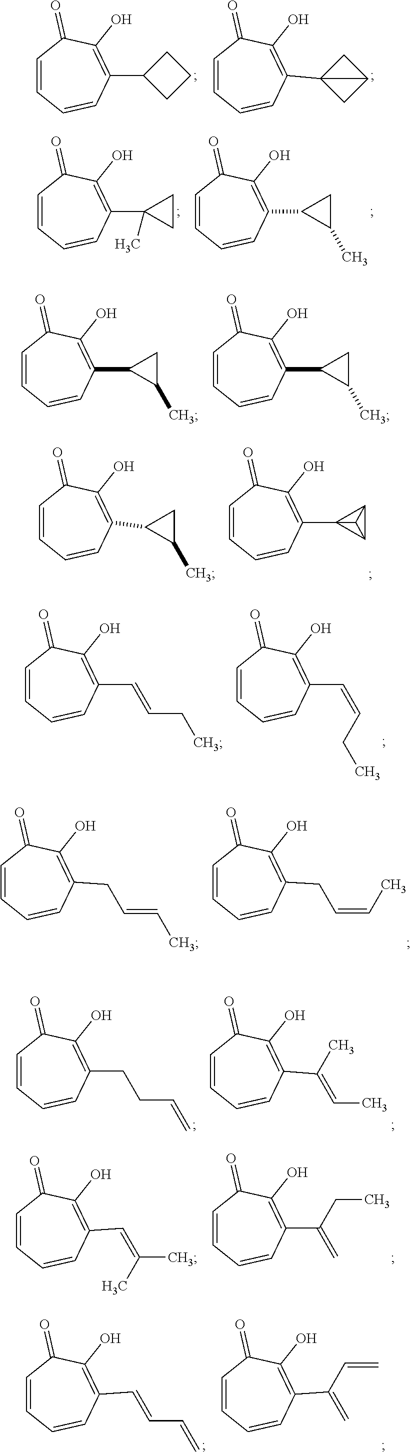

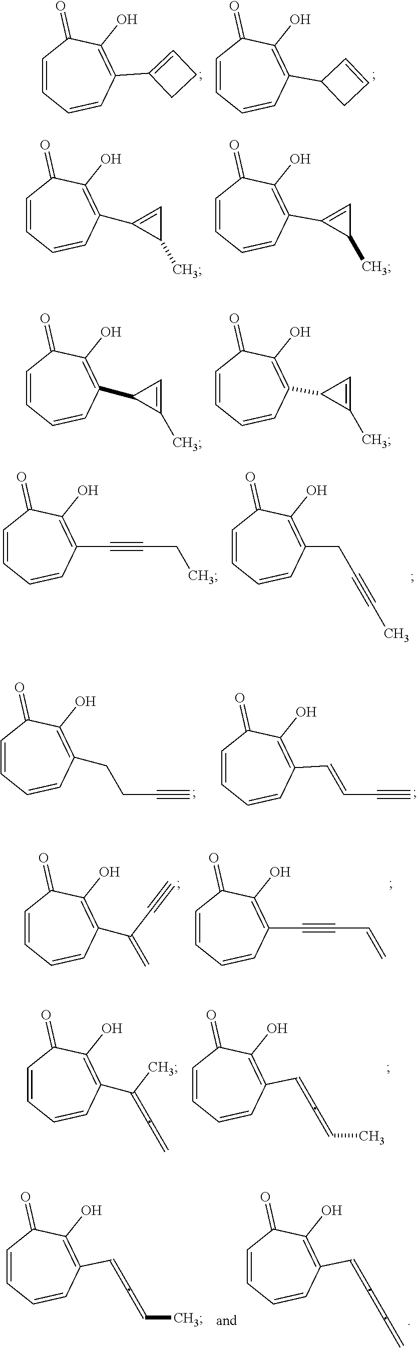

In an embodiment, the compound is selected from:

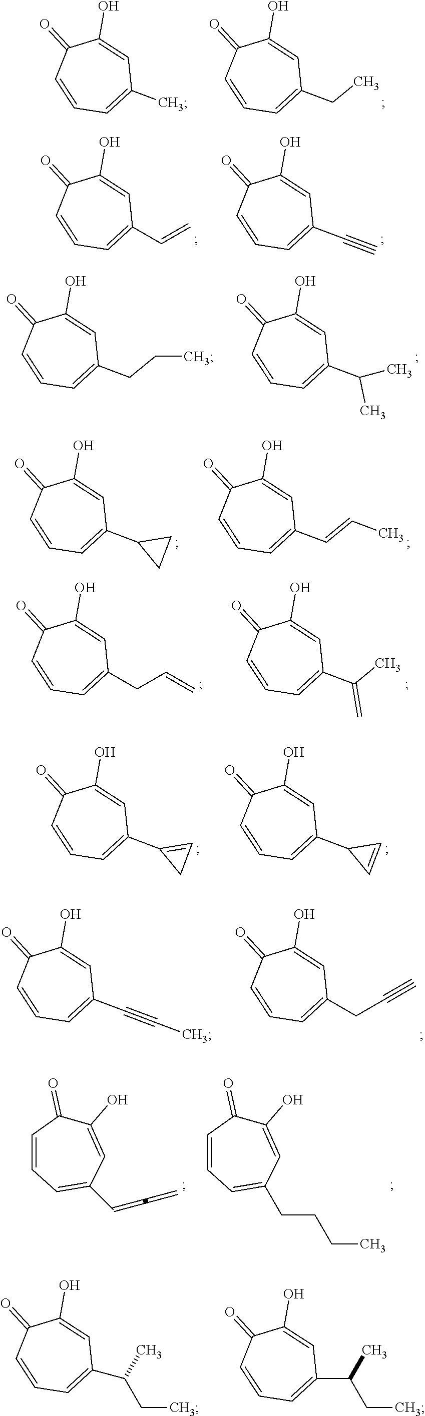

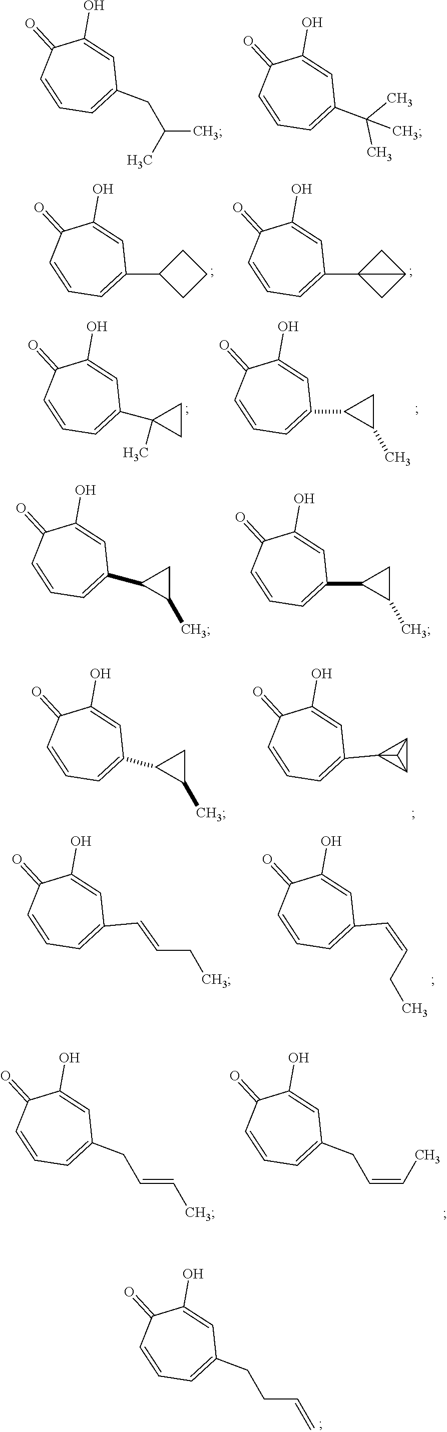

In an embodiment, the compound is selected from:

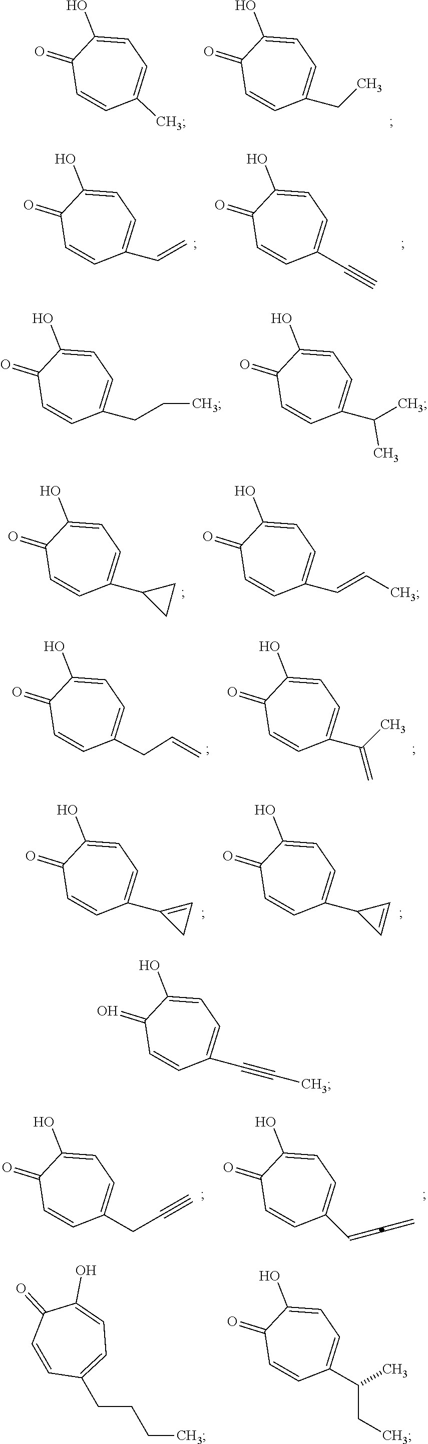

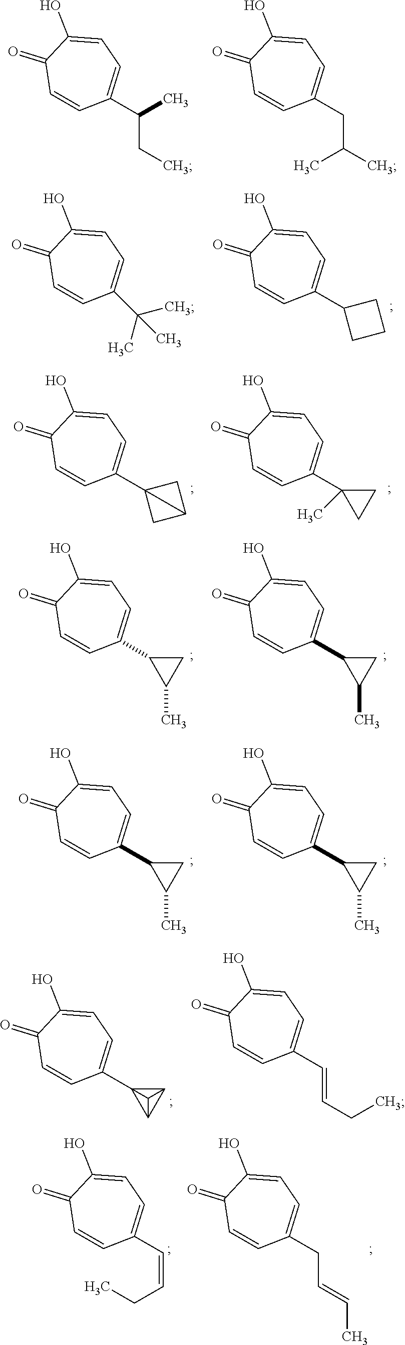

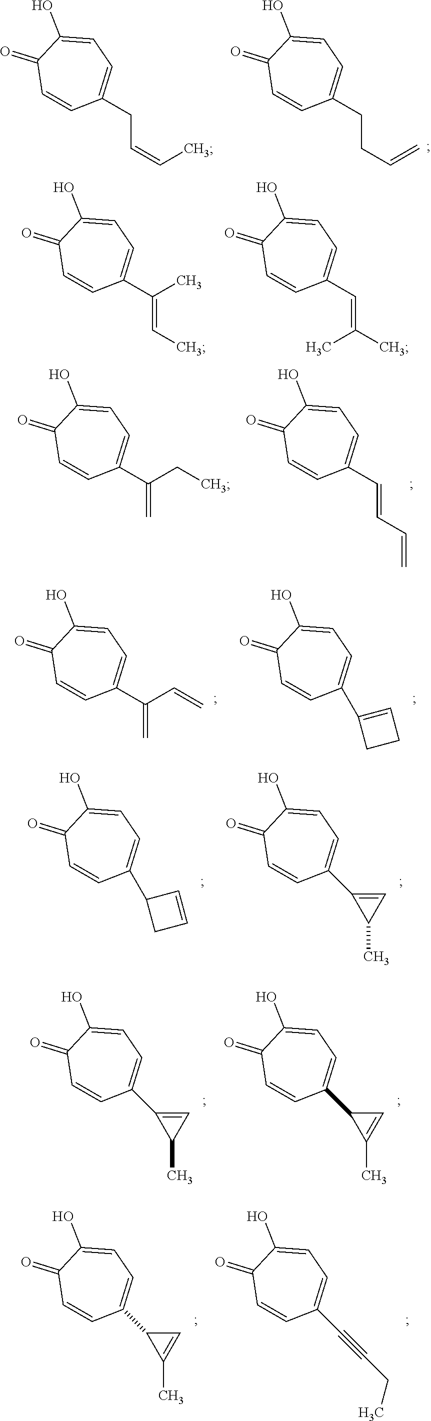

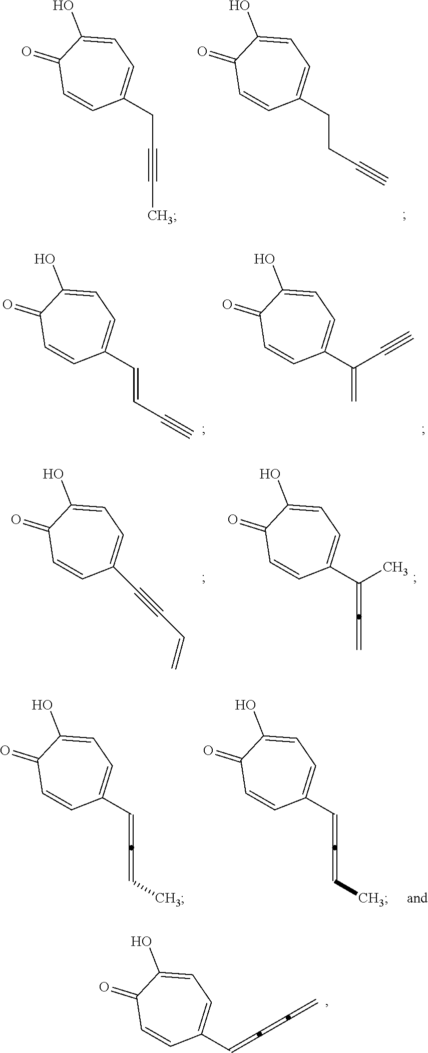

In an embodiment, the compound is

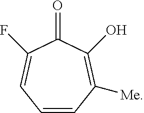 [SMILES: Cc1cccc(F)c(=O)c1O]

In an embodiment, the compound is

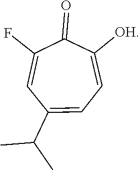

In an embodiment, the compound is

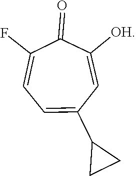 [SMILES: O=c1c(O)cc(C2CC2)ccc1F]

In an embodiment, the compound is

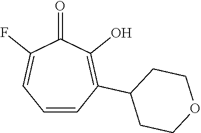 [SMILES: O=c1c(F)cccc(C2CCOCC2)c1O]

In an embodiment, the compound is

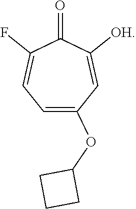 [SMILES: O=c1c(O)cc(OC2CCC2)ccc1F]

In an embodiment, the compound is:

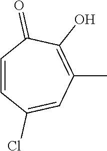

In an embodiment, the compound is:

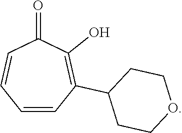 [SMILES: O=c1ccccc(C2CCOCC2)c1O]

In an embodiment, the compound is:

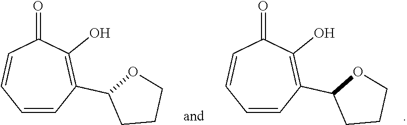

In an embodiment, the compound is:

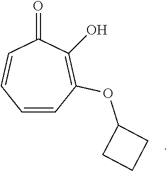 [SMILES: O=c1ccccc(OC2CCC2)c1O]

In an embodiment, the compound is:

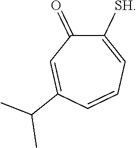

In an embodiment, the compound is:

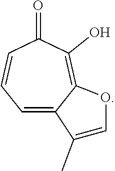

### Treatment of Neurodegenerative Diseases

Aceruloplasminemia is a rare autosomal recessive disease that is caused by a mutation in the gene encoding for ceruloplasmin (Cp) resulting in an absent or inactive multicopper oxidase glycoprotein. Without this protein iron (II) is unable to be oxidized to iron (III) and be available to bind to transferrin. This ultimately results in a decrease in holo-transferrin which is responsible for circulating iron across the body and has shown increased intracellular iron. Our EPR results show that Hinokitiol and FeM-1269 can mimic ferroxidase activity by promoting Fe(II) oxidation to Fe(III). Furthermore, we have also shown that Hinokitiol can handoff iron to transferrin allowing for the system to maintain homeostasis. We have also shown that hinokitiol-bound Fe(III) demonstrates mitigated capacity for neurodegeneration-promoting Fenton chemistry. These observations collectively support the use of Hino, FeM-1269, and other similarly acting tropolones as molecular prosthetics for ceruloplasmin to restore ferroxidase activity leading to recovery of systemic iron via increase in holo-transferrin levels as well as increased iron (III). In certain embodiments, iron and holo-transferrin concentrations in the brain before and after treatment with Hinokitiol may be tested in ceruloplasmin knock-out mice (CpKO). In further embodiments, iron (III) concentrations in blood and plasma samples from ceruloplasmin knock-out mice (CpKO) may be measured with ICP-MS with EPR.

In certain embodiments, holo-transferrin production may be measured using gel shift assays that can separate apo-transferrin from holo-transferrin indicating iron is being loaded onto the protein. In certain embodiments, recovery of iron (III) concentrations may be measured with cellular assays with ceruloplasmin knock-outs, iron specific binding dyes, and/or ICP-MS paired with EPR. In further embodiments, oxidative stress in the cells may be monitored via QT-PCR with ROS specific primers.

Treatment of Neurodegenerative Disease includes administration of any one of the compounds disclosed herein to a mammal in need thereof. One of skill in the art would administer said compounds by a method consistent with the mammal's medical history. The disease or condition being treated includes, but is not limited to, an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, an Idiopathic NBIA, a vascular dementia, a tauopathies, a progressive supranuclear palsy, a corticobasal degeneration, a subacute sclerosing panencephalitic parkinsonism, a postencephalitic parkinsonism, a guam parkinsonism-dementia complex, a Pick's disease, and a frontotemporal dementia.

### EXAMPLES

The present disclosure now being generally described will be more readily understood by reference to the following, which is included merely for purposes of illustration of certain aspects and embodiments of the present disclosure, and is not intended to limit the present disclosure.

### Example 1—Effect of Hinokitiol on Dopamine Oxidation

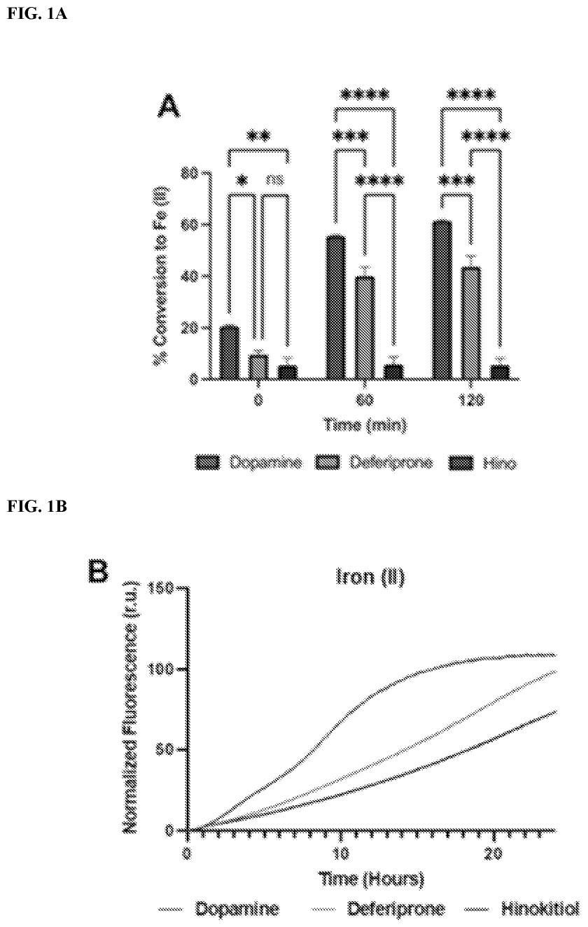

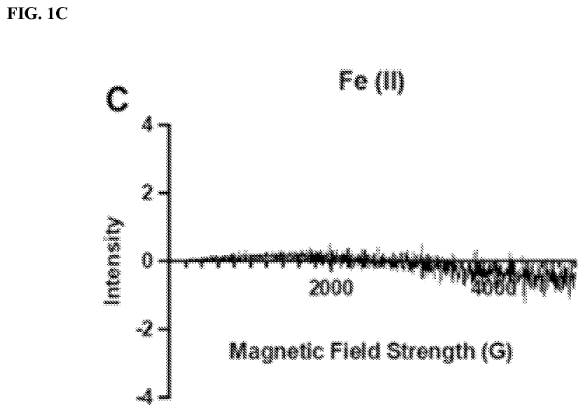

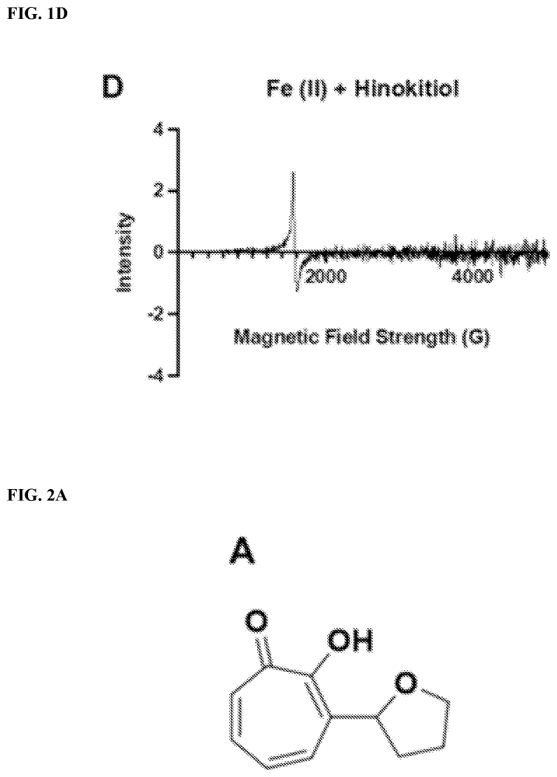

To measure the capacity of hinokitiol as a ‘safe passage’ for iron mobilization from neurons, the reactivity of Fe(III) complexed with dopamine, DFP, and hinokitiol using Ferrozine assay was measured and described (33). Iron (III) was precomplexed to either dopamine, hinokitiol, or DFP in HEPES buffer then added to a 96 well plate containing ferrozine and reducing agents and monitored via absorbance at 562 nm using a plate reader. We observed robust conversion to Fe(II) under reducing conditions when Fe(III) was complexed with dopamine and DFP, while complexation with hinokitiol significantly reduced the Fe(III) conversion (FIG. 1A). In a similar manner, using a ROS probe 2,7-dichlorofluorescin (DCF) showed that ROS production induced by Fe(II) was greatly reduced in the presence of hinokitiol compared to dopamine and DFP (FIG. 1B). The capacity to autoxidize Fe(II) by detecting the iron species resulting from the binding of hinokitiol and Fe(II) using electron paramagnetic resonance (EPR). Was observed at a signal at g′=4.3 when Fe(II) was bound by hinokitiol (FIG. 1D), suggesting the presence of high-spin Fe(III)-Hino complex in octahedral geometry. This peak was not observed when only Fe(II) was present (FIG. 1C), which suggests that Fe(II) was not spontaneously oxidized during measurement. In vitro data show that hinokitiol is better than DFP at maintaining iron as Fe³⁺. To conduct this assay, solutions of iron (II) was prepared in degassed 1:1 MeOH:H₂O with reducing agents, then after the Fe(II) spectra was obtained at 77K then the solution was allowed to warm to room temperature and hinokitiol was added in a 3:1 concentration. The spectra was obtained in which the g′=4.3 value was observed.

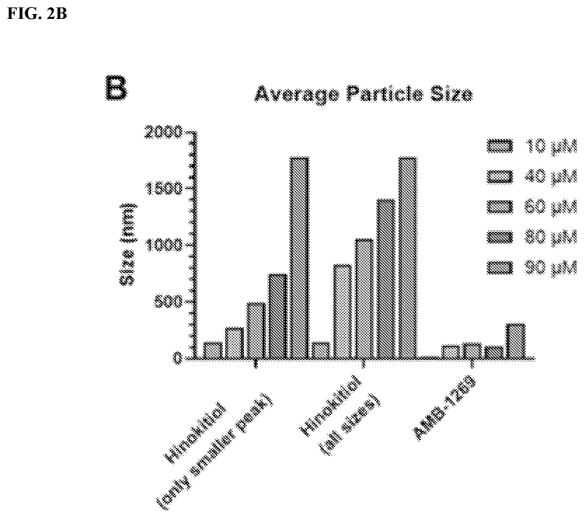

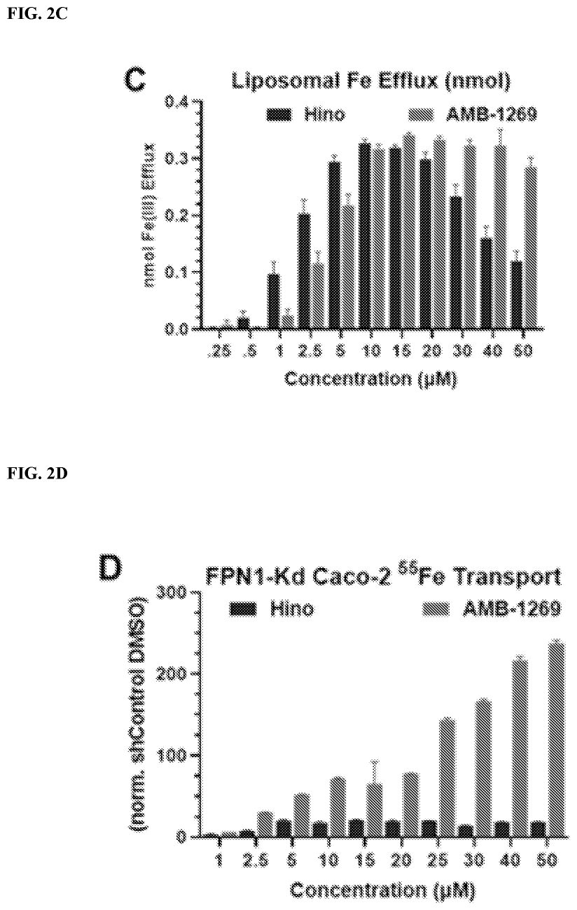

FeM-1269, a more effective and less toxic derivative (FIG. 2A), aggregated less than hinokitiol (FIG. 2B) and was able to transport iron in a wider concentration range compared to hinokitiol in liposomes (FIG. 2C) and Caco-2 cells (FIG. 2D).

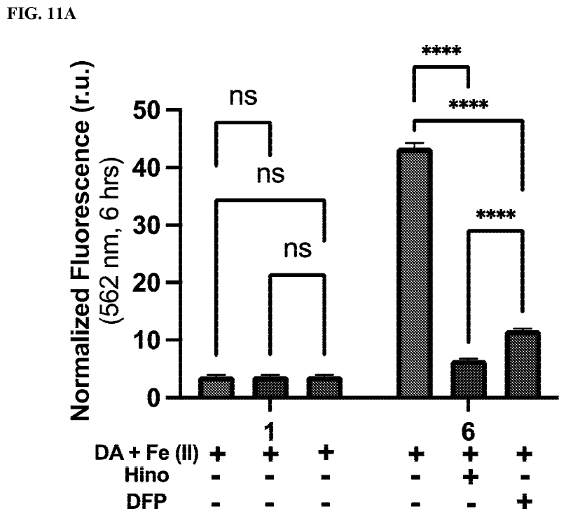

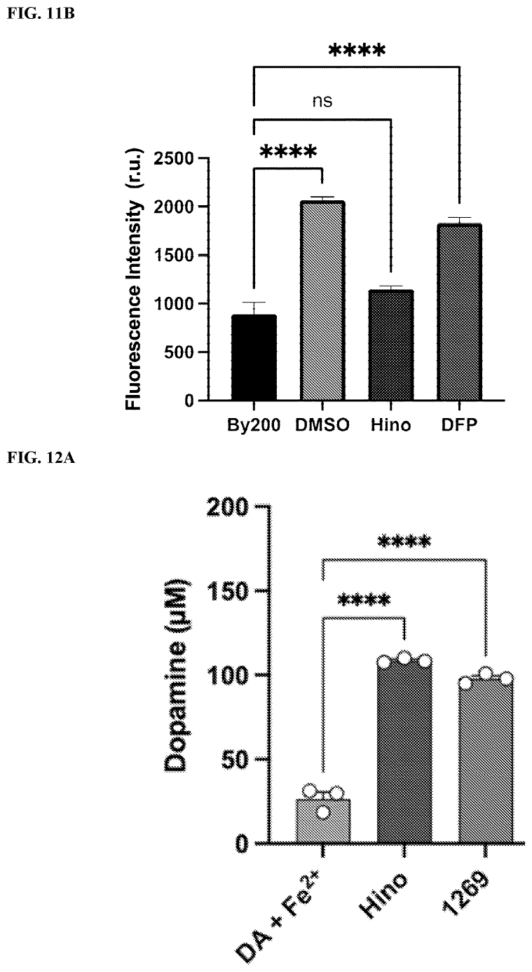

To probe the oxidation of dopamine and ROS production a fluorescent probe 2,7-dichlorofluorescin (DCF) was used in which dopamine was precomplexed with Fe(II) and monitored via plate reader for one hour then small molecules such as Hino or DFP were added and we see evidence of Hinokitiol mitigating the dopamine oxidation (FIG. 11A). To further probe this, FPN1.2KO C. elegans were plated on NGM plates seeded with OP50 at the L4 life stage then allowed to grow till day four and treated with small molecule hino or DFP then collected on day 5 and homogenized. The lysate was used in a 96 well plate with 2,7-dichlorofluorescin (DCF) to detect ROS. Hinokitiol could restore wild type levels of ROS in vivo (FIG. 11B).

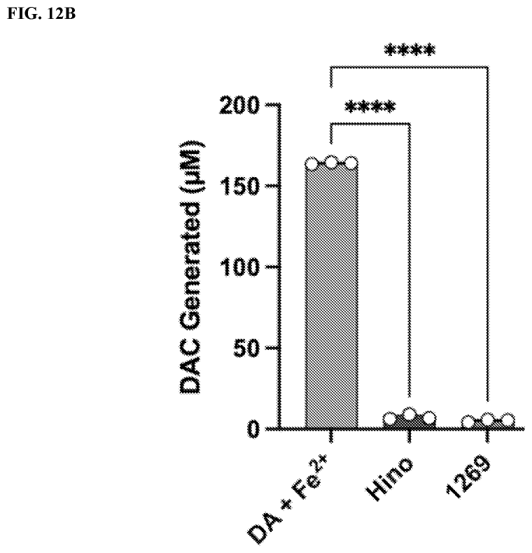

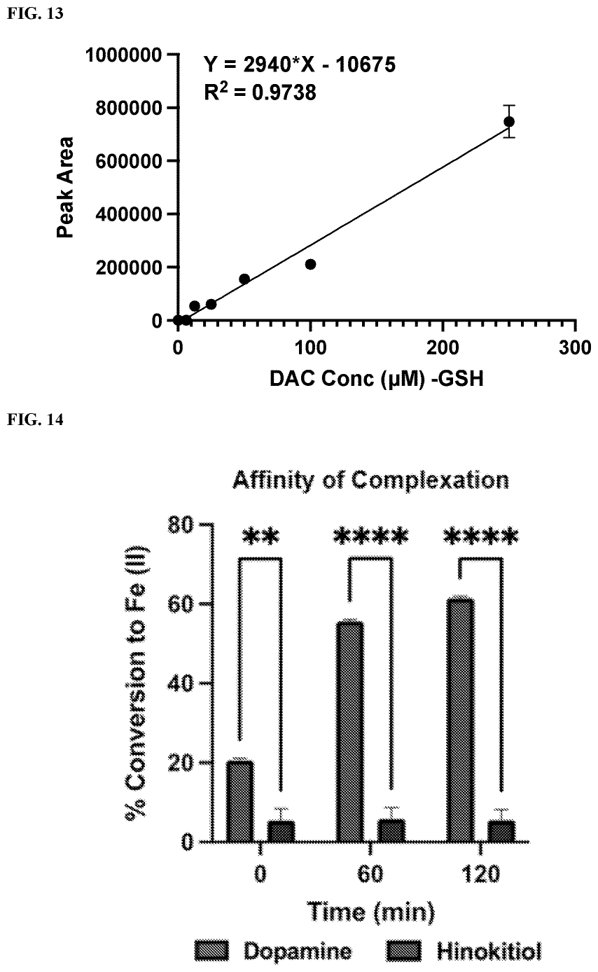

To monitor dopamine oxidation, an in vitro assay using an LC-MS was developed by monitoring dopamine concentrations via mass spectrometry hits. Samples of 100 μM dopamine were prepared and then treated with either control, hino, or FeM-1269. Both hino and FeM-1269 had evidence of mitigating dopamine oxidation (FIG. 12A). In addition, this was monitored via LC-MS to see the concentration of DAC generated in this same process. Similarly, hino and FeM-1269 reduced the quantity of dopamine being oxidized to form DAC (FIG. 12B). To determine linearity for the quantification of this assay, peak intensity was plotted against the increased concentration of DAC in which an R₂ value is 0.9738.

Finally, to determine the affinity of complexation the small molecules were precomplexed with iron (III) and then introduced in a plate reader with strong reducing agents and ferrozine dye to determine affinity of the complex. Hino has a strong affinity to bind to Fe(III) whereas the control of dopamine does not bind strongly to Fe(II) (FIG. 14)

### Example 2—Effect of Hinokitiol in C. elegans Neurodegeneration Model

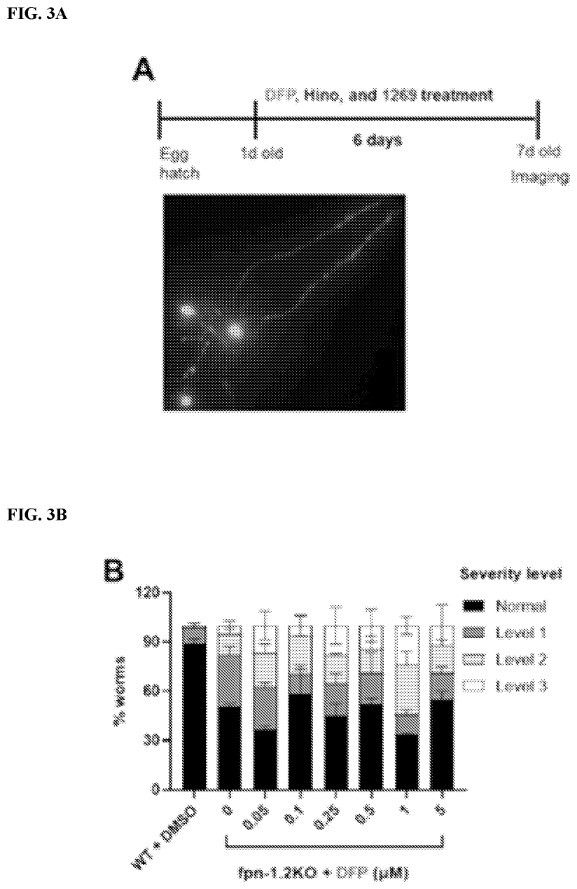

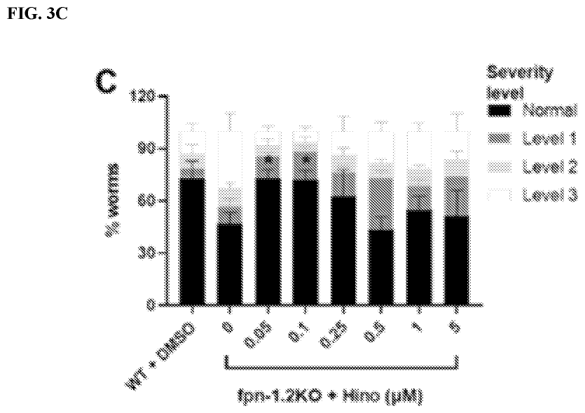

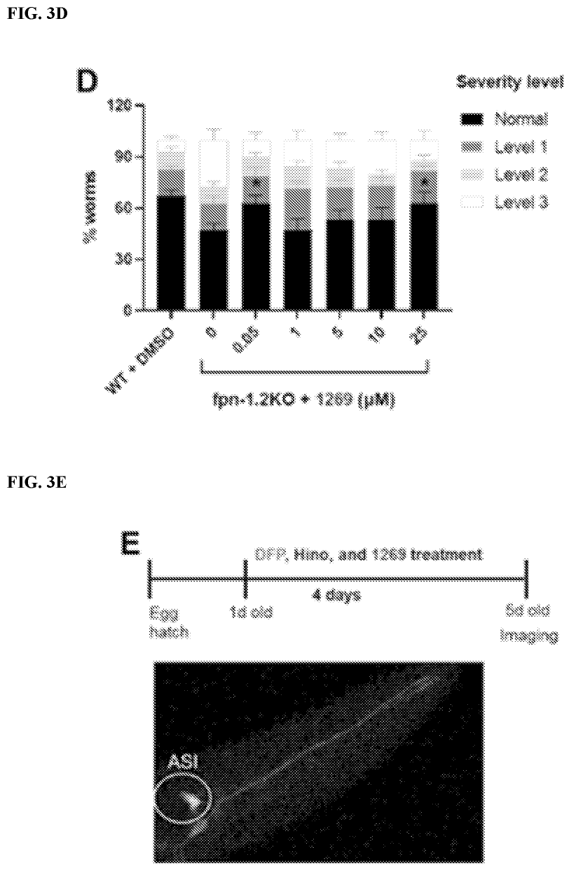

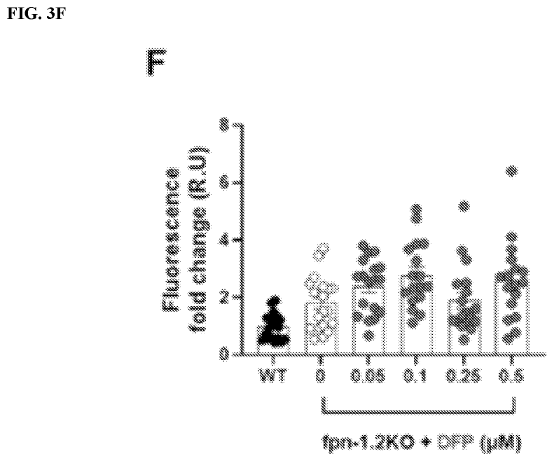

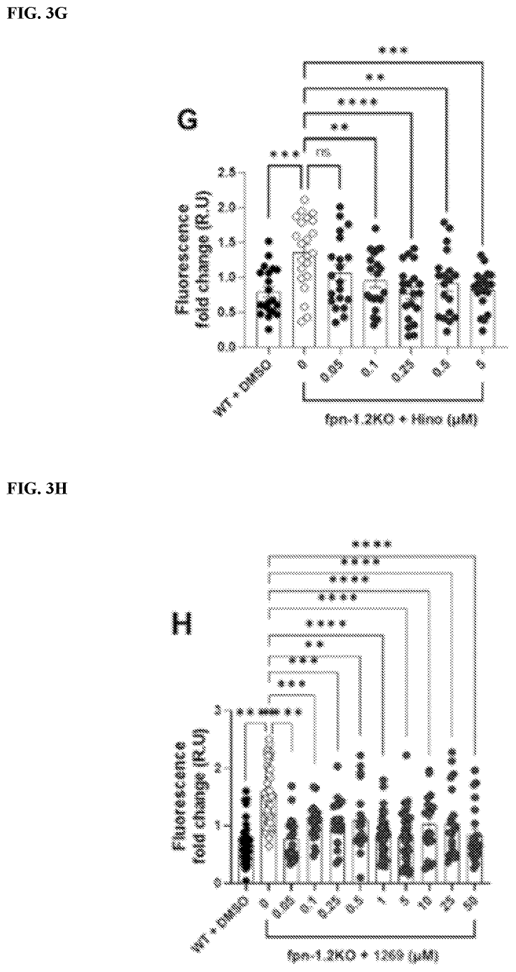

The ability of hinokitiol and FeM-1269 to confer neuroprotection. Replicated NBIA, was created fpn-1.2KO C. elegans strain using CRISPR/Cas9-mediated knockout and then mated with Pdat::GFP strain to create a fpn-1.2KO strain with GFP-tagged dopaminergic neurons (fpn-1.2KO; Pdat::GFP). The genotype of strains was verified by genotyping PCR and Sanger sequencing. All strains used have been backcrossed to N2 at least three times. To treat C. elegans with small molecules, day 4 adult worms were transferred to NGM containing small molecules. Worms were transferred to newly seeded small molecules containing NGM until the day seven in which they are imaged using a confocal microscope with GFP fluorescence. The scoring is conducted in a blinded study in which the integrity of the dopaminergic neuron is observed to reflect neurodegeneration. This mutant strain displayed dopaminergic neurodegeneration (FIGS. 3B-D) and elevated neuronal ferritin levels (FIGS. 3 F-H), which is a readout of intracellular iron levels. Using a specific treatment scheme (FIG. 3A) and neurodegeneration scoring method as reported (35, 36), it was found that DFP did not confer neuroprotective effects at all concentrations administered (FIG. 3B), while hinokitiol showed efficacy at low concentrations (0.05-0.1 μM) (FIG. 3C). Interestingly, FeM-1269 showed potency at concentration as low as 0.05 μM and the highest concentration (25 μM) (FIG. 3D). These results demonstrate the potential for hinokitiol and FeM-1269, and more broadly iron-transporting tropolones, to treat neurodegenerative disorders. Hinokitiol derivative FeM-1269 may have potential to increase the therapeutic index of hinokitiol. Further, in order to connect the neuroprotective effect with iron mobilizing capacity, a fpn-1.2KO strain was created with GFP-tagged ferritin (ftn-1) to measure iron levels in the representative ASI neurons (FIG. 3E). Consistent with FIG. 1B, DFP failed to reduce the fluorescence level at all administered concentrations (FIG. 3F), suggesting that DFP did not mobilize iron from fpn-1.2KO ASI neurons. On the other hand, hinokitiol and FeM-1269 treatment to fpn-1.2KO worms caused robust reductions of fluorescence levels (FIG. 3G-H) from the concentration of 0.1 μM and 0.05 μM, respectively, which can be associated with the release of intracellular iron from ASI neurons. These sets of data further support the conclusion that hinokitiol and FeM-1269 have the capacity to protect neurons from iron-induced neurodegeneration.

### Example 3—Effect of Hinokitiol in Flatiron Mouse Neurodegeneration Model

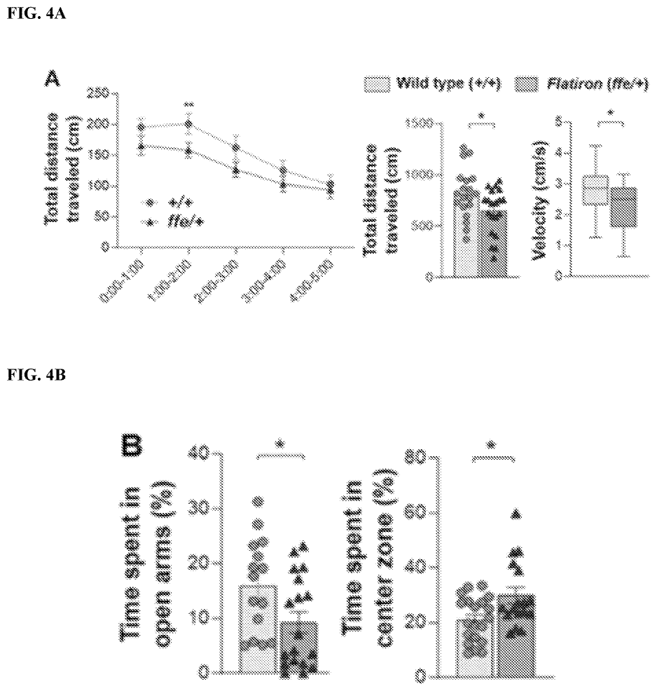

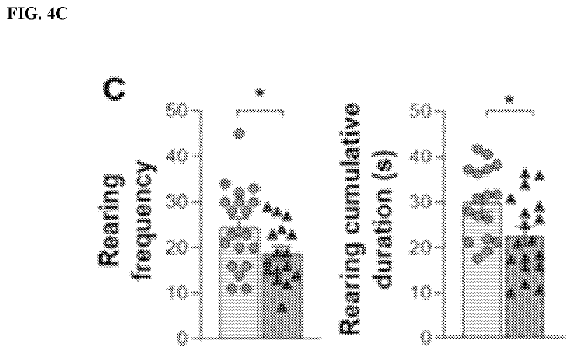

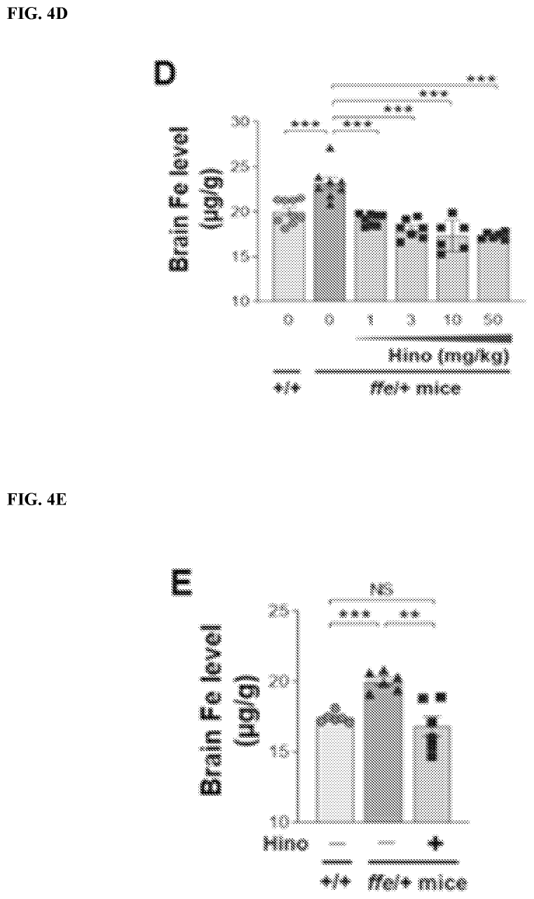

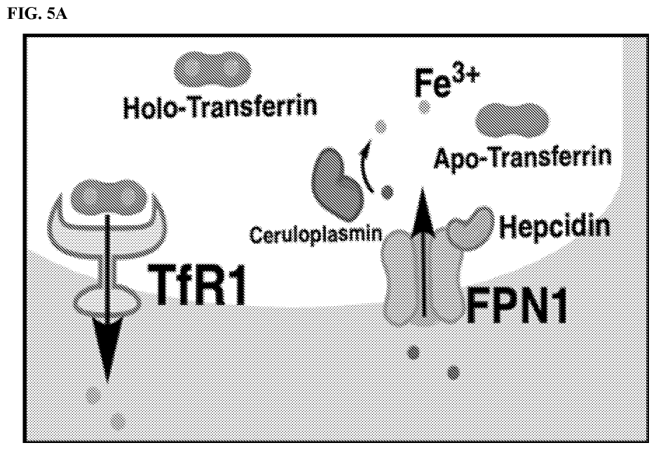

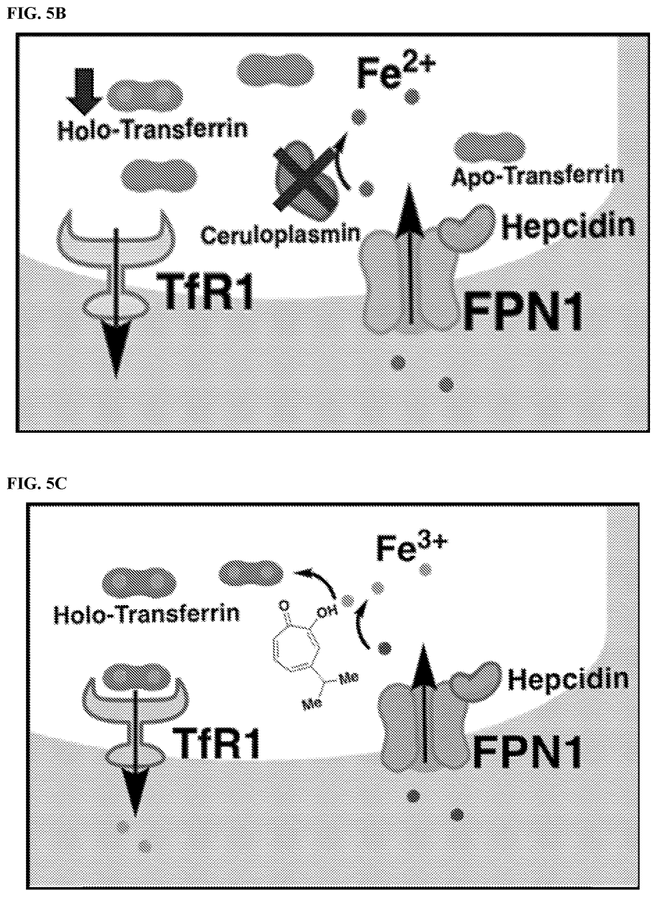

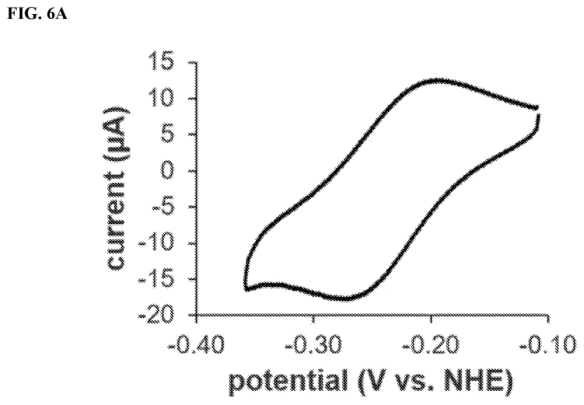

In parallel to C. elegans, flatiron mice were used, a leading animal model of FPN1 deficiency due to H32R mutation of FPN1 (34, 35). These mice were subjected to ICP-MS and behavioral studies in which blood samples and tissues were collected and submitted for analysis. In addition, behavior was assessed by an elevated plus maze, exploratory mazes, and daily monitoring. Due to iron accumulation in the brain (FIG. 3D-E), these mice displayed elevated anxiety levels (FIG. 4A-C). To determine dose-dependency these mice were subjected to a singular IP injection to assess acute administration and then treated chronically via IP injections of 10 mg/kg for a week. Acute administration of hinokitiol caused dose-dependent decrease of brain iron level (FIG. 4D). Similar effect was observed after chronic administration of 10 mg/kg hinokitiol after 7 days (FIG. 4E). Altogether, these data sets suggest that hinokitiol has the capacity to penetrate blood-brain barrier to mobilize accumulated brain iron in flatiron mice.

### Example 4—Cyclic Voltammetry of Hinokitiol-Iron Complexes

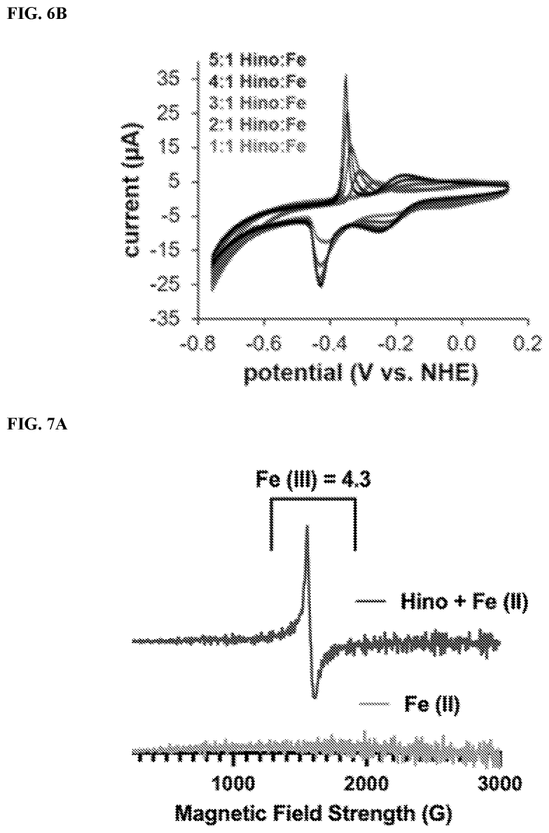

The redox potentials decreased with increasing hinokitiol concentrations. These were obtained with a 100 mV/s scan rate with a Hg electrode, Ag/AgCl reference, and graphite auxiliary using a 0.1 M Tris buffer in 1:1 MeOH:H₂O at pH=7.2 and 100 μM Fe(NO₃)₃. Data for these experiments are shown in FIG. 6A-FIG. 6B.

### Example 5—EPR of Hinokitiol-Iron Complexes

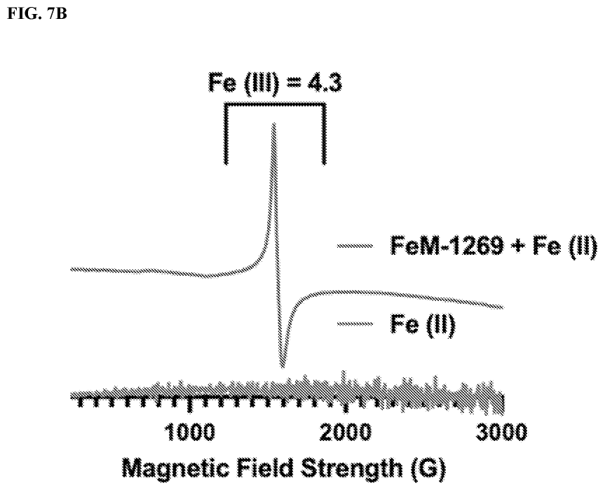

To conduct this assay, solutions of iron (II) was prepared in degassed 1:1 MeOH:H₂O with reducing agents, then after the Fe(II) spectra was obtained at 77K then the solution was allowed to warm to room temperature and hinokitiol was added in a 3:1 concentration. The spectra was obtained in which the g′=4.3 value was observed. Data for these experiments are shown in FIG. 7A-FIG. 7B.

### Example 6—Transferrin-Hinokitiol Iron Transfer Kinetics

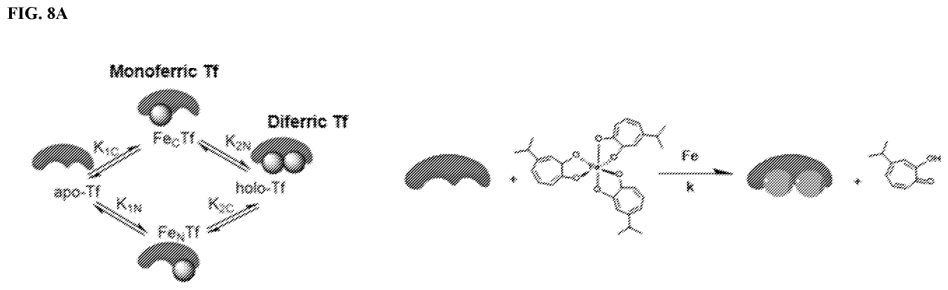

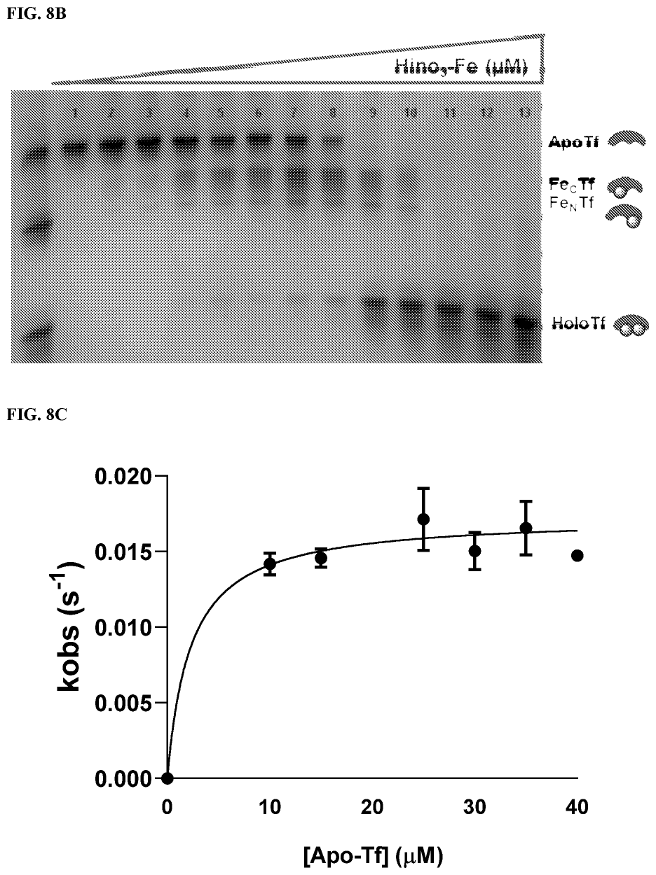

For kinetic experiments, 4 μM human apo-Tf was incubated with 64 μM Fe(Hino)₃ complex. The reactions were stopped at several time points using sample buffer. Samples were run using the same conditions as above. After running, the gels were stained with RAPIDStain (G-biosciences 786-31), according to the manufacturer's instructions. The formation of different Fe-Tf species was quantified by measuring the band densities using ImageLab 4.1 (Bio-Rad) and expressed as a function of molar fractions. Data for these experiments are shown in FIG. 8B-FIG. 8C.

### Example 7—Colorimetric/Fluorometric Iron (II) Conversion Assays

For the colorimetric assay: Small molecules were precomplexed to Fe(III) in a 3:1 ratio and then placed in a 96 well plate compatible with a plate reader. Then a solution containing HEPES buffer, ferrozine dye, and a reducing agent was added to the wells and absorbance at 562 nm was reported for multiple hours.

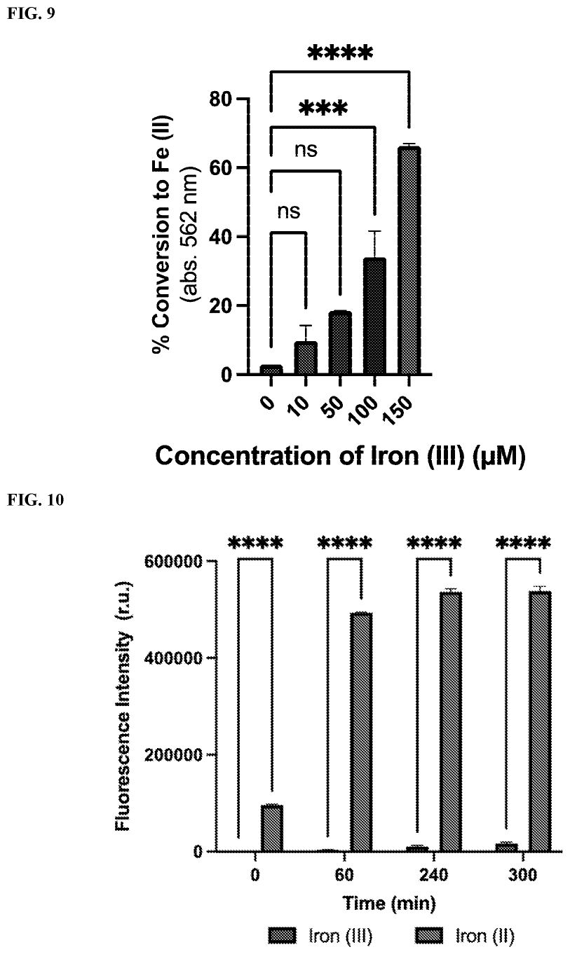

For the fluorometric assay: Solutions of Iron (II) and Iron (III) were freshly prepared and placed into a 96 well plate compatible with a plate reader and then an ROS dye 2,7-dichlorofluorescin (DCF) was added to the plate and fluorescence monitoring at 488 nm was observed. Data for these experiments are shown in FIG. 9 & FIG. 10.

### Example 8—Synthesis and Characterization of Compounds of the Disclosure

The compounds of the present disclosure have been synthesized and characterized previously. See, e.g., WO 2021/076938 (PCT/US2020/056048), WO 2021/076945 (PCT/US2020/056056), and US 2021/0163393 (U.S. Ser. No. 17/046,608), the contents of each of which are expressly incorporated by reference herein.

### Example 9—Ligand Facilitated Fe(III) Efflux from Liposomes

Preparation of POPC (1-palmitoyl-2-oleoyl-sn-glycero-3-phosphocholine):Cholesterol Liposomes:

A 1M buffer solution of MES and Tris was prepared by dissolving 121.14 grams of Tris base and 213.25 grams of MES hydrate in 500 mL of MilliQ water and adjusted to pH7.0 using an 18M HCl solution before bringing the total volume of the solution to 1 L. A 500 mM solution of FeCl₃ was prepared by dissolving 0.811 grams of anhydrous FeCl₃ in 10 mL of a 0.1M H₂SO₄ aqueous solution. Inside buffer is prepared in a 50 mL falcon tube, which was added with 25 mL of MilliQ water, 1.61 g of sodium citrate, 1.5 mL of the above FeCl₃ solution, and 2.5 mL of the 1M MES/Tris HCl buffer (pH=7.0), and finally additional MilliQ H₂O to bring to a 50 mL final volume. The inside buffer prepared will be a solution with final concentrations of 15 mM of FeCl₃, 125 mM of sodium citrate, and 50 mM of MES/Tris HCl at pH7.0.

Lipid solution is prepared by dissolving 206.9 mg of POPC (1-palmitoyl-2-oleoyl-sn-glycero-3-phosphocholine) and 8.6 mg of cholesterol in 10 mL of ethanol.

The lipids solution and the inside buffer solution are independently loaded into 10 mL luer lock syringes for loading into a virgin cartridge on a Precision Nanosystems NanoAssembler to prepare unilamellar liposomes with the following parameters: 7.5 mL total volume, 1.5:1 mixing ratio of inside buffer:lipids solution, 8 mL/min flow rate, ambient temperature, 0.35 mL start waste, and 0.05 mL of end waste. 7.5 mL of liposomes is harvested for purification on a 6-inch long, 1-inch diameter Sephadex G-50 column wetted in 600 mM sodium ascorbate and 50 mM MES/Tris HCl pH 7.0 buffer. This buffer also serves as the column running buffer. The crude liposome solution is carefully loaded with minimal volume of running buffer above the top of the Sephadex, allowed to enter the matrix, and the column is run with the continued addition of running buffer. The eluting liposomes, observed as a milky and turbid solution, are collected until free iron begins to elute (observed as a deep purple color) and the fractions are pooled for phosphorus quantitation.

### Determination of Phosphorus Content:

Phosphorus content of eluted liposomes was determined by the process outlined below. 10 μL of liposome elution and a running buffer blank are added to a 5 mL glass vial containing 450 μL of a 8.9 M aqueous H₂SO₄ solution, the mixture is heated to 225° C. for 25 minutes in an aluminum heat block to hydrolyze POPC and cooled for 5 minutes. 200 μL of a 30% hydrogen peroxide aqueous solution is added to each vial and heated to 225° C. for 25 minutes. After cooling, the phosphorus content was determined using an Abcam Phosphate Assay kit, with buffer subtracted phosphorus levels determined against a standard curve of phosphate included in the kit. Liposomes were diluted to 1 mM phosphate in Running Buffer for use in assays after accounting for dilutions made during phospholipid digestion.

### Determination of the Iron Transporting Rate Constant of the Ligands:

Determination of the rate at which a small molecule ligand liberates (transport) Ferric iron from liposomes is performed in clear bottom black 384-well plates on a Spectramax i3x set to read the absorbance at 562 nm in the kinetic mode every 60 seconds for 120 minutes. 1 μL of serially diluted DMSO stock solution of a small molecule ligand is added to wells in triplicate to give the final concentrations of 40, 20, 10.5, 2.5, and 1.25 UM of the ligand at 80 μL final volume, followed by addition of 1 μL of 100 mM Ferrozine in water to give a final concentration of 1 mM ferrozine. 78 μL of liposomes diluted to 1 mM phosphorus in running buffer is added to wells as quickly as possible (using a digital repeater multichannel pipette) with the kinetic read initiated as rapidly as possible after addition of liposomes to all wells. Typically, eight compounds at six concentrations are tested in triplicate simultaneously.

Upon completion of kinetic read, the data for individual reads is fitted to a single phase association regression with the equation Y═(Y₀−Y_{Max})^{(-kX)}+Y_{Max}, with Y being the 562 absorbance value and X being time in minutes. The k value from the individual replicate values is averaged from the triplicates runs for each compound concentration. The ligand's ability in liberating iron from within liposome is represented by the rate, k, at a given concentration. Rate k is considered as the efflux rate and ligands are ranked by the efflux rate at 10 M ligand concentration at which they effect Ferric iron efflux.

### Example 10—shDMT1-Caco2 ⁵⁵Fe Transport Assay to Assess Ligand Ability in Transporting Fe (III)

### Materials and Methods:

Cells: DMT1-deficient Caco-2 cells (alias: “shDMT1,” or “4A” cells) were from Grillo et al. Science, 2017, and were cryopreserved in liquid nitrogen prior to use. Reagents and supplies: 55FeCl₃ was obtained from PerkinElmer (Boston, MA). Iron (III) chloride (FeCl₃) hexahydrate was obtained from Sigma. Dulbecco's Modified Eagle Medium (DMEM), fetal bovine serum (FBS), L-glutamine, MEM nonessential amino acids, penicillin-streptomycin, G418, formic acid, methanol, high-purity water, ammonium formate, dimethyl sulfoxide (DMSO) were purchased from Fisher Scientific. Propranolol, atenolol and carbutamide were obtained from Sigma-Aldrich Chemical Company (St Louis, MO). Scintillation cocktail was obtained from Research Product International Co. (Mount Prospect, IL). Stericup filter system (PES membrane, 0.22 μm pore size) was purchased from Fisher Scientific. Corning item #3378 24-well transwell insert plates.

Test articles: known compounds hinokitiol and deferiprone were purchased from Sigma. They are tested side by side with the small molecule ligands disclosed in this application. DMSO stocks (10 mM, which is 1,000x of the 10 UM dose level) of hinokitiol or test articles were prepared. A stock solution of 25 mM deferiprone in DMSO was prepared. The DMSO stock solutions were stored at −20° C. or below when not in use.

The growth medium was prepared according to the following table:

1L

| | | | | For 1L |
|---|---|---|---|---|
| Component | Stock | Final | | (mL) |
| DMEM | --- | - | | 1,000 |
| FBS | --- | 10% | total | 116.2 |
| Glutamine | 200 mM | 4 | mM (2%) | 23.2 |
| PEN-STREP | 1000 μg/mL | 100 | μg/mL (1%) | 11.6 |
| N.E. Amino Acids | --- | 1% | total | 11.6 |
| Geneticin G418 disulfate | | 800 | mg/L | |

Apical media (serum free DMEM, 10 mM MES, pH 6.5) was prepared. Apical master mix media was prepared fresh with the addition of 200 nM ⁵⁵Fe before each experiment. For the negative control propranolol and atenolol wells, 200 nM of non-radioactive iron was used.

The basolateral media was serum-free DMEM, 10 mM HEPES, 2% bovine serum albumin (BSA), pH 7.4.

For each experiment, cells were seeded into the 24-well transwell plates (0.5 mL of 50,000 cells/mL) with growth media. The basolateral companion plate was loaded with 1 mL of growth media. After 12-24 hr, both the apical and basolateral chamber were replaced with growth media. The apical media was changed 3× per week for 21-28 days, including a media change exactly 48 hr before the assay date.

On the assay day, the TEER values were measured and the average TEER value was obtained. Individual wells with TEER value >35% lower than the average of all wells were excluded. For the qualified wells, the apical layer (twice) and basolateral (once) chambers were washed with PBS. The basolateral companion plate was then filled with 1 ml of basolateral media. Via addition down the side-wall of the apical well, 300 μL per well of the apical assay master mix, with the indicated dose level of test article, was added. Each dose was tested in triplicate. The plates were incubated (5% CO₂ and 90% humidity at 37° C.) for the indicated timepoints. At each indicated timepoint, the basolateral supernatant was gently mixed via pipetting, and 200 μL of the basolateral supernatant was transferred to scintillation vials. To each scintillation counting vial, 5 mL of scintillation cocktail fluid was added. For each scintillation vial, the radioactivity (CPM) was determined with liquid scintillation counter LS6500. The counting time per vial was 5 min.

### Data Processing:

All raw CPM values are divided by the average value of blank DMSO solution to give a fold of change above the DMSO value. The mean and standard deviation of each compound at each concentration level and at each time point measured was calculated to give the fold change (fc) value. For rank order compounds, the fc value of a ligand at 4 h time point is further divided by the fold change (fc) value of hinokitiol (a positive control compound) at an equimolar concentration to arrive at the FC (normalized fold of change) value.

### REFERENCES CITED

1. R. J. Ward, F. A. Zucca, J. H. Duyn, R. R. Crichton, L. Zecca, The role of iron in brain ageing and neurodegenerative disorders. Lancet Neurol 13, 1045-1060 (2014).
2. B. B. Muhoberac, R. Vidal, Abnormal iron homeostasis and neurodegeneration. Frontiers in aging neuroscience 5, 32 (2013).
3. R. M. Uranga, G. A. Salvador, Unraveling the Burden of Iron in Neurodegeneration: Intersections with Amyloid Beta Peptide Pathology. Oxid Med Cell Longev 2018, 2850341 (2018).
4. L. Balejcikova, K. Siposova, P. Kopcansky, I. Safarik, Fe(II) formation after interaction of the amyloid beta-peptide with iron-storage protein ferritin. J Biol Phys 44, 237-243 (2018).
5. J. S. Cristovao, R. Santos, C. M. Gomes, Metals and Neuronal Metal Binding Proteins Implicated in Alzheimer's Disease. Oxid Med Cell Longev 2016, U.S. Pat. No. 9,812,178 (2016).
6. C. Cheignon et al., Oxidative stress and the amyloid beta peptide in Alzheimer's disease. Redox Biol 14, 450-464 (2018).
7. H. Jiang, J. Wang, J. Rogers, J. Xie, Brain Iron Metabolism Dysfunction in Parkinson's Disease. Mol Neurobiol 54, 3078-3101 (2017).
8. A. P. Lan, J. Chen, Z. F. Chai, Y. Hu, The neurotoxicity of iron, copper and cobalt in Parkinson's disease through ROS-mediated mechanisms. Biometals: an international journal on the role of metal ions in biology, biochemistry, and medicine 29, 665-678 (2016).
9. S. Chiang, D. S. Kalinowski, P. J. Jansson, D. R. Richardson, M. L. Huang, Mitochondrial dysfunction in the neuro-degenerative and cardio-degenerative disease, Friedreich's ataxia. Neurochemistry international 117, 35-48 (2018).
10. F. Lupoli, T. Vannocci, G. Longo, N. Niccolai, A. Pastore, The role of oxidative stress in Friedreich's ataxia. FEBS letters 592, 718-727 (2018).
11. M. Muller, B. R. Leavitt, Iron dysregulation in Huntington's disease. Journal of neurochemistry 130, 328-350 (2014).
12. S. Agrawal, J. Fox, B. Thyagarajan, J. H. Fox, Brain mitochondrial iron accumulates in Huntington's disease, mediates mitochondrial dysfunction, and can be removed pharmacologically. Free radical biology & medicine 120, 317-329 (2018).
13. A. Gregory, S. Hayflick, Neurodegeneration with Brain Iron Accumulation Disorders Overview.
14. P. Hogarth, Neurodegeneration with brain iron accumulation: diagnosis and management. J Mov Disord 8, 1-13 (2015).
15. M. C. Kruer et al., Neuroimaging features of neurodegeneration with brain iron accumulation. AJNR Am J Neuroradiol 33, 407-414 (2012).
16. E. Madsen, J. D. Gitlin, Copper and iron disorders of the brain. Annu Rev Neurosci 30, 317-337 (2007).
17. B. Todorich, X. Zhang, B. Slagle-Webb, W. E. Seaman, J. R. Connor, Tim-2 is the receptor for H-ferritin on oligodendrocytes. Journal of neurochemistry 107, 1495-1505 (2008).
18. G. A. Salvador, Iron in neuronal function and dysfunction. Biofactors 36, 103-110 (2010).
19. S. J. Dixon, B. R. Stockwell, The role of iron and reactive oxygen species in cell death. Nat Chem Biol 10, 9-17 (2014).
20. G. A. Salvador, R. M. Uranga, N. M. Giusto, Iron and mechanisms of neurotoxicity. Int J Alzheimers Dis 2011, 720658 (2010).
21. H. M. Schipper, Brain iron deposition and the free radical-mitochondrial theory of ageing. Ageing Res Rev 3, 265-301 (2004).
22. R. C. Hider, Y. Ma, F. Molina-Holgado, A. Gaeta, S. Roy, Iron chelation as a potential therapy for neurodegenerative disease. Biochemical Society transactions 36, 1304-1308 (2008).
23. G. Abbruzzese et al., A pilot trial of deferiprone for neurodegeneration with brain iron accumulation. Haematologica 96, 1708-1711 (2011).
24. D. Devos et al., Targeting chelatable iron as a therapeutic modality in Parkinson's disease. Antioxid Redox Signal 21, 195-210 (2014).
25. N. Parakh et al., Neurological Complications and Cataract in a Child With Thalassemia Major Treated With Deferiprone. J Pediatr Hematol Oncol 37, 433-444 (2015).
26. N. Mobarra et al., A Review on Iron Chelators in Treatment of Iron Overload Syndromes. International Journal of Hematology-Oncology and Stem Cell Research 10, 239-247 (2016).
27. F. N. Al-Refaie, B. Wonke, A. V. Hoffbrand, Deferiprone-associated myelotoxicity. European journal of haematology 53, 298-301 (1994).
28. A. S. Grillo et al., Restored iron transport by a small molecule promotes absorption and hemoglobinization in animals. Science 356, 608-616 (2017).
29. M. T. Nunez, P. Chana-Cuevas, New Perspectives in Iron Chelation Therapy for the Treatment of Neurodegenerative Diseases. Pharmaceuticals (Basel) 11 (2018).
30. J. L. Hamilton, M. I. Ul-Haq, A. L. Creagh, C. A. Haynes, J. N. Kizhakkedathu, Iron Binding and Iron Removal Efficiency of Desferrioxamine Based Polymeric Iron Chelators: Influence of Molecular Size and Chelator Density. Macromol Biosci 17 (2017).
31. G. Crisponi et al., A Speciation Study on the Perturbing Effects of Iron Chelators on the Homeostasis of Essential Metal Ions. PloS one 10, e0133050 (2015).
32. S. Ekaputri et al., A small molecule redistributes iron in ferroportin-deficient mice and patient-derived primary macrophages. Proceedings of the National Academy of Sciences 119, e2121400119 (2022).
33. Y. Sun, A. N. Pham, T. D. Waite, Elucidation of the interplay between Fe(II), Fe(III), and dopamine with relevance to iron solubilization and reactive oxygen species generation by catecholamines. J Neurochem 137, 955-968 (2016).
34. I. E. Zohn et al., The flatiron mutation in mouse ferroportin acts as a dominant negative to cause ferroportin disease. Blood 109, 4174-4180 (2007).
35. A. Donovan et al., The iron exporter ferroportin/Slc40a1 is essential for iron homeostasis. Cell metabolism 1, 191-200 (2005).

### INCORPORATION BY REFERENCE

All US patents and US and PCT patent application publications mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

What is claimed is:

1. A method of treating a disease or condition selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA, comprising administering to a subject in need thereof a therapeutically effective amount of a compound, or a pharmaceutically acceptable salt thereof, selected from the group consisting of hinokitiol, hinokitiol derivatives, and iron-transporting tropolones.

2. A method of treating a disease or condition selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA, comprising administering to a subject in need thereof a therapeutically effective amount of a compound, or a pharmaceutically acceptable salt thereof, having a structure according to Formula (Ia): 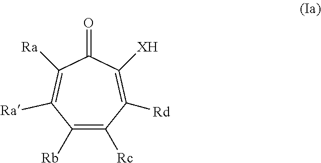
wherein:
X represents oxygen or sulfur;
Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
Rₐ′ represents hydrogen, halo, alkyl, or substituted alkyl; and R_{b}, R_{c}, and R_{d} are independently selected from the group consisting of hydrogen, halo, alkyl, substituted alkyl, heteroalkyl, alkylcycloalkyl, alkylheterocycloalkyl, alkoxy, substituted alkoxy, alkoxyalkyl, substituted alkoxyalkyl, cycloalkyloxy, substituted cycloalkyloxy, heterocycloalkyloxy, substituted heterocycloalkyloxy, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl; provided that Rₐ, R_{b}, R_{c}, and R_{d} are not all hydrogen.

3. The method of claim 2, wherein Rₐ′ is hydrogen.

4. The method of claim 2, wherein Rₐ′ is halo.

5. The method of claim 2, wherein Rₐ′ is alkyl, or substituted alkyl.

6. The method of any one of claims 2-5, wherein at least one of Rₐ, R_{b}, R_{c}, and R_{d} is selected from the group consisting of halo, alkyl, substituted alkyl, heteroalkyl, alkylcycloalkyl, alkylheterocycloalkyl, alkoxy, substituted alkoxy, alkoxyalkyl, substituted alkoxyalkyl, cycloalkyloxy, substituted cycloalkyloxy, heterocycloalkyloxy, substituted heterocycloalkyloxy, cycloalkyl, substituted cycloalkyl, heterocycloalkyl, and substituted heterocycloalkyl.

7. The method of any one of claims 2-6, wherein at least one of Rₐ, Rₐ′, R_{b}, R_{c}, and R_{d} is selected from the group consisting of methyl, ethyl, n-propyl, and isopropyl.

8. The method of claim 2 or 3, wherein the compound is represented by Formula Ib: 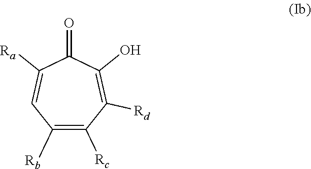
wherein:
Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl; and
R_{b}, R_{c}, and R_{d} are independently selected from the group consisting of hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, and heteroalkynyl;
provided that Rₐ, R_{b}, R_{c}, and R_{d} are not all hydrogen.

9. The method of any one of claims 2-8, wherein:
each occurrence of heterocycloalkyl is independently selected from the group consisting of: 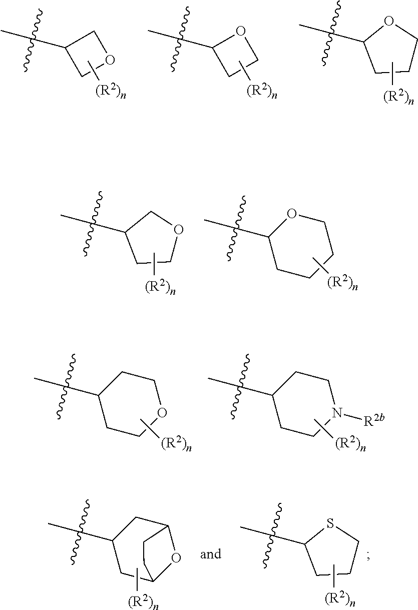
n is independently for each occurrence an integer selected from 0-5 inclusive;
R^{2b} is independently for each occurrence hydrogen, alkyl, substituted alkyl, heteroalkyl, hydroxy, alkoxy, substitutedalkoxy, alkoxyalkyl, substituted alkoxyalkyl, cycloalkyl, substituted cycloalkyl, alkenyl, substituted alkenyl, cycloalkenyl, substituted cycloalkenyl, alkynyl, and substituted alkynyl; and
each instance of R² independently represents —F, alkyl, haloalkyl, or alkoxy; or two geminal instances of R² represent carbonyl.

10. The method of any one of claims 2-9, wherein Rₐ represents —F, —CF₃, (C2-C15)alkyl, or substituted (C1-C15)alkyl.

11. The method of claim 10, wherein:
R_{b}, R_{c}, and R_{d} independently represent hydrogen, (C1-C15)alkyl, 3-12 membered cycloalkyl, 3-12 membered heterocycloalkyl, (C1-C15)alkylene-R₁, 3-12 membered cycloalkyl-R₁, or 3-12 membered heterocycloalkyl-R₁; and
R₁ represents independently for each occurrence halo, alkyl, alkoxy, or hydroxyl.

12. The method of claim 8, wherein at least one of Rₐ, R_{b}, R_{c}, and R_{d} is selected from the group consisting of methyl, ethyl, n-propyl, and isopropyl.

13. The method of any one of claims 11-12, wherein said compound is selected from the group consisting of:

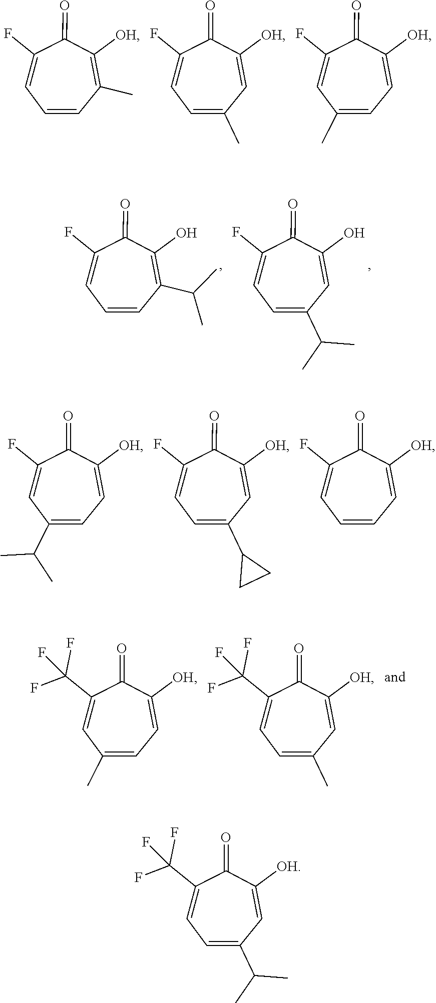

14. The method of claim 13, wherein said compound is

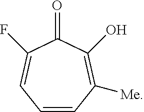 [SMILES: Cc1cccc(F)c(=O)c1O]

15. The method of claim 13, wherein said compound is

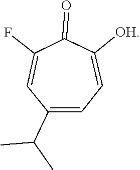

16. The method of claim 11, wherein said compound is selected from the group consisting

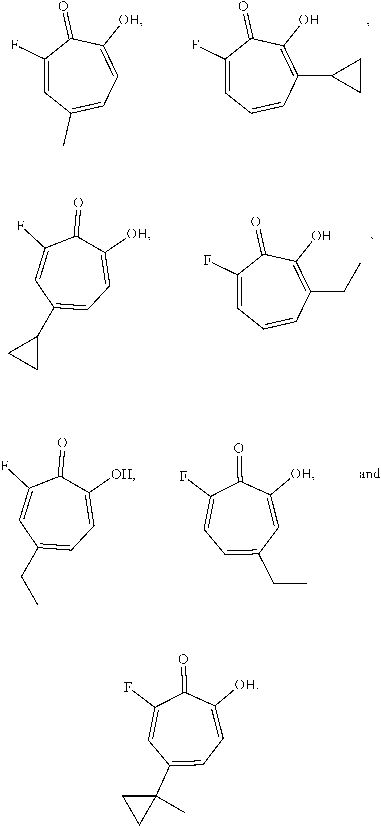

17. The method of claim 11, wherein said compound is

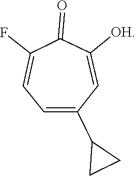 [SMILES: O=c1c(O)cc(C2CC2)ccc1F]

18. The method of any one of claims 2-9, wherein Rₐ represents —Cl, —F, —CF₃, (C2-C15)alkyl, or substituted (C1-C15)alkyl.

19. The method of any one of claim 18, wherein Rₐ represents —F.

20. The method of any one of claims 18-19, wherein:
R_{b}, R_{c}, and R_{d} independently represent hydrogen, halo (C1-C15)alkyl, 3-12 membered cycloalkyl, 3-12 membered heterocycloalkyl, (C1-C15)alkylene-R₁, 3-12 membered cycloalkyl-R₁, or 3-12 membered heterocycloalkyl-R₁; and
R₁ represents independently for each occurrence halo, alkyl, alkoxy, or hydroxyl.

21. The method of claim 20, wherein said compound is selected from the group consisting

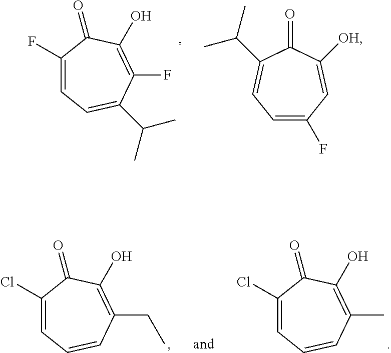

22. The method of any one of claims 2-9, wherein:
R_{b}, R_{c}, and R_{d} independently represent hydrogen or 3-12 membered heterocycloalkyl optionally substituted with one or two instances of R₂;
wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

23. The method of claim 22, wherein said compound is selected from the group consisting of:

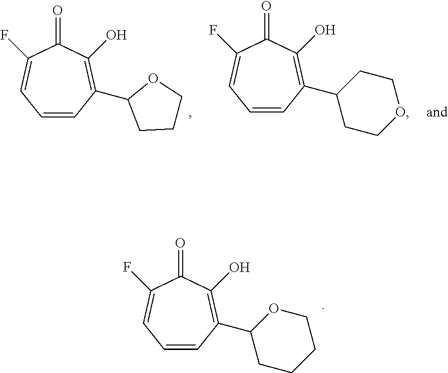 [SMILES: O=c1c(F)cccc(C2CCCO2)c1O ; O=c1c(F)cccc(C2CCOCC2)c1O ; O=c1c(F)cccc(C2CCCCO2)c1O]

24. The method of claim 23, wherein said compound is

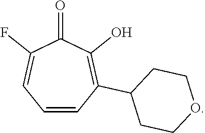 [SMILES: O=c1c(F)cccc(C2CCOCC2)c1O]

25. The method of claim 22, wherein:
said heterocycloalkyl comprises one or two nitrogen atoms; and at least one of said nitrogen atoms is represented by N(R_{2b});
R_{2b} independently represents hydrogen, —C(O)R₅, or —C(O)OR₅; and
R₅ independently represents hydrogen, alkyl, or substituted alkyl.

26. The method of any one of claims 2-9, wherein:
R_{b}, R_{c}, and R_{d} independently represent hydrogen or —OR₃; and
R₃ independently represents (C1-C15)alkyl, 3-12 cycloalkyl or bicycloalkyl, 3-12 membered heterocycloalkyl or heterobicylcloalkyl comprises one or two oxygen atoms, one or two nitrogen atoms, one or two sulfur atoms, or any combination of two atoms selected from the group consisting of oxygen, nitrogen, and sulfur atoms.

27. The method of claim 26, wherein said compound is:

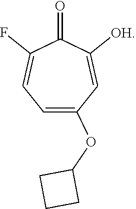 [SMILES: O=c1c(O)cc(OC2CCC2)ccc1F]

28. The method of claim 26, wherein said compound is selected from the group consisting of:

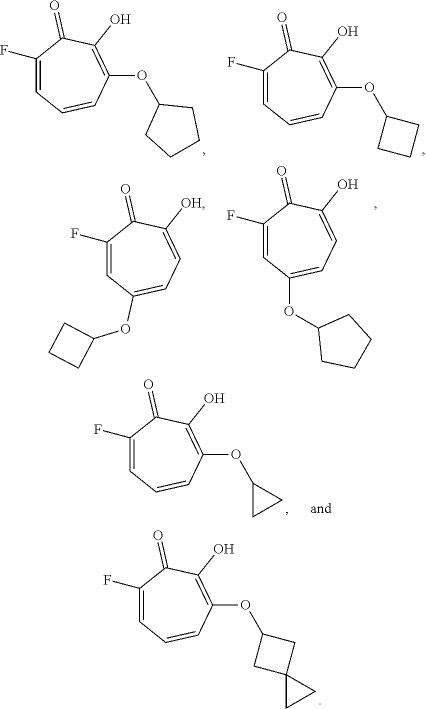

29. The method of claim 26, wherein each instance of R³ is optionally substituted with R₄; and R₄ independently represents alkyl, halogen-substituted alkyl, alkoxy, or hydroxy.

30. The method of any one of claims 2-10, wherein Rₐ represents —CH₃.

31. The method of claim 30, wherein:
R_{b}, R_{c}, and R_{d} independently represent hydrogen, (C1-C15) alkyl, 3-12 membered cycloalkyl, 3-12 membered heterocycloalkyl, (C1-C15) alkylene-R₁, 3-12 membered cycloalkyl-R₁, or 3-12 membered heterocycloalkyl-R₁; and
R₁ represents independently for each occurrence halo, alkyl, alkoxy, or hydroxy.

32. The method of claim 31, wherein said compound is selected from the group consisting of:

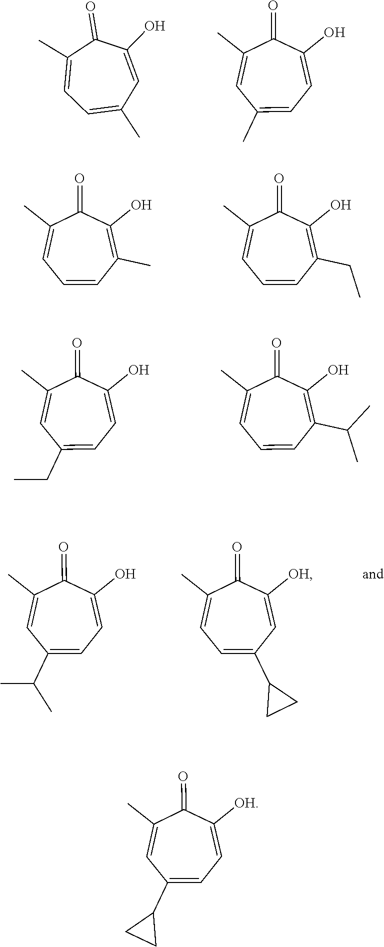

33. The method of claim 30, wherein:
R_{b}, R_{c}, and R_{d} independently represent hydrogen or 3-12 membered heterocycloalkyl optionally substituted with one or two instances of R₂;
wherein said heterocycloalkyl comprises one or two oxygen atoms, one or two nitrogen atoms, one or two sulfur atoms, or any combination of two atoms selected from the group consisting of oxygen, nitrogen, and sulfur atoms; and each instance of R₂ independently represents —F, alkyl, haloalkyl, carbonyl, or alkoxy.

34. The method of claim 33, wherein said compound is selected from the group consisting of:

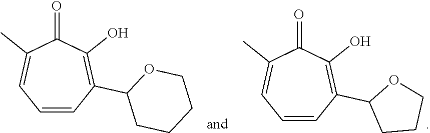 [SMILES: Cc1cccc(C2CCCCO2)c(O)c1=O ; Cc1cccc(C2CCCO2)c(O)c1=O]

35. The method of claim 33, wherein:
said heterocycloalkyl comprises one or two nitrogen atoms; and at least one of said nitrogen atoms is represented by N(R_{2b});
R_{2b} independently represents hydrogen, —C(O)R₅, or —C(O)OR₅; and
R₅ independently represents hydrogen, alkyl, or substituted alkyl.

36. The method of claim 30, wherein:
R_{b}, R_{c}, and R_{d} independently represent hydrogen or —OR₃; and
R₃ independently represents (C1-C15)alkyl, 3-12 membered cycloalkyl or bicycloalkyl, 3-12 membered heterocycloalkyl or heterobicylcloalkyl comprising one or two oxygen atoms, one or two nitrogen atoms, one or two sulfur atoms, or any combination of two atoms selected from the group consisting of oxygen, nitrogen, and sulfur atoms.

37. The method of claim 36, wherein each instance of R³ is optionally substituted with R₄; and R₄ independently represents alkyl, halogen-substituted alkyl, alkoxy, or hydroxy.

38. The method of any of claims 2-10, wherein Rₐ represents hydrogen.

39. The method of claim 38, wherein R_{b}, R_{c}, and R_{d} independently represent hydrogen, (C1-C15) alkyl, —F or —CF₃.

40. The compound of claim 38 or 39, wherein said compound is selected from the group consisting of:

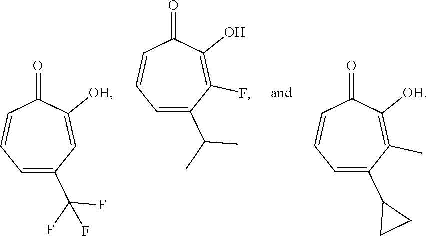

41. The method of claim 39, wherein said compound is selected from the group consisting of:

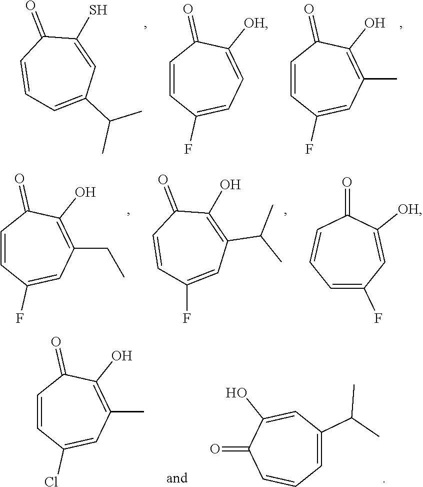

42. The method of claim 38, wherein R_{b}, R_{c}, and R_{d} independently represent hydrogen, (C1-C15)alkyl, —Cl, —F or —CF₃.

43. The method of claim 42, wherein said compound is

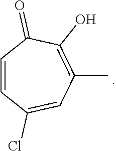

44. The method of claim 38, wherein:
R_{b}, R_{c}, and R_{d} independently represent hydrogen, 3-12 membered cycloalkyl or or 3-12 membered heterocycloalkyl each optionally substituted with one or two instances of R₂;
wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

45. The method of claim 44, wherein said compound is selected from the group consisting of:

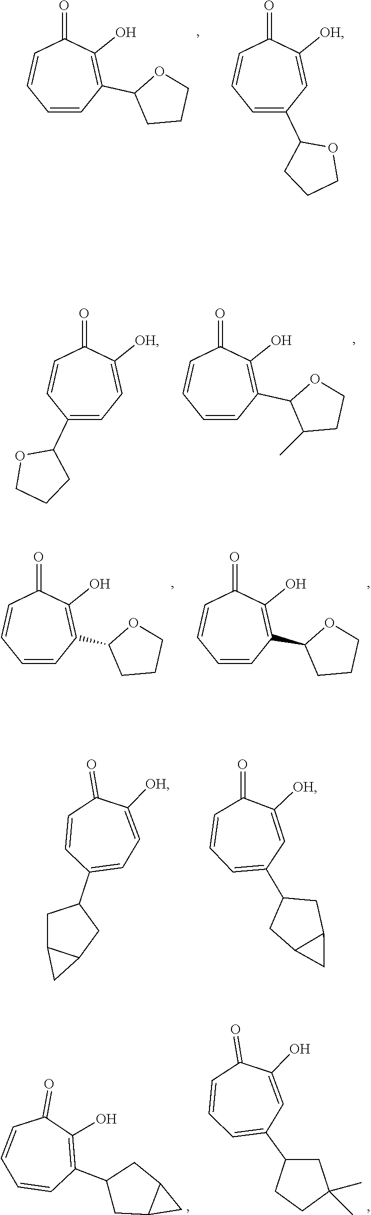

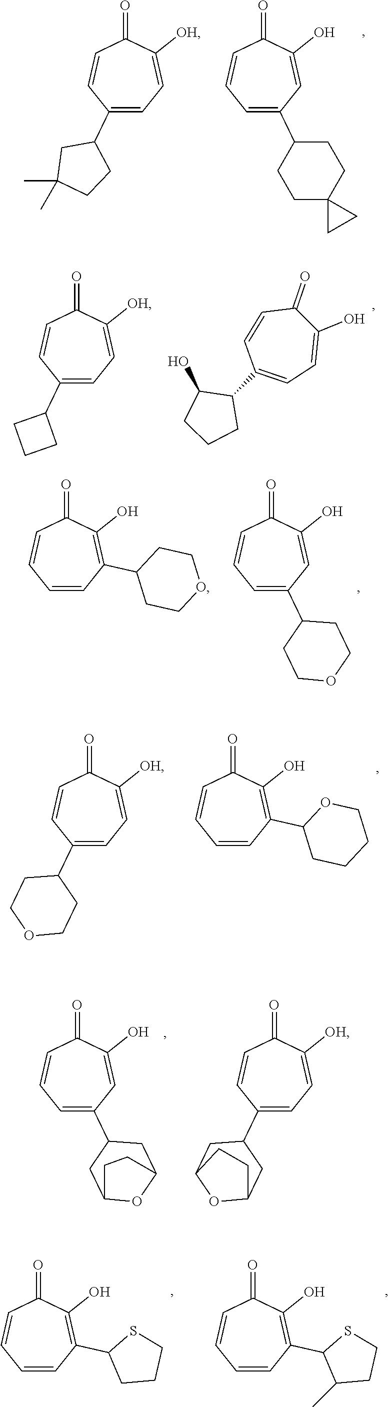

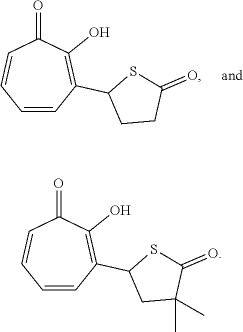 [SMILES: O=C1CCC(c2ccccc(=O)c2O)S1 ; CC1(C)CC(c2ccccc(=O)c2O)SC1=O]

46. The method of claim 45, wherein the compound is:

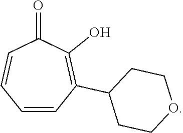 [SMILES: O=c1ccccc(C2CCOCC2)c1O]

47. The method of claim 45, wherein the compound is selected from:

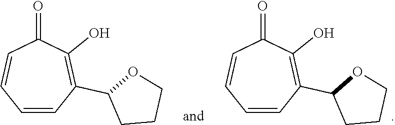

48. The method of claim 44, wherein said compound is selected from the group consisting of:

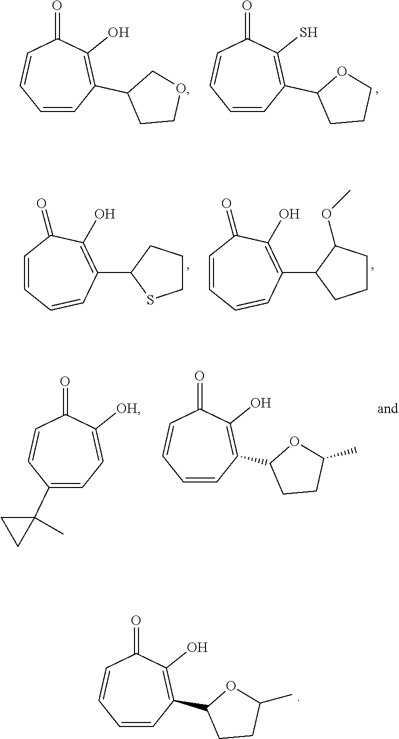

49. The method of claim 38, wherein:
R_{b} is halo;
R_{c} and R_{d} independently represent hydrogen, 3-12 membered cycloalkyl, 3-12 membered heterocycloalkyl, cycloalkyloxy, or heterocycloalkyloxy,
each cycloalkyl heterocycloalkyl, cycloalkyloxy, or heterocycloalkyloxy is optionally substituted with one or two instances of R₂;
wherein said heterocycloalkyl comprises one or two oxygen atoms, one or two nitrogen atoms, one or two sulfur atoms, or any combination of two atoms selected from the group consisting of oxygen, nitrogen, and sulfur atoms; and
each instance of R₂ independently represents —F, alkyl, haloalkyl, carbonyl, or alkoxy.

50. The method of claim 49, wherein said compound is selected from the group consisting of:

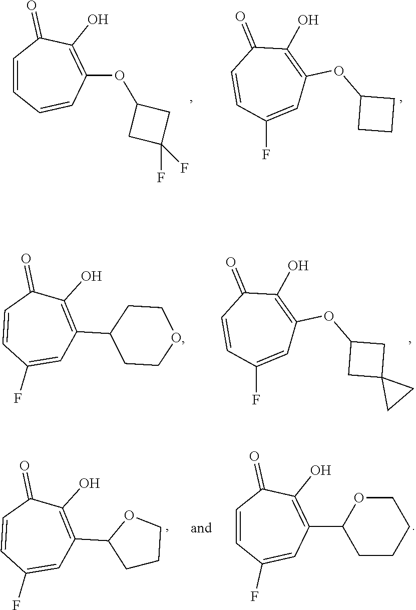 [SMILES: O=c1ccccc(OC2CC(F)(F)C2)c1O ; O=c1ccc(F)cc(OC2CCC2)c1O ; O=c1ccc(F)cc(C2CCOCC2)c1O ; O=c1ccc(F)cc(OC2CC3(CC3)C2)c1O ; O=c1ccc(F)cc(C2CCCO2)c1O ; O=c1ccc(F)cc(C2CCCCO2)c1O]

51. The method of claim 44, wherein:
said heterocycloalkyl comprises one or two nitrogen atoms; and at least one of said nitrogen atoms is represented by N(R_{2b});
R_{2b} independently represents hydrogen, —C(O)R₅, or —C(O)OR₅; and
R₅ independently represents hydrogen, alkyl, or substituted alkyl.

52. The method of claim 51, wherein said compound is selected from the group consisting of:

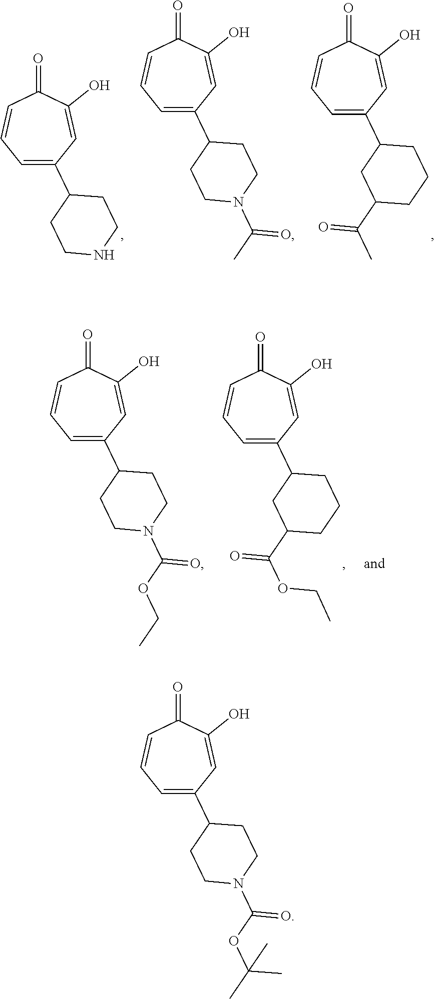

53. The method of claim 38, wherein:
R_{b}, R_{c}, and R_{d} independently represent hydrogen or —OR₃; and
R₃ independently represents (C1-C15) alkyl, 3-12 membered cycloalkyl or bicycloalkyl, or 3-12 membered heterocycloalkyl or heterobicylcloalkyl comprising one or two oxygen atoms, one or two nitrogen atoms, one or two sulfur atoms, or any combination of two atoms selected from the group consisting of oxygen, nitrogen, and sulfur atoms.

54. The method of claim 53, wherein said compound is selected from the group consisting of:

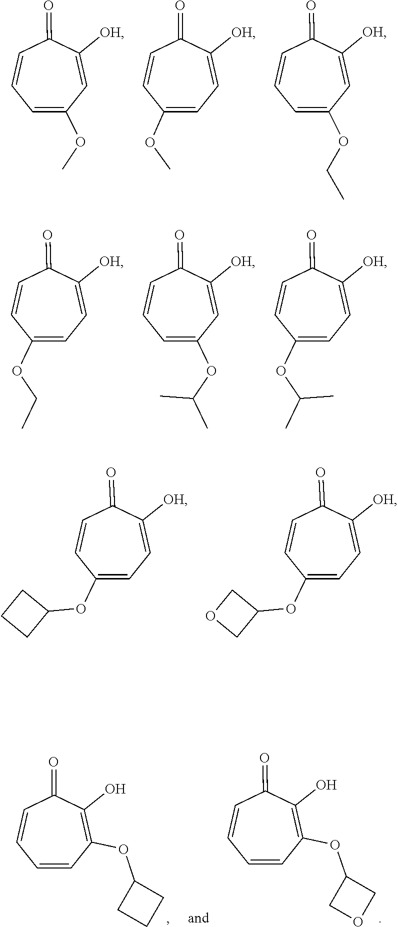

55. The method of claim 50, wherein the compound is:

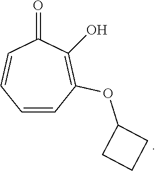 [SMILES: O=c1ccccc(OC2CCC2)c1O]

56. The method of claim 53, wherein each instance of R₃ is optionally substituted with R₄; and R₄ independently represents alkyl, halogen-substituted alkyl, alkoxy, or hydroxy.

57. The method of claim 53, wherein said compound is selected from the group consisting of:

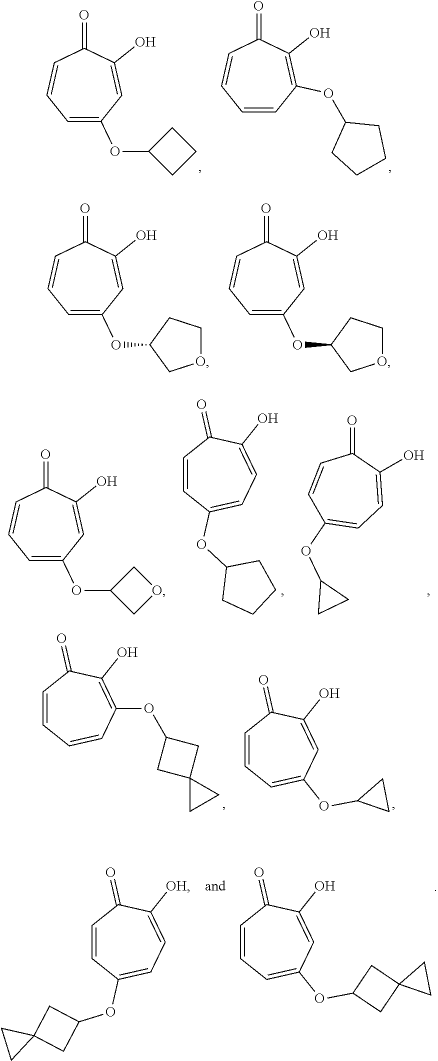

58. The method of claim 38, wherein:
R_{b}, R_{c}, and R_{d} independently represent hydrogen or -alkyl-R₃ or —O-alkyl-R₃; and
R₃ independently represents aryl, substituted aryl, 3-12 membered cycloalkyl or bicycloalkyl, or a 3-12 membered heterocycloalkyl or heterobicylcloalkyl comprising one or two oxygen atoms, one or two nitrogen atoms, one or two sulfur atoms, or any combination of two atoms selected from the group consisting of oxygen, nitrogen, and sulfur atoms.

59. The method of claim 58, wherein said compound is selected from the group consisting of:

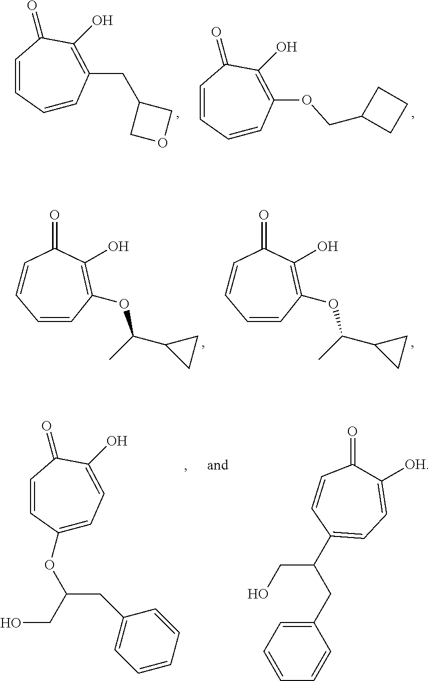

60. The method of claim 38 or 39, wherein Rₐ′ is halo or alkyl.

61. The method of claim 58, wherein said compound is

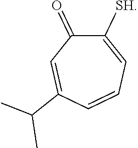

62. A method of treating a disease or condition selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA, comprising administering to a subject in need thereof a therapeutically effective amount of a compound, or a pharmaceutically acceptable salt thereof, having a structure represented by Formula IIa, Formula IIb, or Formula IIc: 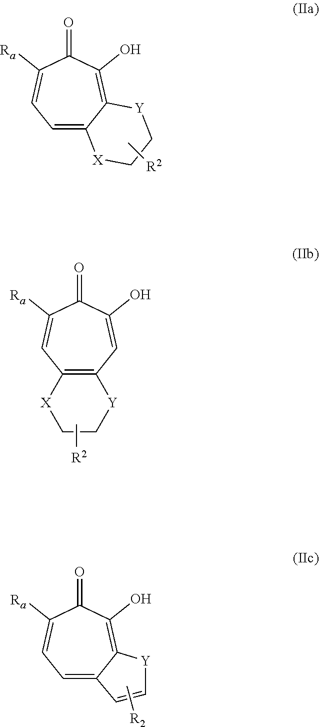
wherein:
Rₐ represents hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl;
X and Y independently represent O, S, NH, or CR₅R₆;
R₂ represents —F, alkyl, haloalkyl, or alkoxy; and
R₅ and R₆ represent independently for each occurrence H, (C1-C15) alkyl, or substituted (C1-C15)alkyl;
provided the compound is not

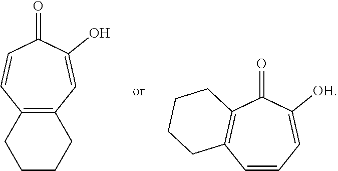

63. The method of claim 61, wherein the compound is represented by Formula Ila or Formula IIb.

64. The method of claim 63, wherein Rₐ represents hydrogen.

65. The method of claim 64, wherein said compound is selected from the group consisting of:

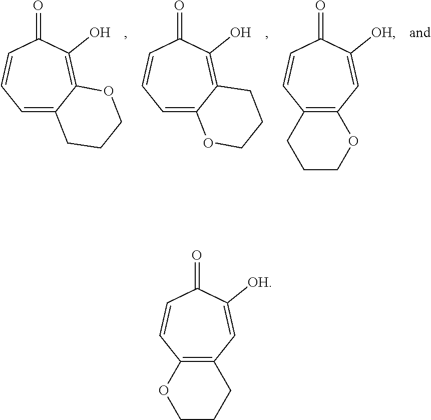

66. The method of claim 64, wherein said compound is

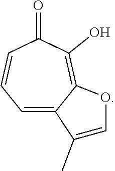

67. A method of treating a disease or condition selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA, comprising administering to a subject in need thereof a therapeutically effective amount of a compound, or a pharmaceutically acceptable salt thereof, having a structure according to Formula (Ic): 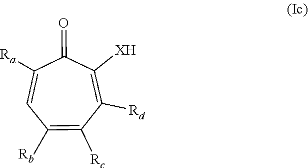
wherein:
X represents sulfur or oxygen;
Rₐ, R_{b}, R_{c}, and R_{d} independently represent hydrogen, halo, alkyl, substituted alkyl, heteroalkyl, alkoxy, substituted alkoxy, alkoxyalkyl, substituted alkoxyalkyl, aryloxy, substituted aryloxy, heteroaryloxy, substituted heteroaryloxy, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, heteroalkynyl, aryl, substituted aryl, heteroaryl, or substituted heteroaryl;
at least one of Rₐ, R_{b}, R_{c}, and R_{d} is aryloxy, substituted aryloxy, heteroaryloxy, substituted heteroaryloxy, aryl, substituted aryl, heteroaryl, substituted heteroaryl; and
provided that Rₐ, R_{b}, R_{c}, and R_{d} are not all hydrogen.

68. The method of claim 67, wherein at least one of Rₐ, R_{b}, R_{c}, and R_{d} is aryl, substituted aryl, heteroaryl, substituted heteroaryl.

69. The method of claim 67, wherein at least one of R_{b}, R_{c}, and R_{d} is aryl, substituted aryl, heteroaryl, substituted heteroaryl.

70. The compound of claim 67, represented by Formula (Id): 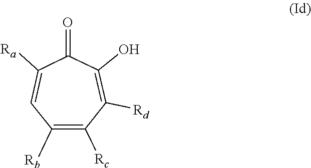
wherein:
Rₐ, R_{b}, R_{c}, and R_{d} independently represent hydrogen, halo, alkyl, substituted alkyl, heteroalkyl, alkoxy, substituted alkoxy, alkoxyalkyl, substituted alkoxyalkyl, cycloalkyl, substituted cycloalkyl, heterocycloalkyl, substituted heterocycloalkyl, alkenyl, substituted alkenyl, heteroalkenyl, cycloalkenyl, substituted cycloalkenyl, heterocycloalkenyl, substituted heterocycloalkenyl, alkynyl, substituted alkynyl, heteroalkynyl, aryl, substituted aryl, heteroaryl, substituted heteroaryl;
at least one of R_{b}, R_{c}, and R_{d} is aryl, substituted aryl, heteroaryl, or substituted heteroaryl; and
provided that Rₐ, R_{b}, R_{c}, and R_{d} are not all hydrogen.

71. The method of any one of claims 67-70, wherein:
each of R_{b}, R_{c}, and R_{d} that is aryl, substituted aryl, heteroaryl or substituted heteroaryl is represented by Formula II: 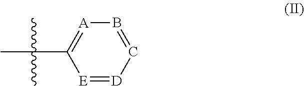
each of A, B, C, D, and E independently represents CH, N, or CR;
for each instance of Formula II the total number of nitrogen atoms among A, B, C, D, and E is 0, 1, or 2; and
each instance of R independently represents halo, alkyl, substituted alkyl, heteroalkyl, substituted heteroalkyl, cycloalkyl, substituted cycloalkyl, heterocycloalkyl, substituted heterocycloalkyl, hydroxy, alkoxy, substituted alkoxy, alkoxyalkyl, substituted alkoxyalkyl, cycloalkoxy, substituted cycloalkoxy, cyano, amino, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl.

72. The method of claim 71, wherein each instance of R independently represents chloro, fluoro, bromo, iodo, cyano, trifluoromethyl, amino, hydroxy, (C1-C12)alkyl, (C3-C12)cycloalkyl, (C1-C12)alkoxy, (C3-12)cycloalkoxy, or (C3-C12)heterocycloalkyl; and
heterocycloalkyl comprises one or two oxygen atoms, one or two nitrogen atoms, one or two sulfur atoms, or any combination of two atoms selected from the group consisting of oxygen, nitrogen, and sulfur atoms.

73. The method of claim 71 or 72, wherein:
Rₐ, R_{b}, and R_{d} represent hydrogen; and
R_{c} represents an aryl, substituted aryl, heteroaryl, or substituted heteroaryl according to Formula II.

74. The compound of claim 73, wherein said compound is selected from the group consisting of:

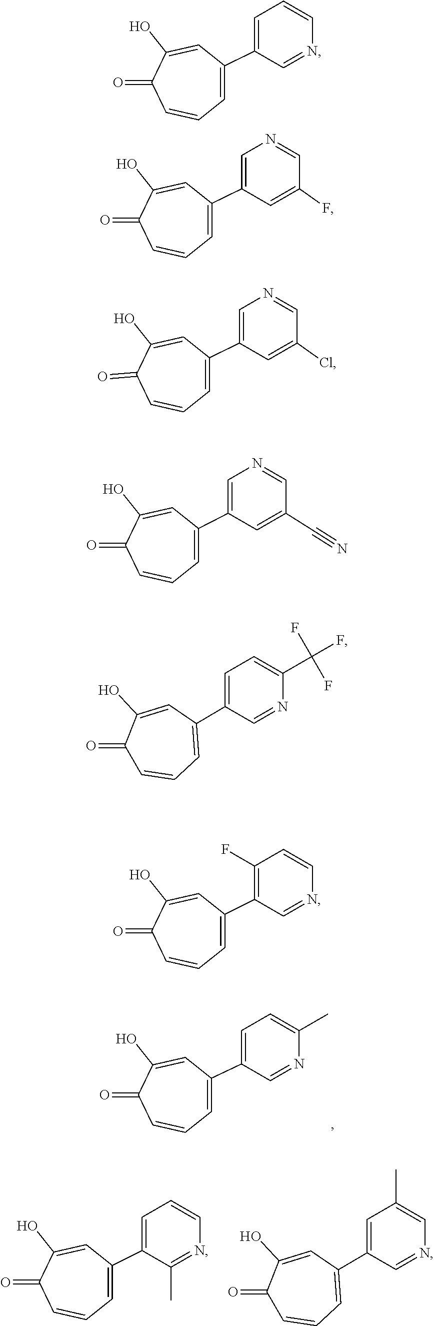

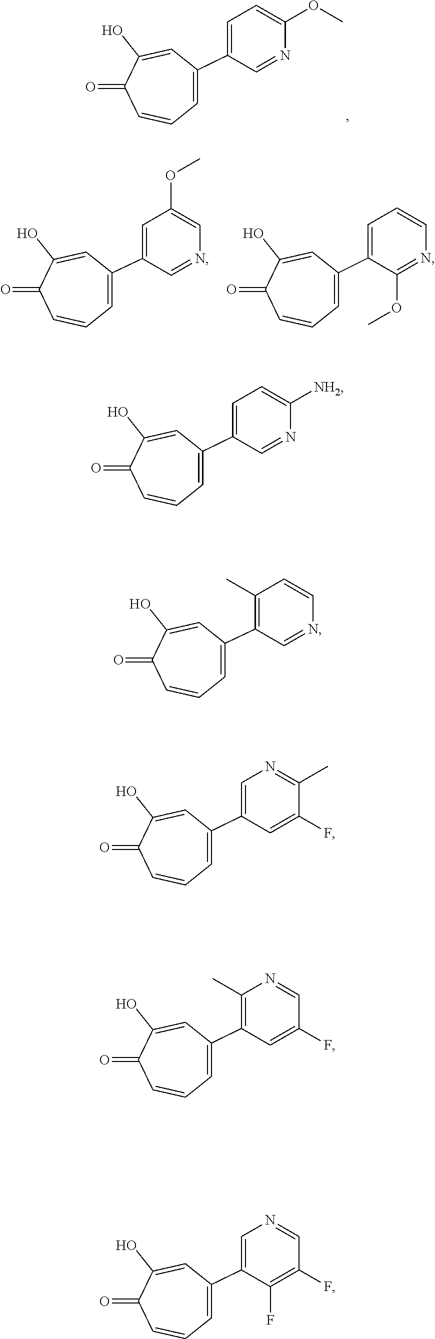

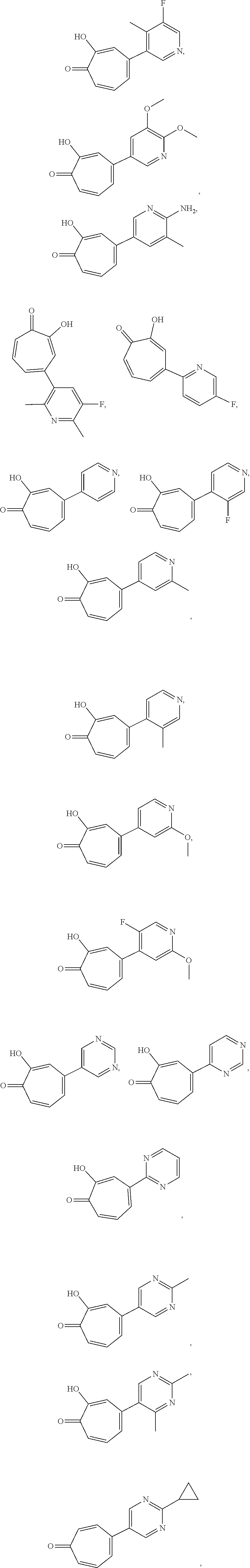

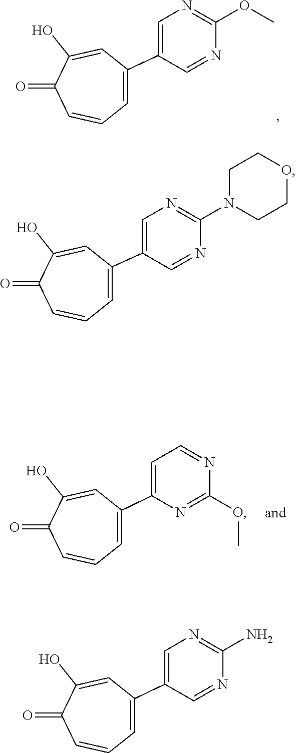 [SMILES: COc1ncc(-c2cccc(=O)c(O)c2)cn1 ; O=c1cccc(-c2cnc(N3CCOCC3)nc2)cc1O ; COc1nccc(-c2cccc(=O)c(O)c2)n1 ; "and" ; Nc1ncc(-c2cccc(=O)c(O)c2)cn1]

75. The method of claim 71 or 72, wherein:
Rₐ, R_{c}, and R_{d} represent hydrogen; and
R_{b} represents an aryl, substituted aryl, heteroaryl, or substituted heteroaryl according to Formula II.

76. The method of claim 75, wherein said compound is selected from the group consisting of:

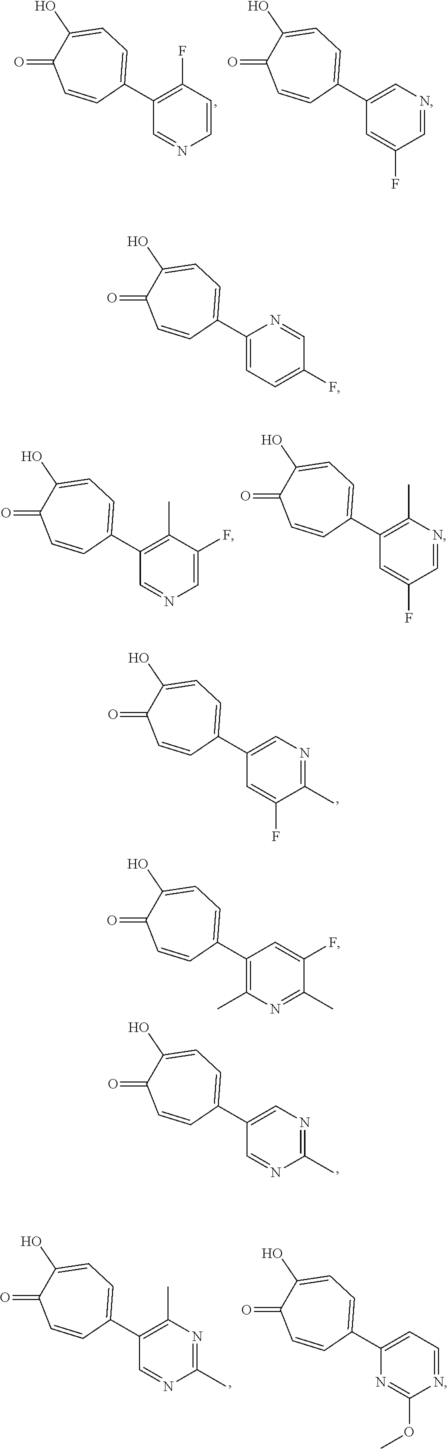

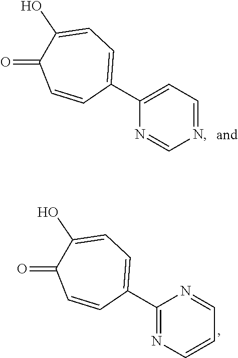 [SMILES: O=c1ccc(-c2ccncn2)ccc1O ; O=c1ccc(-c2ncccn2)ccc1O]

77. The method of claim 71 or 72, wherein:
Rₐ, R_{b}, and R_{c} represent hydrogen; and
R_{d} represents an aryl, substituted aryl, heteroaryl, or substituted heteroaryl according to Formula II.

78. The method of claim 77, wherein said compound is selected from the group consisting of:

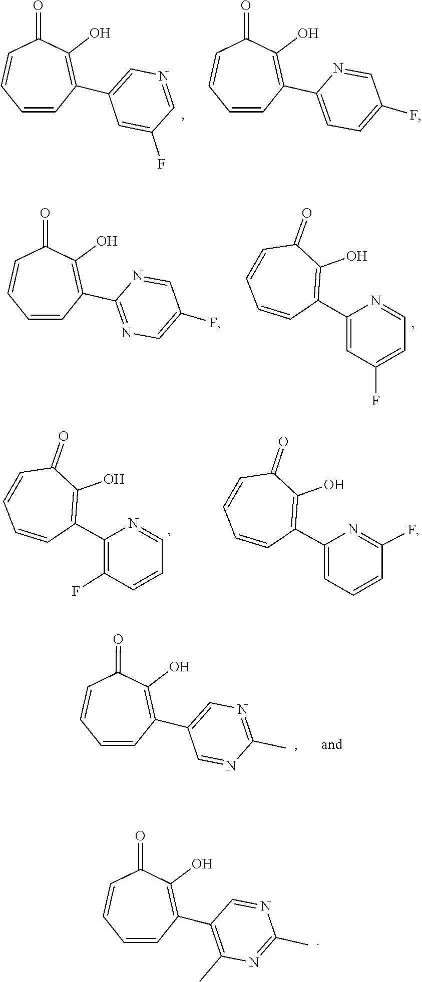

79. The method of claim 78, wherein said compound is selected from the group consisting of:

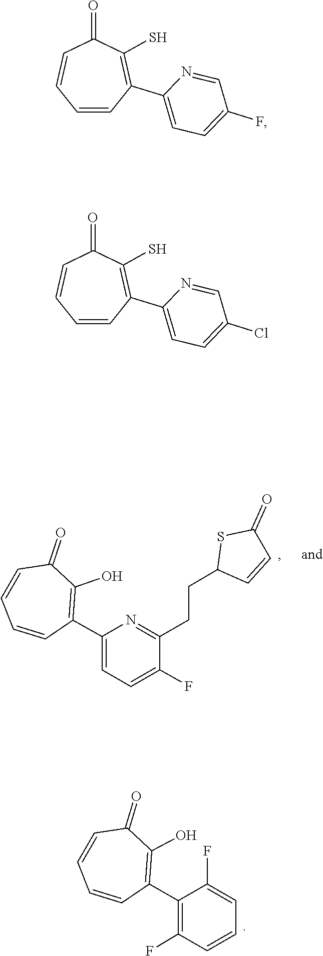 [SMILES: O=c1ccccc(-c2ccc(F)cn2)c1S ; O=c1ccccc(-c2ccc(Cl)cn2)c1S ; O=C1C=CC(CCc2nc(-c3ccccc(=O)c3O)ccc2F)S1 ; O=c1ccccc(-c2c(F)cccc2F)c1O]

80. The method of claim 71 or 72, wherein:
Rₐ represents alkyl;
one and only one of R_{b}, R_{c}, and R_{d} represents aryl, substituted aryl, heteroaryl, or substituted heteroaryl according to Formula II; and
two of R_{b}, R_{c}, and R_{d} represent hydrogen.

81. The method of claim 80, wherein said compound is selected from the group consisting of:

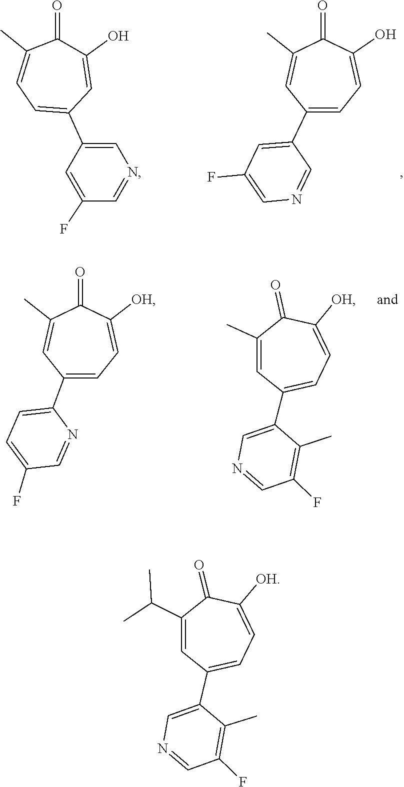

82. The method of claim 80 or 81, wherein:
Rₐ represents an aryl, substituted aryl, heteroaryl, or substituted heteroaryl according to Formula II;
one and only one of R_{b}, R_{c}, and R_{d} represents alkyl; and
two of R_{b}, R_{c}, and R_{d} represent hydrogen.

83. The method of claim 82, wherein said compound is selected from the group consisting of:

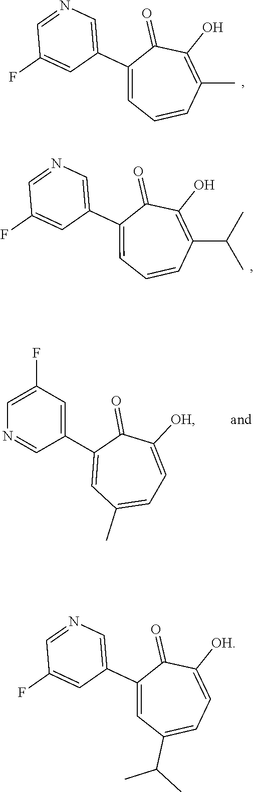 [SMILES: Cc1cccc(-c2cncc(F)c2)c(=O)c1O ; CC(C)c1cccc(-c2cncc(F)c2)c(=O)c1O ; Cc1ccc(O)c(=O)c(-c2cncc(F)c2)c1 ; "and" ; CC(C)c1ccc(O)c(=O)c(-c2cncc(F)c2)c1]

84. The method of claim 71 or 72, wherein:
Rₐ represents an aryl, substituted aryl, heteroaryl, or substituted heteroaryl according to Formula II; and
each of R_{b}, R_{c}, and R_{d} represent hydrogen.

85. The method of claim 84, wherein said compound is selected from the group consisting of:

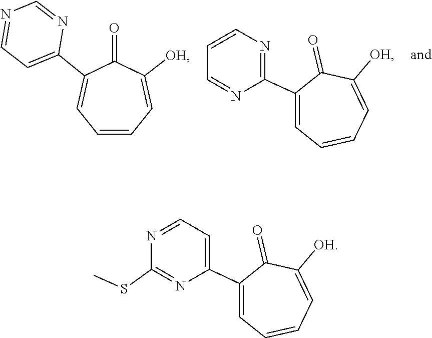

86. The method of claim 67, wherein at least one of Rₐ, R_{b}, R_{c}, and R_{d} is aryloxy, substituted aryloxy, heteroaryloxy, or substituted heteroaryloxy.

87. The method of claim 67, wherein at least one of R_{b}, R_{c}, and R_{d} is aryloxy, substituted aryloxy, heteroaryloxy, or substituted heteroaryloxy.

88. The method of claim 86 or 87, wherein:
each of R_{b}, R_{c}, and R_{d} that is aryloxy, substituted aryloxy, heteroaryloxy, or substituted heteroaryloxy is represented by Formula IId 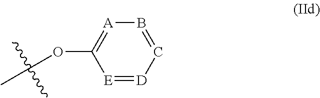
each of A, B, C, D, and E independently represents CH, N, or CR;
for each instance of Formula IId the total number of nitrogen atoms among A, B, C, D, and E is 0, 1, or 2; and
each instance of R independently represents halo, alkyl, substituted alkyl, heteroalkyl, substituted heteroalkyl, cycloalkyl, substituted cycloalkyl, heterocycloalkyl, substituted heterocycloalkyl, hydroxy, alkoxy, substituted alkoxy, alkoxyalkyl, substituted alkoxyalkyl, cycloalkoxy, substituted cycloalkoxy, cyano, amino, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl.

89. The method of claim 88, wherein each instance of R independently represents chloro, fluoro, bromo, iodo, cyano, trifluoromethyl, amino, hydroxy, (C1-C12)alkyl, (C3-C12)cycloalkyl, (C1-C12)alkoxy, (C3-12)cycloalkoxy, or (C3-C12)heterocycloalkyl; and
heterocycloalkyl comprises one or two oxygen atoms, one or two nitrogen atoms, one or two sulfur atoms, or any combination of two atoms selected from the group consisting of oxygen, nitrogen, and sulfur atoms.

90. The method of claim 88 or 89, wherein:
Rₐ, R_{b}, and Rₐ represent hydrogen; and
R_{c} represents an aryloxy, substituted aryloxy, heteroaryloxy, or substituted heteroaryloxy according to Formula IId.

91. The method of claim 88 or 89, wherein:
Rₐ, R_{c}, and R_{d} represent hydrogen; and
R_{b} represents an aryloxy, substituted aryloxy, heteroaryloxy, or substituted heteroaryloxy according to Formula IId.

92. The method of claim 88 or 89, wherein:
Rₐ, R_{b}, and R_{c} represent hydrogen; and
R_{d} represents an aryloxy, substituted aryloxy, heteroaryloxy, or substituted heteroaryloxy according to Formula IId.

93. The method of claim 88 or 89, wherein:
Rₐ represents alkyl;
one and only one of R_{b}, R_{c}, and R_{d} represents an aryloxy, substituted aryloxy, heteroaryloxy, or substituted heteroaryloxy according to Formula IId; and
two of R_{b}, R_{c}, and R_{d} represent hydrogen.

94. The method of claim 88 or 89, wherein:
Rₐ represents an aryl, substituted aryl, heteroaryl, or substituted heteroaryl according to Formula IId; and
each of R_{b}, R_{c}, and R_{d} represent hydrogen.

95. The method of claim 91 or 92, wherein said compound is selected from the group consisting of:

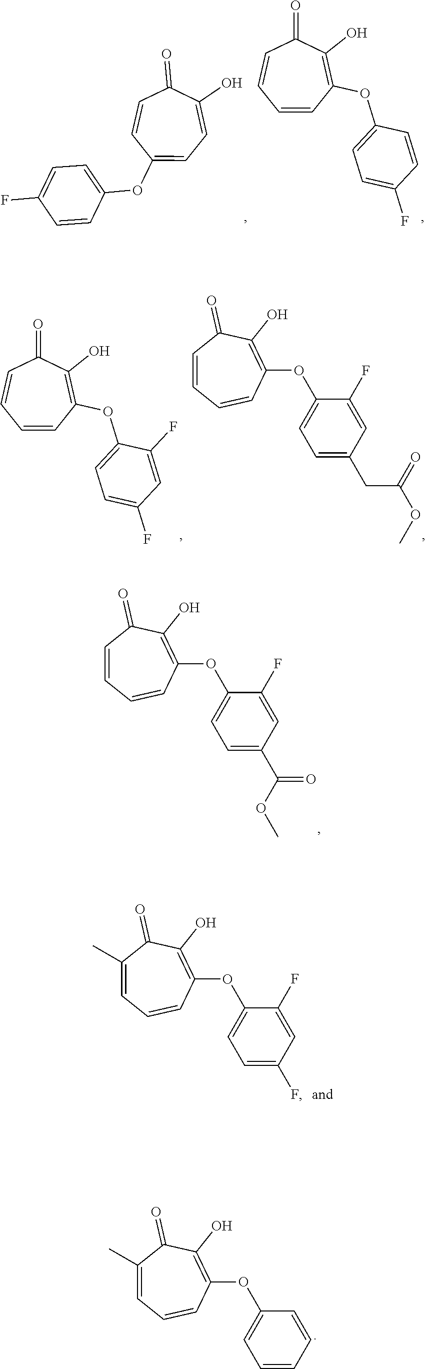

96. The method of any one of claims 67-70, wherein:
each of R_{b}, R_{c}, and R_{d} that is heteroaryl or substituted heteroaryl is represented by Formula IIe: 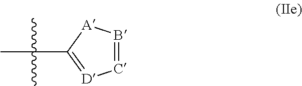
A′ represents O or S;
each of B′, C′, and D′ independently represents CH, N, or CR; and
each instance of R independently represents halo, alkyl, substituted alkyl, heteroalkyl, substituted heteroalkyl, cycloalkyl, substituted cycloalkyl, heterocycloalkyl, substituted heterocycloalkyl, hydroxy, alkoxy, substituted alkoxy, alkoxyalkyl, substituted alkoxyalkyl, cycloalkoxy, substituted cycloalkoxy, cyano, amino, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl.

97. The method of any one of claims 67-70, wherein:
each of R_{b}, R_{c}, and R_{d} that is heteroaryl or substituted heteroaryl is represented by by Formula IIf: 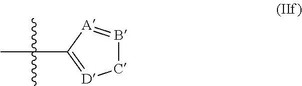
C′ represents O or S;
each of A′, B′, and D′ independently represents CH, N, or CR; and
each instance of R independently represents halo, alkyl, substituted alkyl, heteroalkyl, substituted heteroalkyl, cycloalkyl, substituted cycloalkyl, heterocycloalkyl, substituted heterocycloalkyl, hydroxy, alkoxy, substituted alkoxy, alkoxyalkyl, substituted alkoxyalkyl, cycloalkoxy, substituted cycloalkoxy, cyano, amino, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl.

98. The method of any one of claims 67-70, wherein:
each of R_{b}, R_{c}, and R_{d} that is heteroaryl or substituted heteroaryl is represented by by Formula IIg: 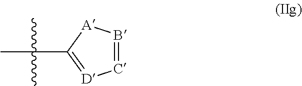
D′ represents O or S;
each of A′, B′, and C′ independently represents CH, N, or CR; and
each instance of R independently represents halo, alkyl, substituted alkyl, heteroalkyl, substituted heteroalkyl, cycloalkyl, substituted cycloalkyl, heterocycloalkyl, substituted heterocycloalkyl, hydroxy, alkoxy, substituted alkoxy, alkoxyalkyl, substituted alkoxyalkyl, cycloalkoxy, substituted cycloalkoxy, cyano, amino, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl.

99. The method of any one of claims 57-60, wherein:
each of R_{b}, R_{c}, and R_{d} that is heteroaryl or substituted heteroaryl is represented by by Formula IIh: 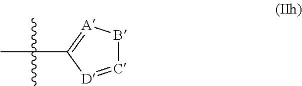
B′ represents O or S;
each of A′, C′, and D′ independently represents CH, N, or CR; and
each instance of R independently represents halo, alkyl, substituted alkyl, heteroalkyl, substituted heteroalkyl, cycloalkyl, substituted cycloalkyl, heterocycloalkyl, substituted heterocycloalkyl, hydroxy, alkoxy, substituted alkoxy, alkoxyalkyl, substituted alkoxyalkyl, cycloalkoxy, substituted cycloalkoxy, cyano, amino, alkenyl, substituted alkenyl, heteroalkenyl, cycloalkenyl, substituted cycloalkenyl, heterocycloalkenyl, substituted heterocycloalkenyl, alkynyl, substituted alkynyl, or heteroalkynyl.

100. The method of any one of claims 97-99, wherein each instance of R independently represents chloro, fluoro, bromo, iodo, cyano, trifluoromethyl, amino, hydroxy, (C1-C12)alkyl, (C3-C12)cycloalkyl, (C1-C12)alkoxy, (C3-12)cycloalkoxy, or (C3-C12)heterocycloalkyl; and
heterocycloalkyl comprises one or two oxygen atoms, one or two nitrogen atoms, one or two sulfur atoms, or any combination of two atoms selected from the group consisting of oxygen, nitrogen, and sulfur atoms.

101. The method of any one of claims 97-100, wherein:
Rₐ, R_{b}, and R_{d} represent hydrogen; and
R_{c} represents a heteroaryl or substituted heteroaryl according to any one of Formulas IIe-IIh.

102. The method of any one of claims 97-100, wherein:
Rₐ, R_{c}, and R_{d} represent hydrogen; and
R_{b} represents a heteroaryl or substituted heteroaryl according to any one of Formulas IIe-IIh.

103. The method of any one of claims 97-100, wherein:
Rₐ, R_{b}, and R_{c} represent hydrogen; and
Rₐ represents a heteroaryl or substituted heteroaryl according to any one of Formulas IIe-IIh.

104. The method of any one of claims 97-100, wherein:
Rₐ and R_{c} represent hydrogen;
R_{b} represents halo, alkyl or substituted alkyl;
R_{d} represents a heteroaryl or substituted heteroaryl according to any one of Formulas IIe-IIh.

105. The method of any one of claims 97-100, wherein:
Rₐ represents halo or alkyl;
one and only one of R_{b}, R_{c}, and R_{d} represents a heteroaryl or substituted heteroaryl according to any one of Formulas IIe-IIh; and
two of R_{b}, R_{c}, and R_{d} represent hydrogen.

106. The method of any one of claims 97-100, wherein:
Rₐ represents a heteroaryl or substituted heteroaryl according to any one of Formulas IIe-IIh;
one and only one of R_{b}, R_{c}, and R_{d} represents alkyl; and
two of R_{b}, R_{c}, and R_{d} represent hydrogen.

107. The method of any one of claims 97-100, wherein:
Rₐ represents a heteroaryl or substituted heteroaryl according to any one of Formulas IIe-IIh; and
each of R_{b}, R_{c}, and R_{d} represent hydrogen.

108. The method of any one of claims 101-107, wherein said compound or tautomer is selected from the group consisting of:

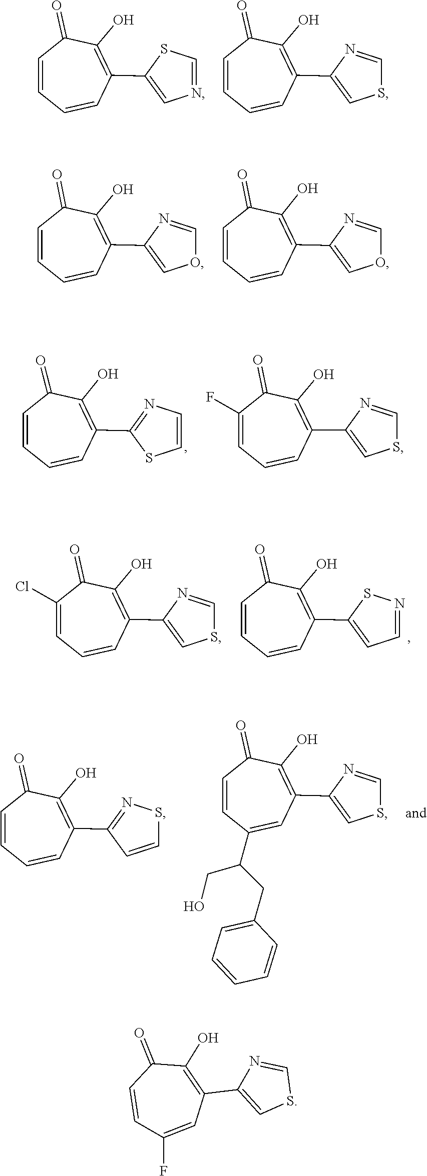

109. A method of treating a disease or condition selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA, comprising administering to a subject in need thereof a therapeutically effective amount of a compound, or a pharmaceutically acceptable salt thereof, having a structure of Formula (I): 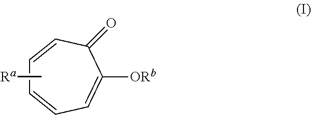
or a salt thereof; wherein R^{a} is C₁₋₂₀-alkyl, C₂₋₂₀-alkenyl, C₂₋₂₀-alkynyl, C₃₋₉-cycloalkyl, aryl, or heteroaryl, each of which is unsubstituted or substituted with a substituent selected from the group consisting of halo, NO₂, CN, C₁₋₆-alkyl, C₁₋₆-haloalkyl, and C₁₋₆-alkoxy; and
R^{b} is hydrogen or methyl; provided the compound is not hinokitiol.

110. The method of claim 109, selected from the group having a structure of: 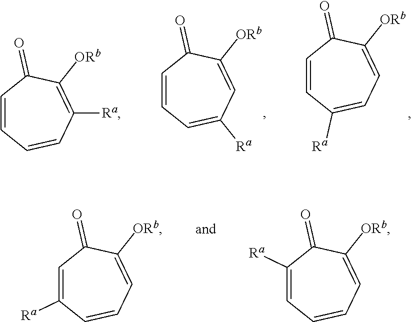
and pharmaceutically acceptable salts thereof.

111. The method of any one of claims 109-110, wherein R^{a} is C₁₋₄-alkyl, C₂₋₄-alkenyl, C₂₋₄-alkynyl, or C₃₋₄-cycloalkyl; provided the compound is not hinokitiol.

112. The method of any one of claims 109-110, wherein R^{a} is selected from the group consisting of:

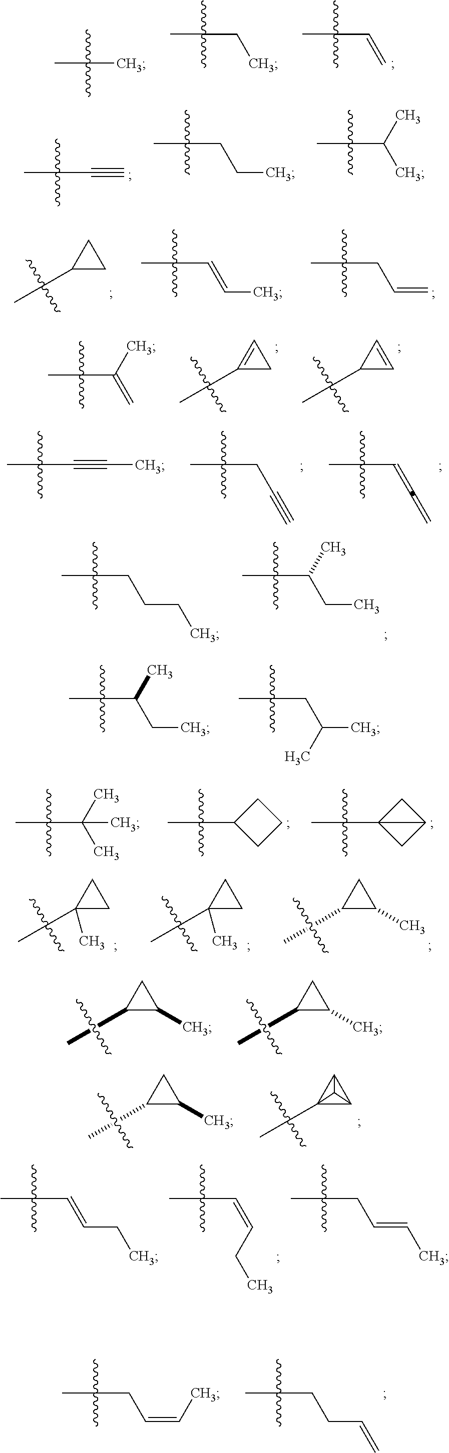

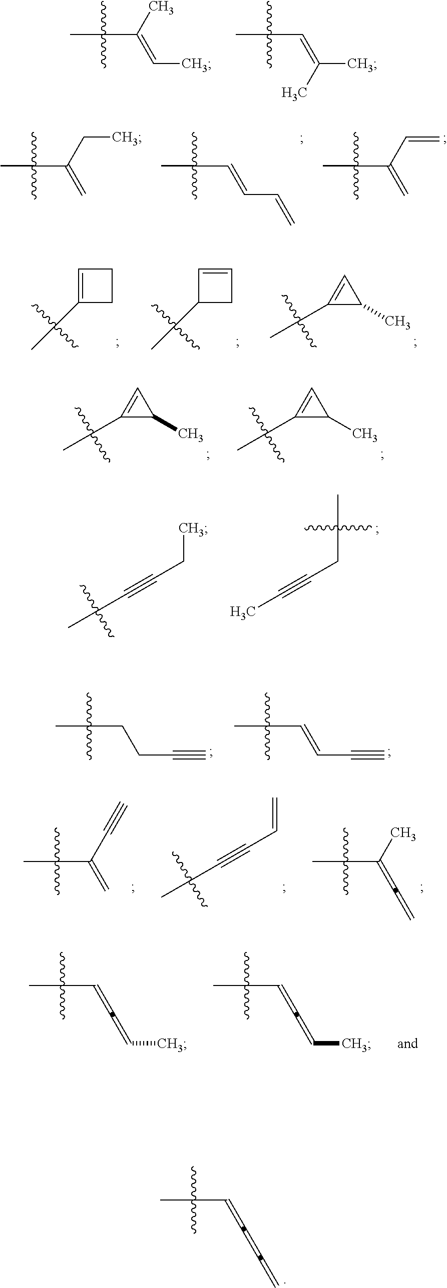

113. The method of any one of claims 109-110, wherein R^{a} is selected from the group consisting of:

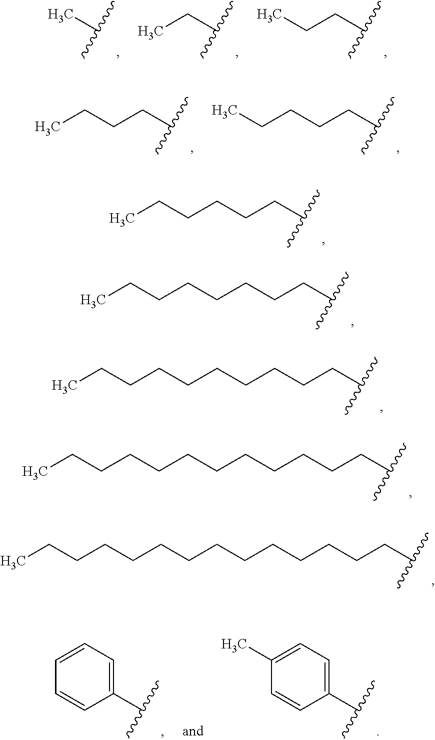

114. The method of any one of claims 109-110, wherein R^{a} is selected from the group consisting of:

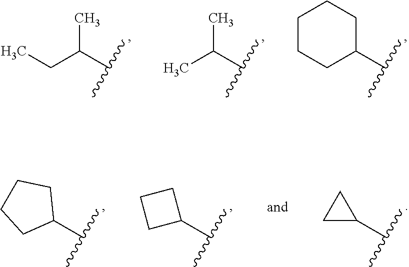

115. A method of treating a disease or condition selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA, comprising administering to a subject in need thereof a therapeutically effective amount of a compound, or a pharmaceutically acceptable salt thereof, having a structure selected from:

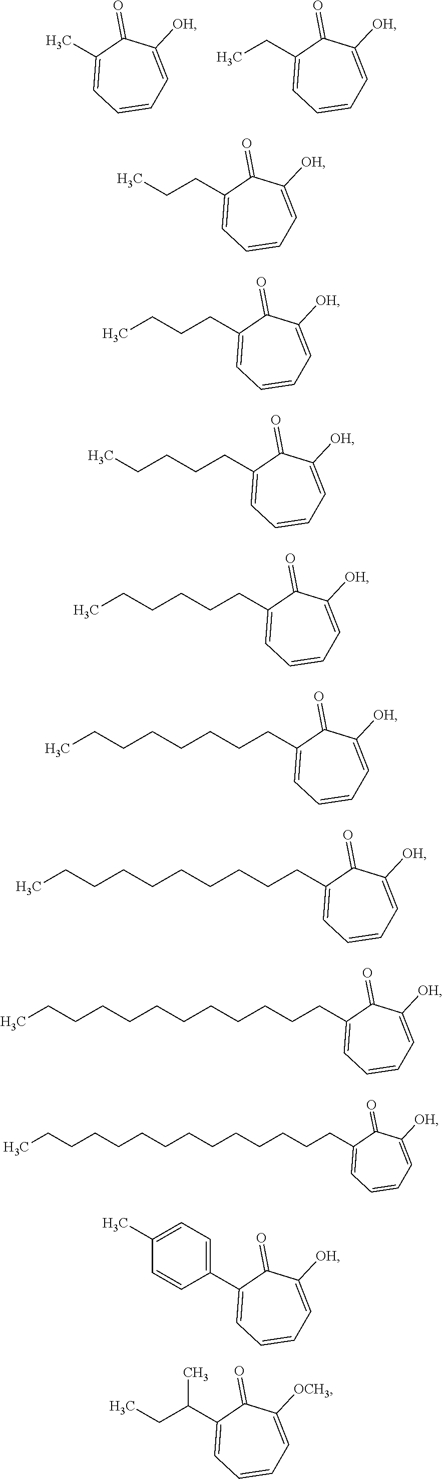

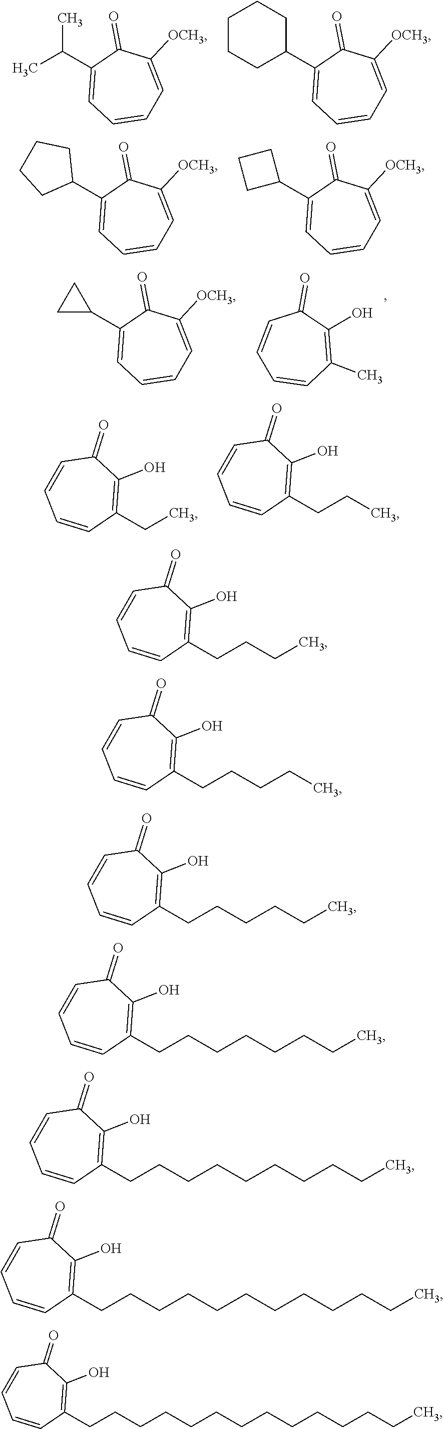

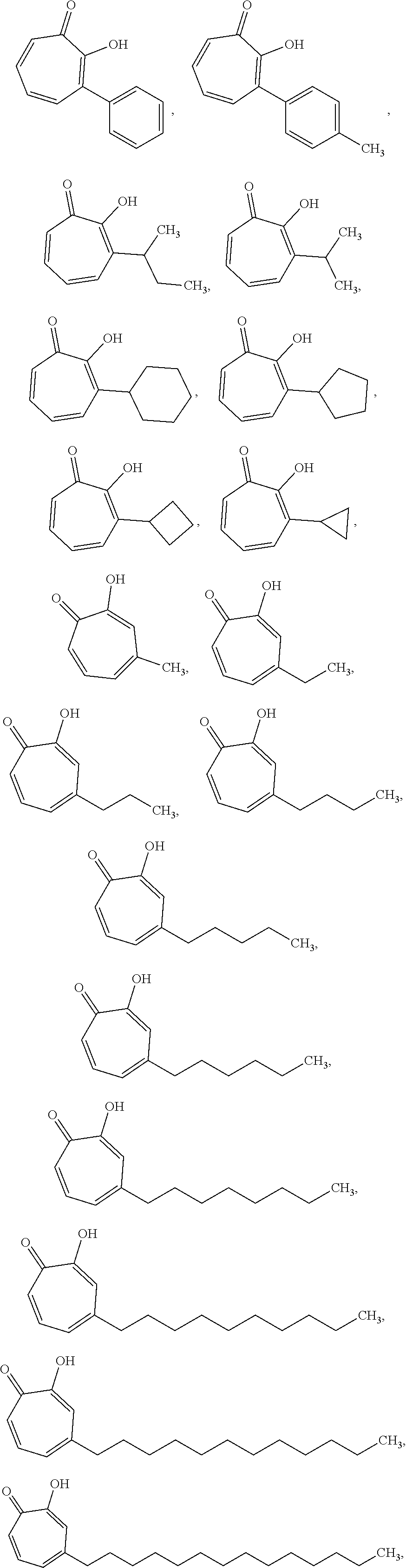

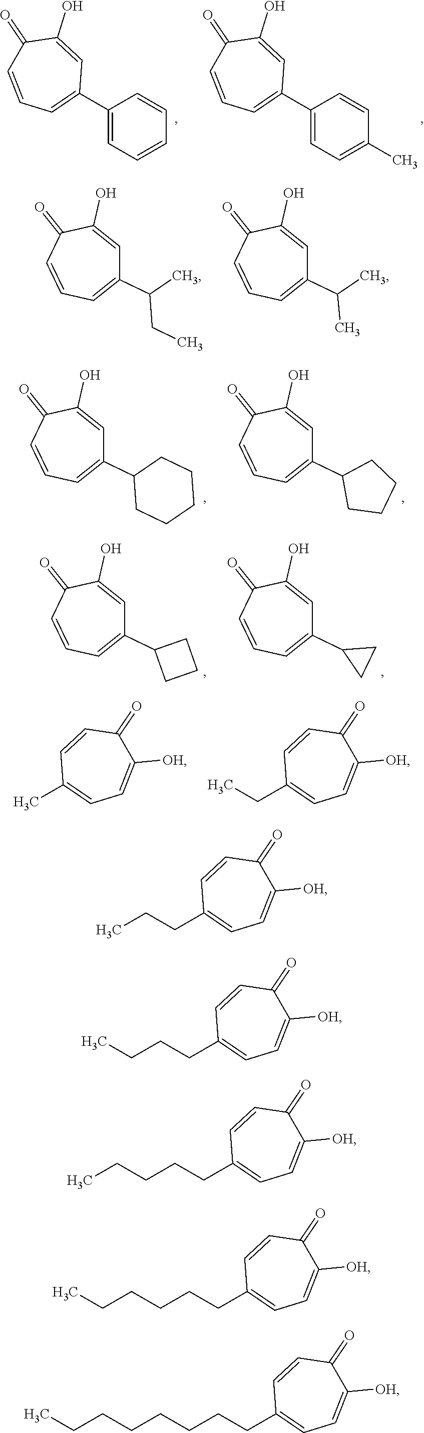

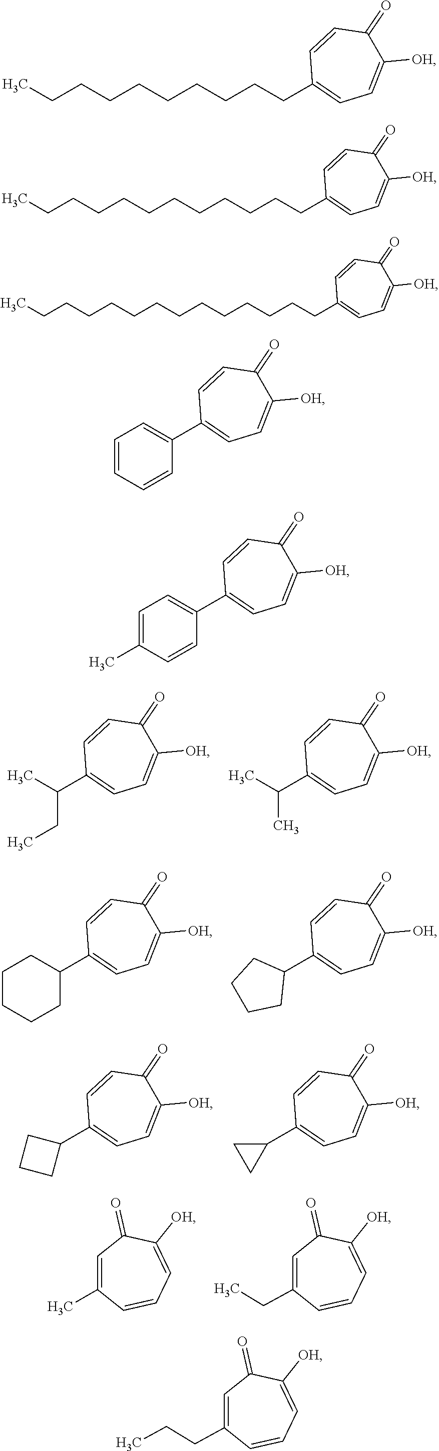

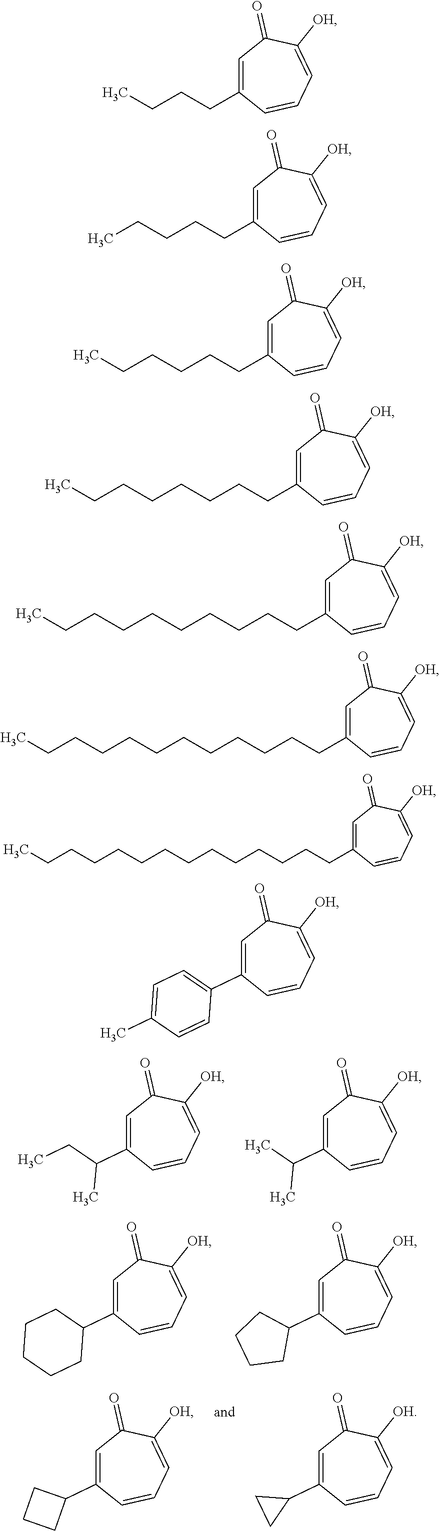

116. The method of claim 115, wherein the compound is selected from: 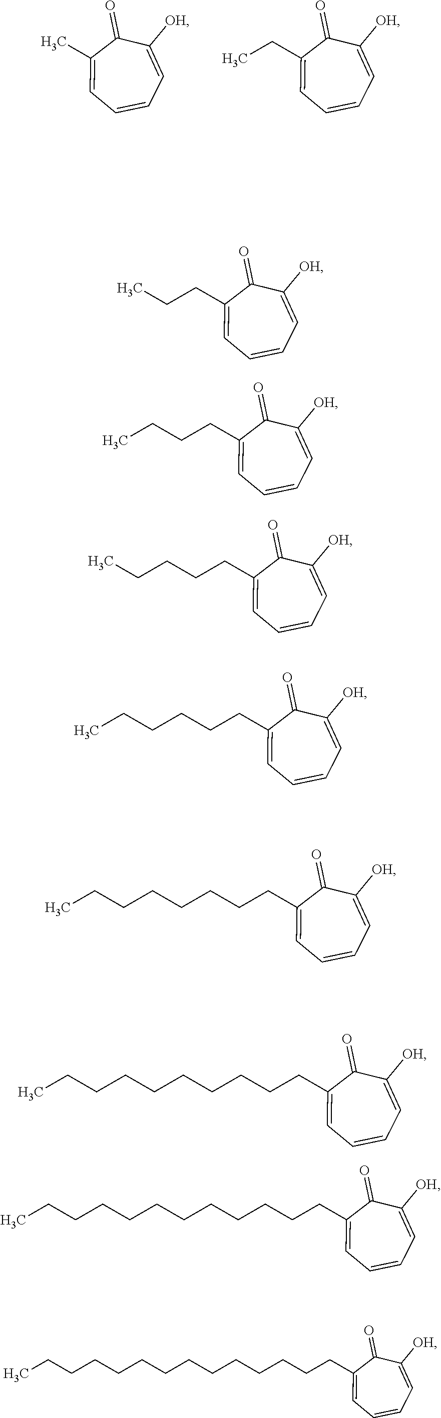 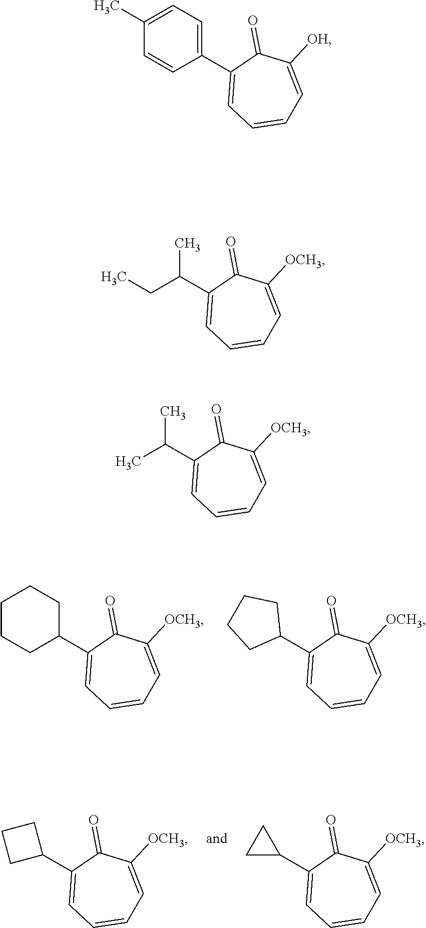
or a pharmaceutically acceptable salt thereof.

117. The method of claim 115, wherein the compound is selected from: 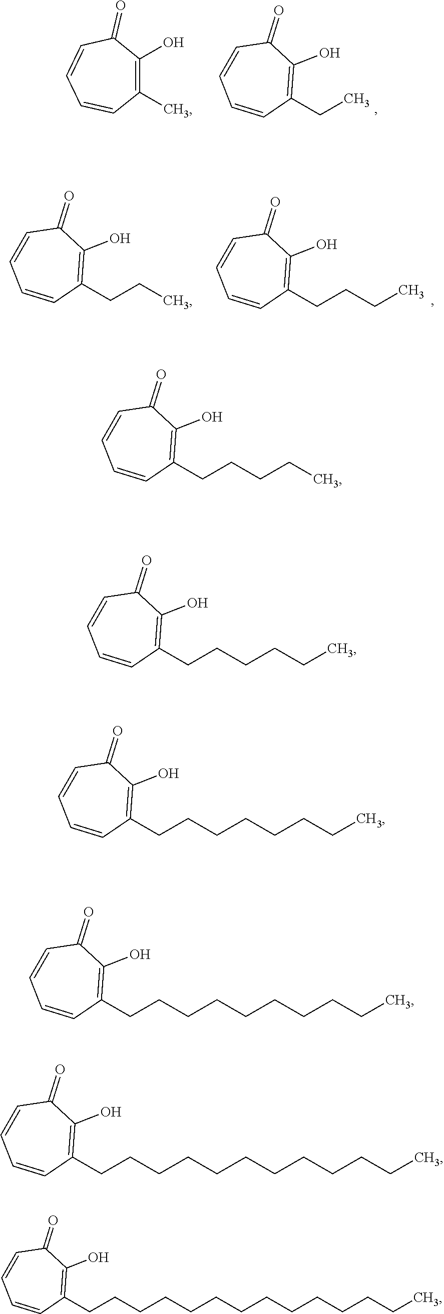 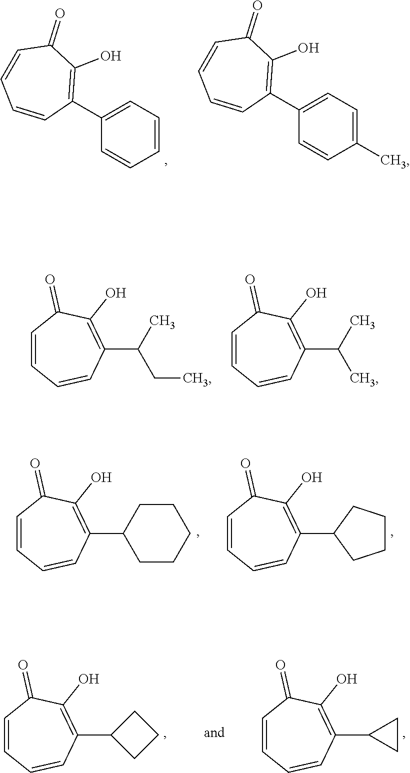
or a pharmaceutically acceptable salt thereof.

118. The method of claim 115, wherein the compound is selected from: 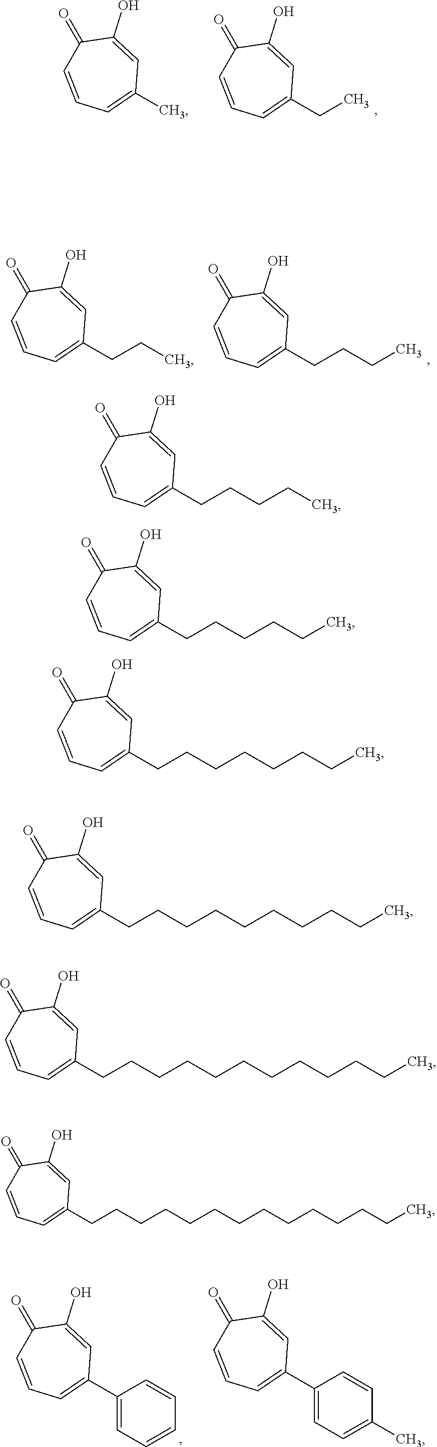 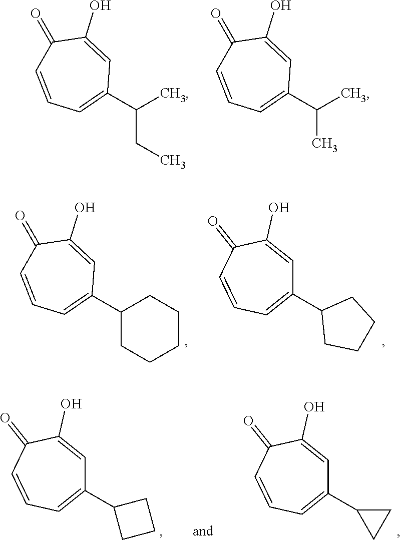
or a pharmaceutically acceptable salt thereof.

119. The method of claim 115, wherein the compound is selected from: 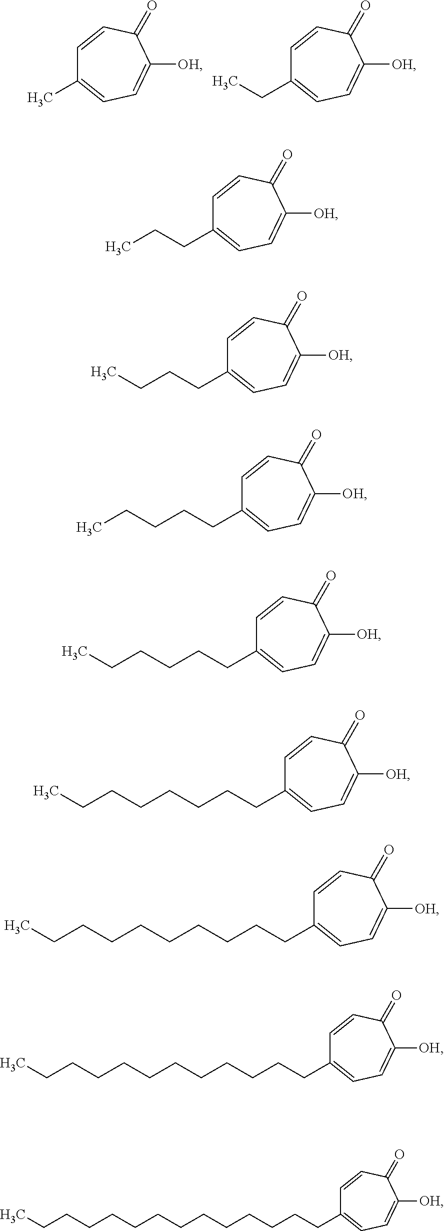 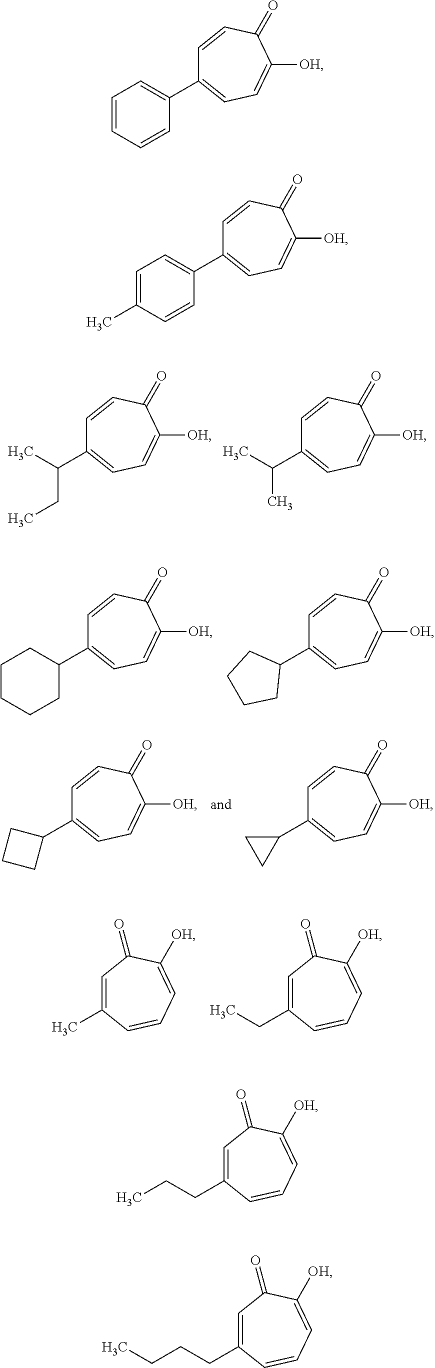 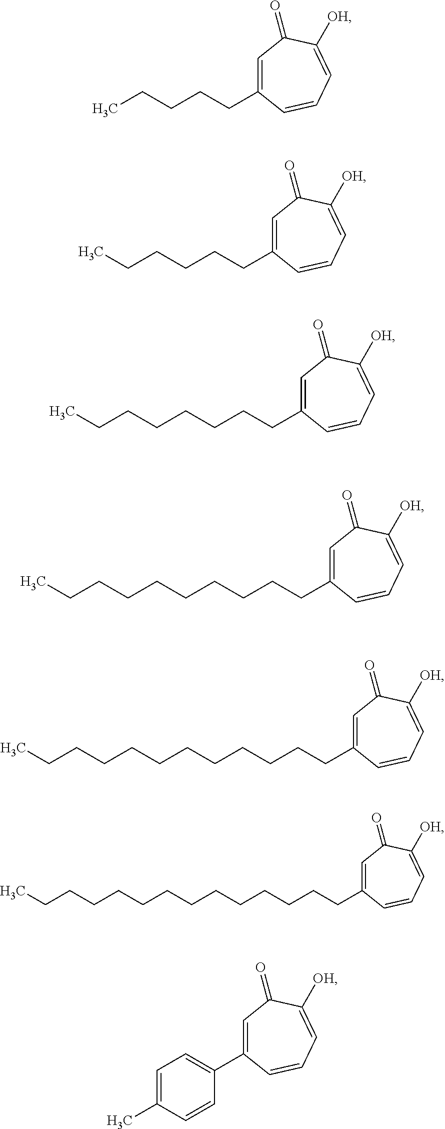 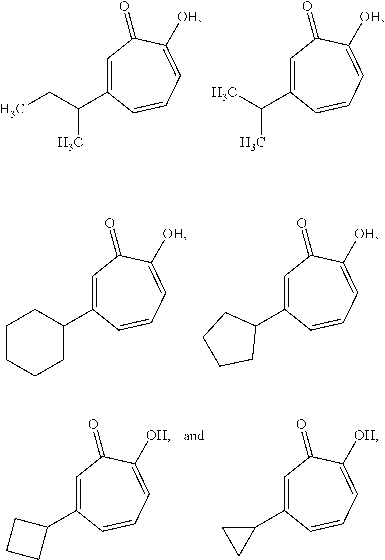
or a pharmaceutically acceptable salt thereof.

120. A method of treating a disease or condition selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA, comprising administering to a subject in need thereof a therapeutically effective amount of a compound, or a pharmaceutically acceptable salt thereof, having a structure selected from the group:

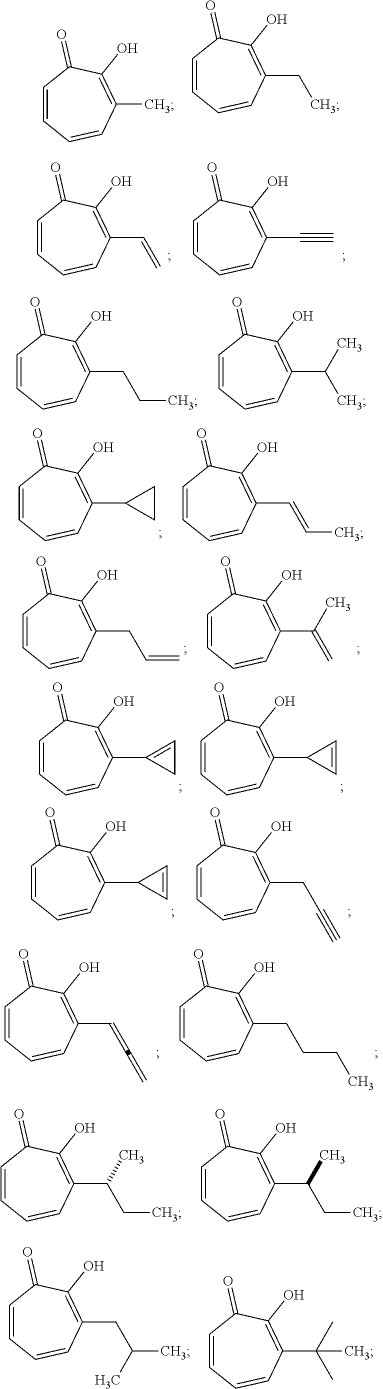

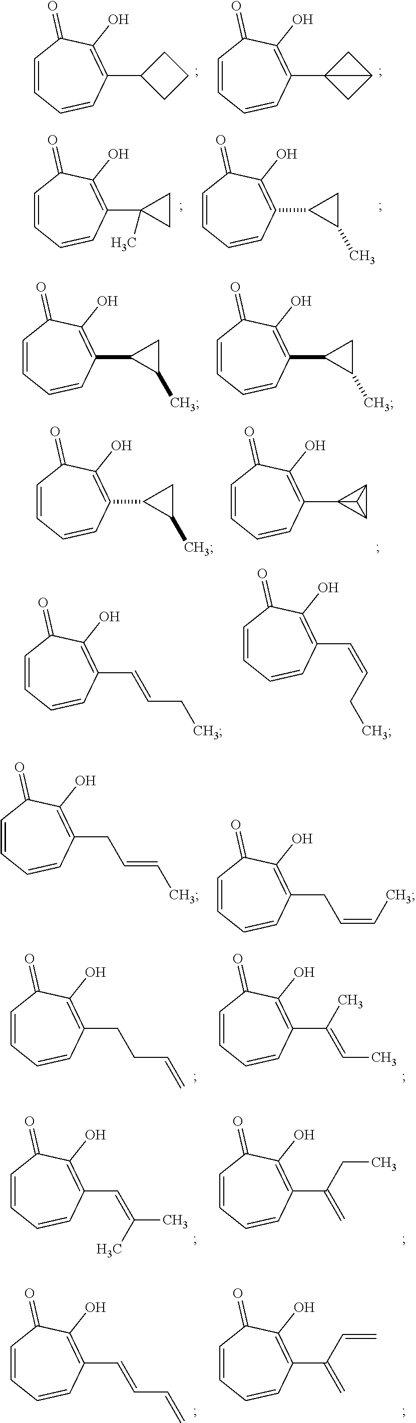

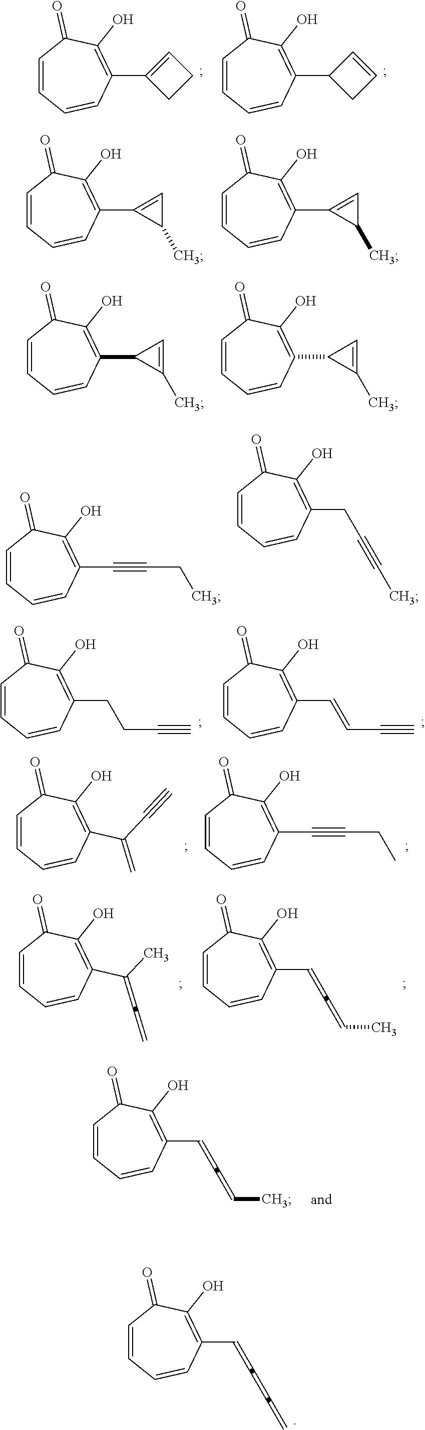

121. A method of treating a disease or condition selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA, comprising administering to a subject in need thereof a therapeutically effective amount of a compound, or a pharmaceutically acceptable salt thereof, having a structure selected from the group consisting of:

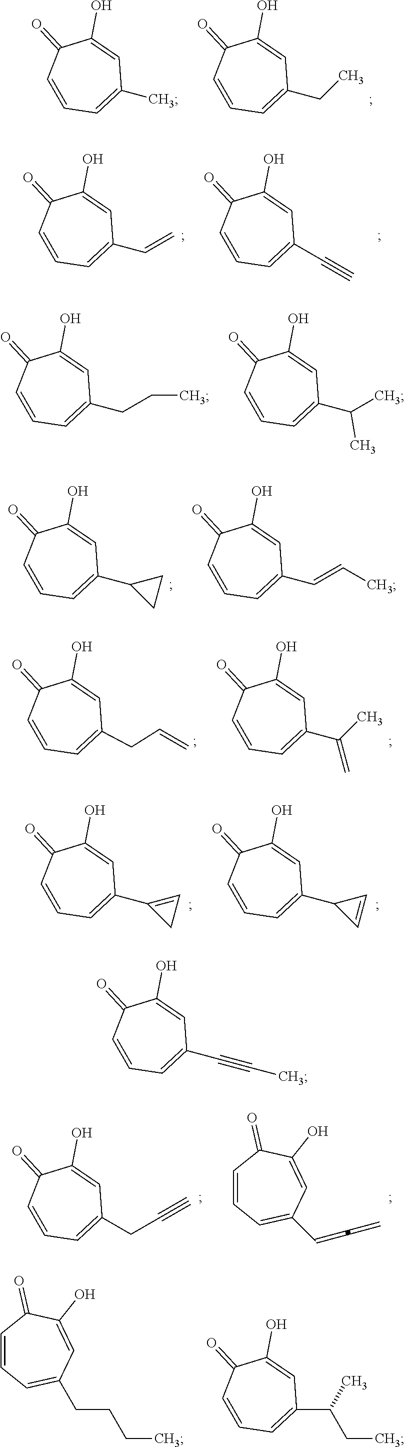

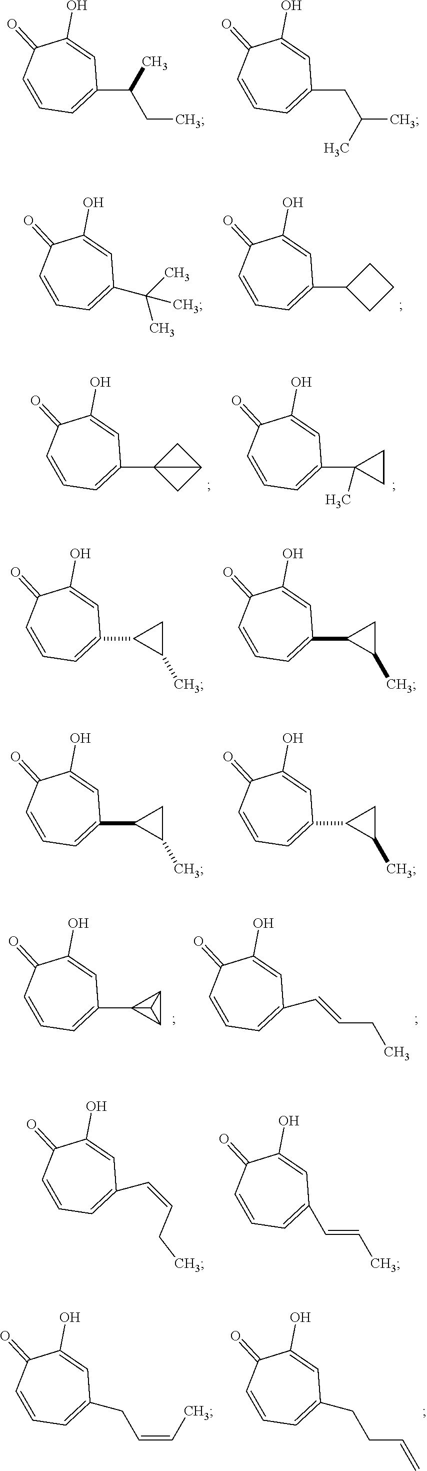

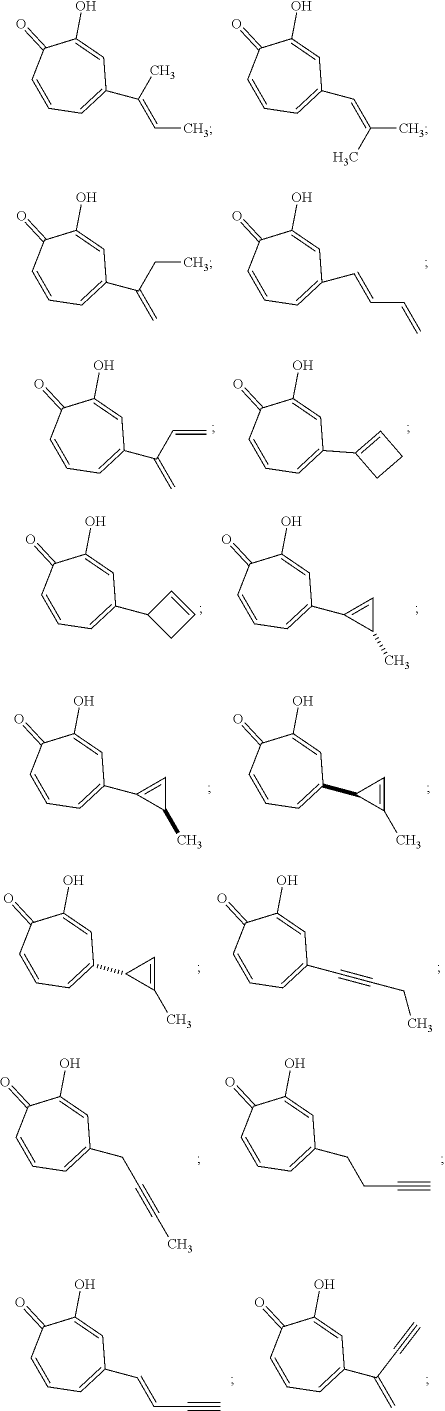

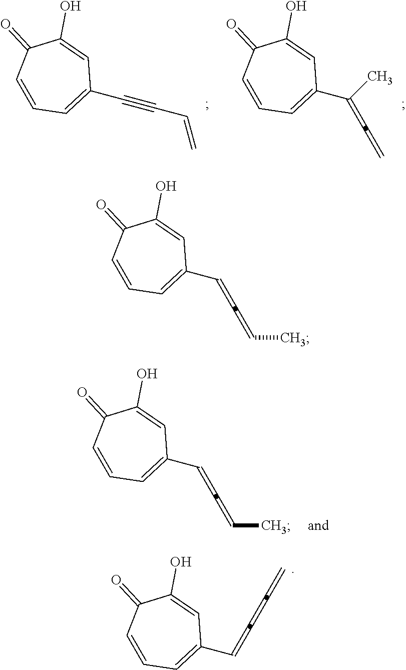

122. A method of treating a disease or condition selected from the group consisting of an inflammatory disorder leading to an abnormal suppression of ferroportin production, a neurodegeneration with brain iron accumulation (NBIA), a Beta-propeller Protein-associated Neurodegeneration (BPAN), a Pantothenate Kinase-associated Neurodegeneration (PKAN), a PLA2G6-associated Neurodegeneration (PLAN), a Mitochondrial-membrane Protein-associated Neurodegeneration (MPAN), a Fatty Acid Hydroxylase-Associated Neurodegeneration (FAHN), a COASY Protein-Associated Neurodegeneration (CoPAN), a Aceruloplasminemia, a Kufer-Rakeb Syndrome, a Parkinson's Disease 9 (PARK9), a Neuroferritinopathy, a Woodhouse-Sakati Syndrome, and an Idiopathic NBIA, comprising administering to a subject in need thereof a therapeutically effective amount of a compound, or a pharmaceutically acceptable salt thereof, selected from the group consisting of:

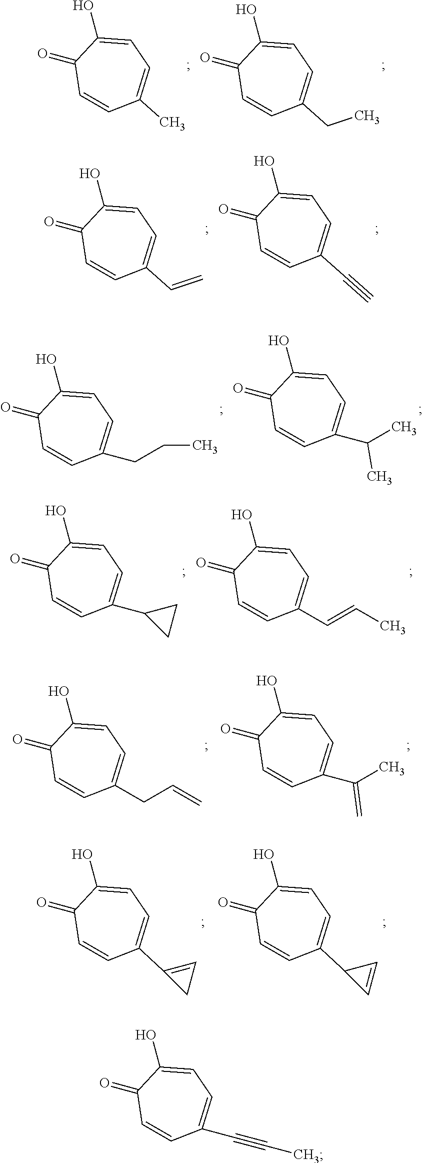

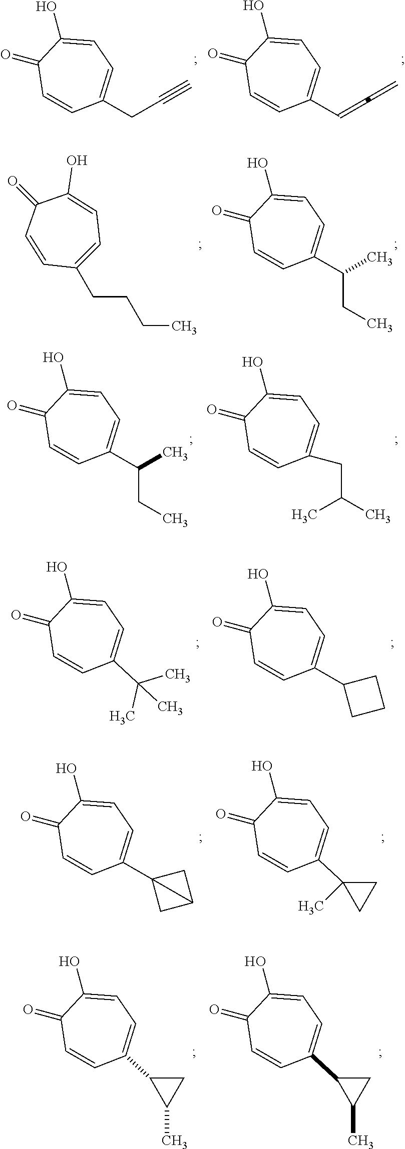

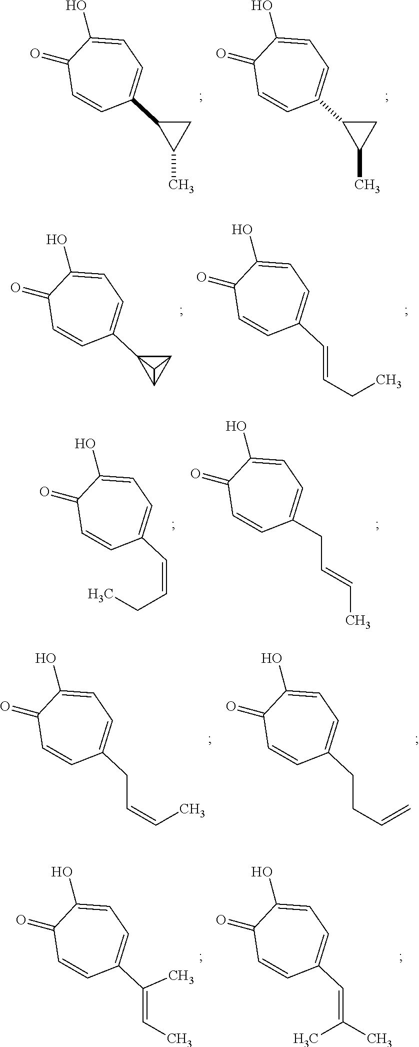

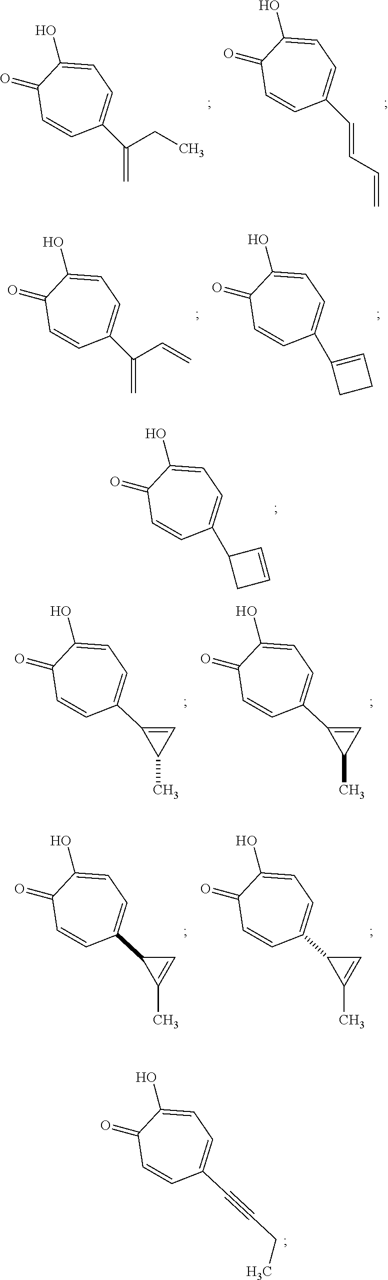

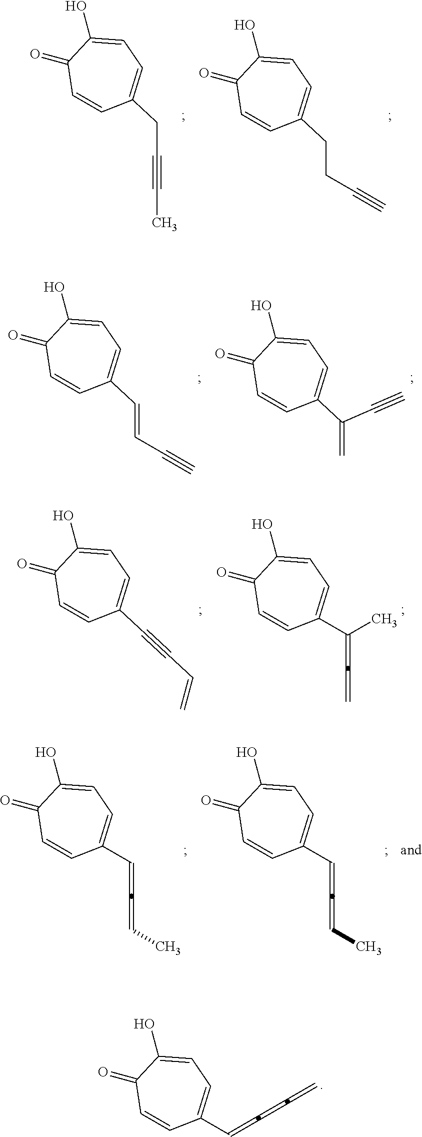

123. The method of any one of claims 1-122, further comprising administering to the subject a therapeutically effective amount of a chelator.

124. The method of claim 123, wherein the chelator is deferasirox or deferiprone or a combination of them.

125. The method of any one of claims 1-124, wherein the disease or condition is aceruloplasminemia.

126. The method of any one of claims 1-125, wherein the disease or condition is selected from the group consisting of Huntington's disease, amyotrophic lateral sclerosis (ALS), schizophrenia, a brain injury, a stroke, an ischemia, multiple sclerosis, epilepsy, spongiform encephalopathies, frontotemporal lobular degeneration, pugilistic encephalitis, AIDS associated dementia, COVID associated neurodegeneration, a mood disorder, depression, and bipolar disorder.

127. The method of any one of claims 1-126, wherein the disease or condition is selected from the group consisting of a vascular dementia, a tauopathies, a progressive supranuclear palsy, a corticobasal degeneration, a subacute sclerosing panencephalitic parkinsonism, a postencephalitic parkinsonism, a guam parkinsonism-dementia complex, a Pick's disease, and a frontotemporal dementia.

128. The method of any one of claims 1-127, wherein the compound is administered systemically.

129. The method of any one of claims 1-128, wherein the compound is administered orally.

130. The method of any one of claims 1-128, wherein the compound is administered intravenously.

131. The method of any one of claims 1-130, wherein the subject is a mammal.

132. The method of claim 131, wherein the subject is a human.
